(19) United States
(12) Patent Application Publication
(10) Pub. No.: US 2019/0292215 A1
(43) Pub. Date: Sep. 26, 2019

(54) COMPOUNDS, COMPOSITIONS, AND METHODS FOR THE TREATMENT OF DISEASE

(71) Applicant: Sperovie Biosciences, Inc., Hopkinton, MA (US)

(72) Inventors: Radhakrishnan P. Iyer, Shrewsbury, MA (US); Seetharamaiyer Padmanabhan, Lexington, MA (US); Dillon Cleary, Middleborough, MA (US); Geeta Meher, Milford, MA (US); Rayomand H. Gimi, Chelmsford, MA (US); Anjaneyulu Sheri, Shrewsbury, MA (US); Shenghua Zhou, Shrewsbury, MA (US); Sreerupa Challa, Shrewsbury, MA (US)

(21) Appl. No.: 16/317,746

(22) PCT Filed: Jul. 14, 2017
(86) PCT No.: PCT/US17/42106

§ 371 Date: Jan. 14, 2019

Related U.S. Application Data
(60) Provisional application No. 62/411,405, filed on Oct. 21, 2016.
(60) Provisional application No. 62/363,123, filed on Jul. 15, 2016.

(51) Int. Cl. C07H 21/00; A61K 31/7084; A61K 45/06; A61P 37/02

(52) U.S. Cl. CPC C07H 21/00; A61P 37/02; A61K 45/06; A61K 31/7084

(57) ABSTRACT

Disclosed are compounds and compositions for the induction of expression of a pattern recognition receptor (e.g., STING) and methods of use thereof.

### RELATED APPLICATIONS

This application claims the benefit of priority to U.S. Provisional Patent Application Ser. No. 62/363,123, filed Jul. 15, 2016; and Ser. No. 62/411,405, filed Oct. 21, 2016.

### FIELD OF INVENTION

This invention relates to compounds and compositions that activate the innate immune defense system and induce expression of pattern recognition receptors in a host, as well as methods of use for the treatment of a proliferative disease (e.g., cancer).

### BACKGROUND OF INVENTION

A key feature of the innate immune system is the recognition and elimination of foreign substances. Identification of these pathogenic invaders occurs through host recognition of evolutionarily conserved microbial structures known as pathogen-associated molecular patterns (PAMPs) (Jensen, S. and Thomsen, A. R. J Virol (2012) 86:2900-2910). These PAMPs include a wide array of molecular structures, such as nucleic acids, lipopolysaccharides, and glycoproteins that may be broadly shared by multiple microbial species and are critical to their survival and/or pathogenicity. Host recognition may occur by multiple pathways, such as activation of pattern recognition receptors (PRRs), which ultimately lead to downstream signaling events and culminate in the mounting of an immune response.

To date, several PRRs have been identified that serve as sensors of pathogenic infection. For example, the retinoic acid-inducible gene-I (RIG-I) protein is a RNA helicase that also functions as a sensor of microbial-derived RNA. RIG-I is important factor in host recognition of RNA viruses from a variety of different viral families, including Flaviviridae (e.g., West Nile virus, Hepatitis C virus, Japanese encephalitis virus, Dengue virus), Paramyxoviridae (e.g., Sendai virus, Newcastle disease virus, Respiratory syncytial virus, Measles virus), Rhabdoviridae (e.g., Rabies virus), Orthomyxoviridae (e.g., influenza A virus, influenza B virus), and Arenaviridae (e.g., Lassa virus), as well as a biomarker for the prediction of prognosis for certain types of cancer, such as hepatocellular carcinoma (Hou, J. et al, Cancer Cell (2014) 25:49-63). The stimulator of interferon genes (STING) is a cytoplasmic adaptor protein that activates the TBK1-IRF3 signaling complex, resulting in induction of type I interferons (IFN-β and IFN-α) and other immune pathway proteins. Other PRRs also play a role in sensing microbial-derived nucleic acids, including NOD2, LGP2, MDA5, and a number of Toll-like receptors (TLRs) that are expressed on the cell surface and within endosomal compartments.

Recent publications have highlighted the importance of RIG-I and STING as mediators of innate and adaptive immunity, and RIG-I and STING agonists have been recognized as immuno-oncology agents in cancer therapy (Li, X. Y. et al, Mol Cell Oncol (2014) 1:e968016; Woo, S. R. Trends in Immunol (2015) 36:250-256). In particular, RIG-I is involved in the regulation of basic cellular processes such as hematopoietic proliferation and differentiation, maintenance of leukemic stemness, and tumorigenesis of hepatocellular carcinoma, indicating that RIG-I performs an essential function as a tumor suppressor. Importantly, the STING pathway of cytosolic DNA sensing has been shown to play an important mechanistic role in innate immune sensing, driving type I IFN production in cancer and in the context of immune-oncology applications including therapeutics and diagnostics.

### SUMMARY OF INVENTION

Acyclic dinucleotide compounds, compositions comprising acyclic dinucleotide compounds, compositions, and related methods of use are described herein.

In one aspect, the invention features a compound of Formula (I):

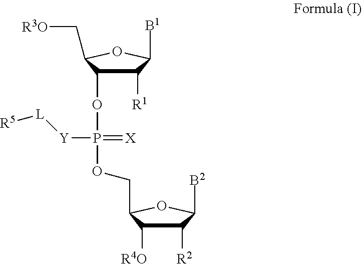

or a pharmaceutically acceptable salt, wherein:

each of B¹ and B² is independently a purinyl nucleobase or pyrimidinyl nucleobase;

X is O or S;

Y is O, S, or NR⁶;

L is absent, C₁-C₆ alkyl or C₁-C₆ heteroalkyl, wherein each C₁-C₆ alkyl and C₁-C₆ heteroalkyl is optionally substituted with R⁷;

each of R¹ and R² is independently hydrogen, halo, —CN, C₁-C₂₀ alkyl (e.g., C₁-C₆ alkyl), or OR⁸, provided that at least one of R¹ and R² is halo, O—C₁-C₂₀-alkenyl, or O—C₁-C₂₀-alkynyl or R¹ is hydrogen;

each of R³ and R⁴ is independently hydrogen or C₁-C₂₀ alkyl (e.g., C₁-C₆ alkyl).

R⁵ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹;

R⁶ is hydrogen or C₁-C₂₀ alkyl (e.g., C₁-C₆ alkyl);

R⁷ is halo, —CN, C₁-C₂₀ alkyl (e.g., C₁-C₆ alkyl), OR⁸, oxo, cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

R⁸ is hydrogen, C₁-C₂₀ alkynyl (e.g., C₁-C₆ alkynyl), C₁-C₂₀ alkenyl (e.g., C₁-C₆ alkenyl), cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

each R⁹ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R¹⁰; and

each R¹⁰ is independently C₁-C₂₀ alkyl, halo, —CN, OH, O—C₁-C₂₀ alkyl, O—C₁-C₂₀ heteroalkyl, O-aryl, or O-heteroaryl.

In some embodiments, at least one of B¹ or B² is a purinyl nucleobase. In some embodiments, each of B¹ or B² is independently a purinyl nucleobase. In some embodiments, B¹ is a purinyl nucleobase. In some embodiments, B² is a pyrimidinyl nucleobase. In some embodiments, B¹ is a purinyl nucleobase and B² is a pyrimidinyl nucleobase. In some embodiments, B¹ is adenosinyl or guanosinyl. In some embodiments, B² is cytosinyl, thyminyl, or uracilyl. In some embodiments, B¹ is adenosinyl or guanosinyl and B² is cytosinyl, thyminyl, or uracilyl. In some embodiments, each of B¹ is and B² is independently uracilyl. In some embodiments, each of B¹ is and B² is independently adenosinyl.

In some embodiments, each of R¹ and R² is independently hydrogen, fluorine, C₁-C₂₀ alkyl (e.g., C₁-C₆ alkyl), C₁-C₂₀ alkenyl (e.g., C₁-C₆ alkenyl), or O—C₁-C₂₀ alkynyl (e.g., C₁-C₆ alkynyl).

In some embodiments, each of R¹ and R² is independently fluorine.

In some embodiments, the compound is a compound of Formula (II):

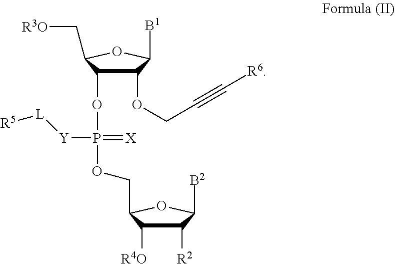

In some embodiments, R⁶ is hydrogen, C₁-C₂₀ alkyl (e.g., C₁-C₆ substituted or unsubstituted alkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹.

In some embodiments, the compound is selected from the following:

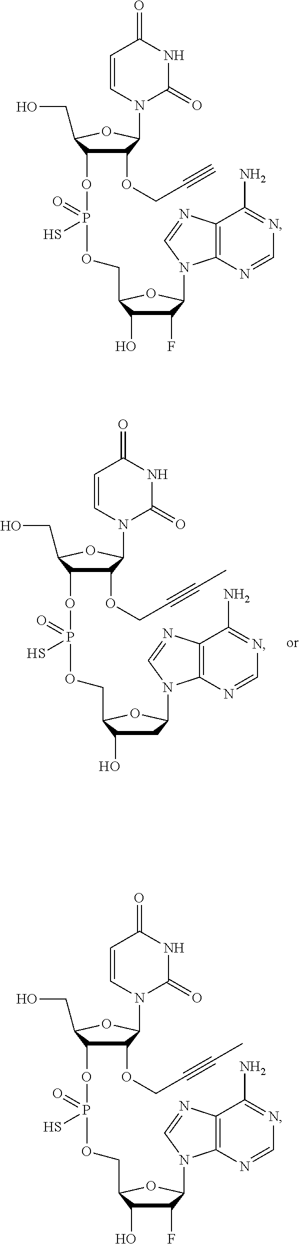

or a pharmaceutically acceptable salt thereof.

In some embodiments, the compound is selected from:

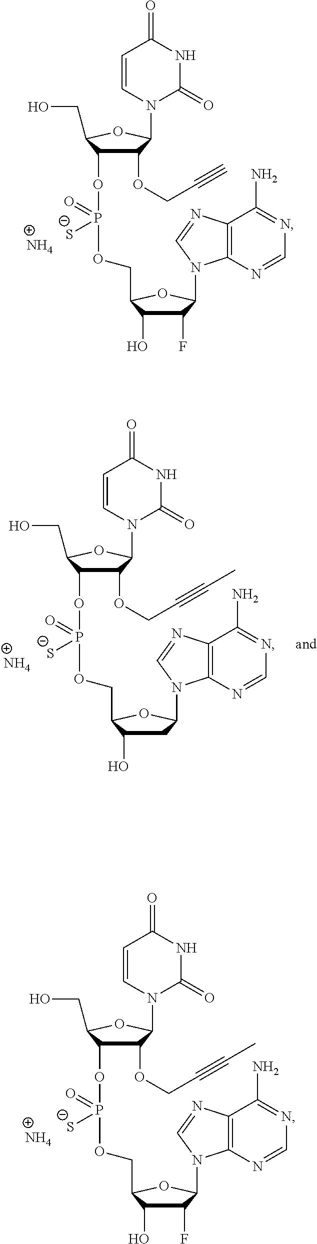

or a pharmaceutically acceptable salt thereof.

In some embodiments, the compound is selected from:

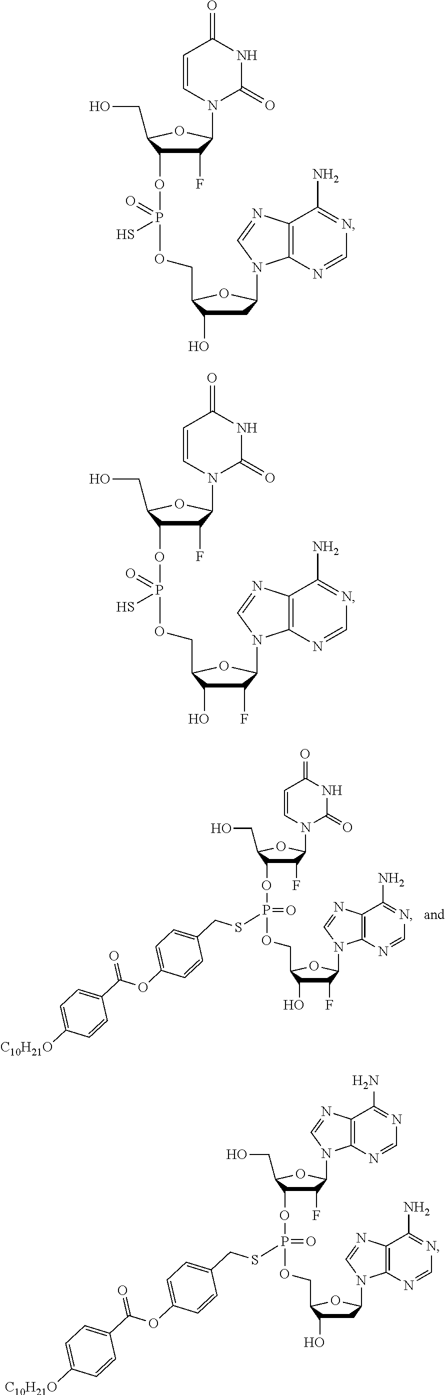

or a pharmaceutically acceptable salt thereof.

In some embodiments, the compound is selected from:

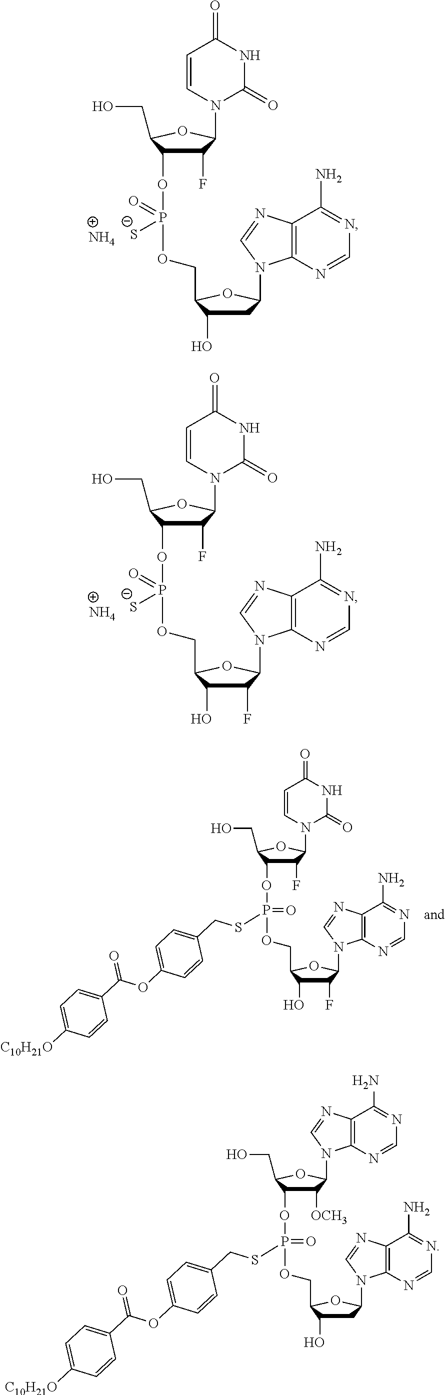

In some embodiments, the compound is selected from:

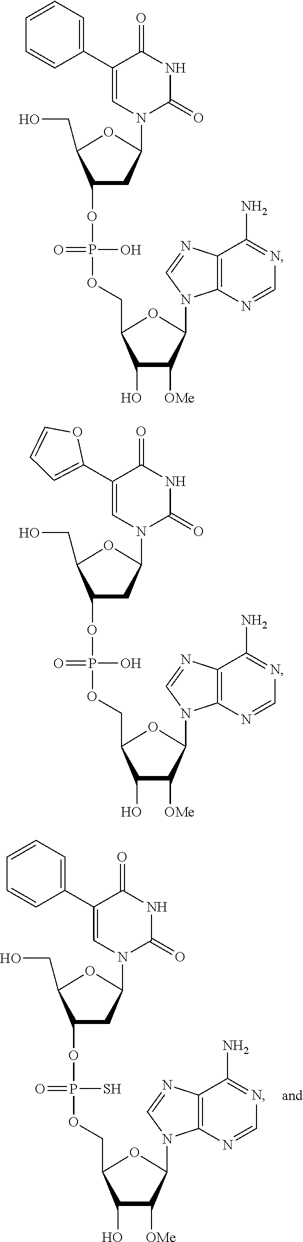

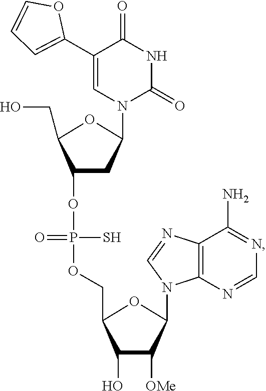

or a pharmaceutically acceptable salt thereof.

In some embodiments, the compound is selected from:

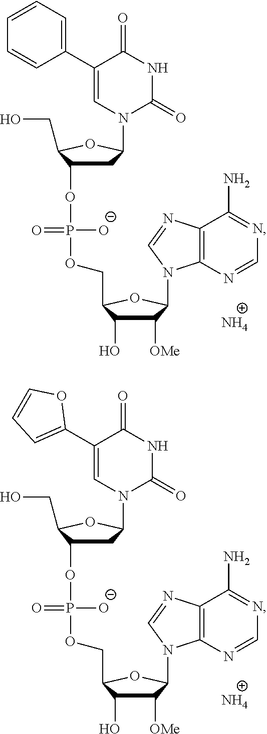

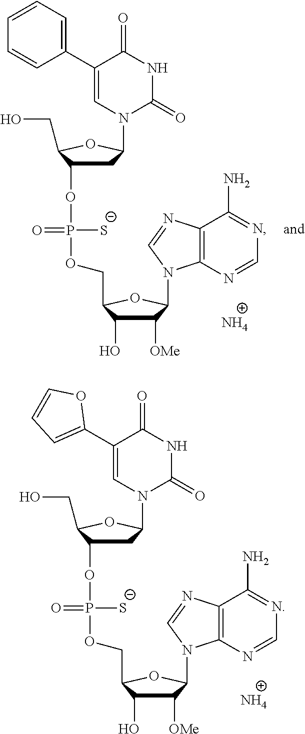

In another aspect, the invention describes a compound of Formula (III-a) or (III-b):

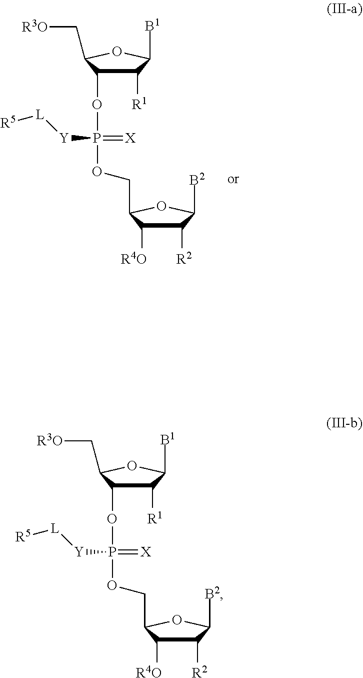

or pharmaceutically acceptable salts thereof, wherein the composition is a mixture of a compound of Formula (III-a) or (III-b).

In some embodiments, the composition is an optically enriched mixture of a compound of Formula (III-a) or (III-b).

In some embodiments, the composition comprises a compound of Formula (III-a) or (III-b) in an enantiomeric excess of 90%.

In another aspect, the invention features a compound of Formula (IV):

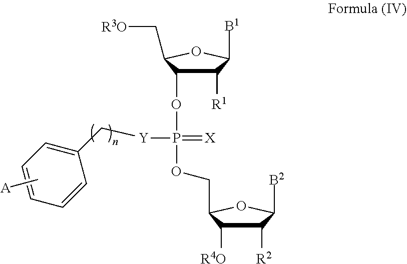

or a pharmaceutically acceptable salt or stereoisomer thereof, wherein:

each of B¹ and B² is independently a purinyl nucleobase or pyrimidinyl nucleobase;

X is O or S;

Y is O, S, or NR⁵;

n is 1, 2, or 3;

each of R¹ and R² is independently hydrogen, —CN, C₁-C₂₀ alkyl (e.g., C₁-C₆ alkyl), or OR⁶;

each of R³ and R⁴ is independently hydrogen or C₁-C₂₀ alkyl (e.g., C₁-C₆ alkyl);

R⁵ is hydrogen or C₁-C₂₀ alkyl (e.g., C₁-C₆ alkyl);

R⁶ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁷;

each R⁷ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R⁸;

each R⁸ is independently C₁-C₂₀ alkyl, halo, —CN, OH, O—C₁-C₂₀ alkyl, O—C₁-C₂₀ heteroalkyl, O-aryl, or O-heteroaryl; and

A is OC(O)—C₆-C₂₀ alkyl or OC(O)-aryl, wherein aryl is optionally substituted with C₆-C₂₀ alkyl, O—C₆-C₂₀ alkyl or C₁-C₆—O—C₆-C₂₀ alkyl.

In some embodiments, each of R¹ and R² is independently hydrogen or O—C₁-C₂₀ alkyl.

In some embodiments, A is OC(O)—C₆-C₂₀ alkyl or OC(O)-aryl, wherein aryl is substituted with C₆-C₂₀ alkyl, O—C₆-C₂₀ alkyl or C₁-C₆—O—C₆-C₂₀ alkyl.

In some embodiments, each of R³ and R⁴ is independently hydrogen.

In some embodiments, R¹ is O—C₁-C₂₀ alkyl and R² is hydrogen.

In some embodiments, the compound of Formula (IV) is selected from:

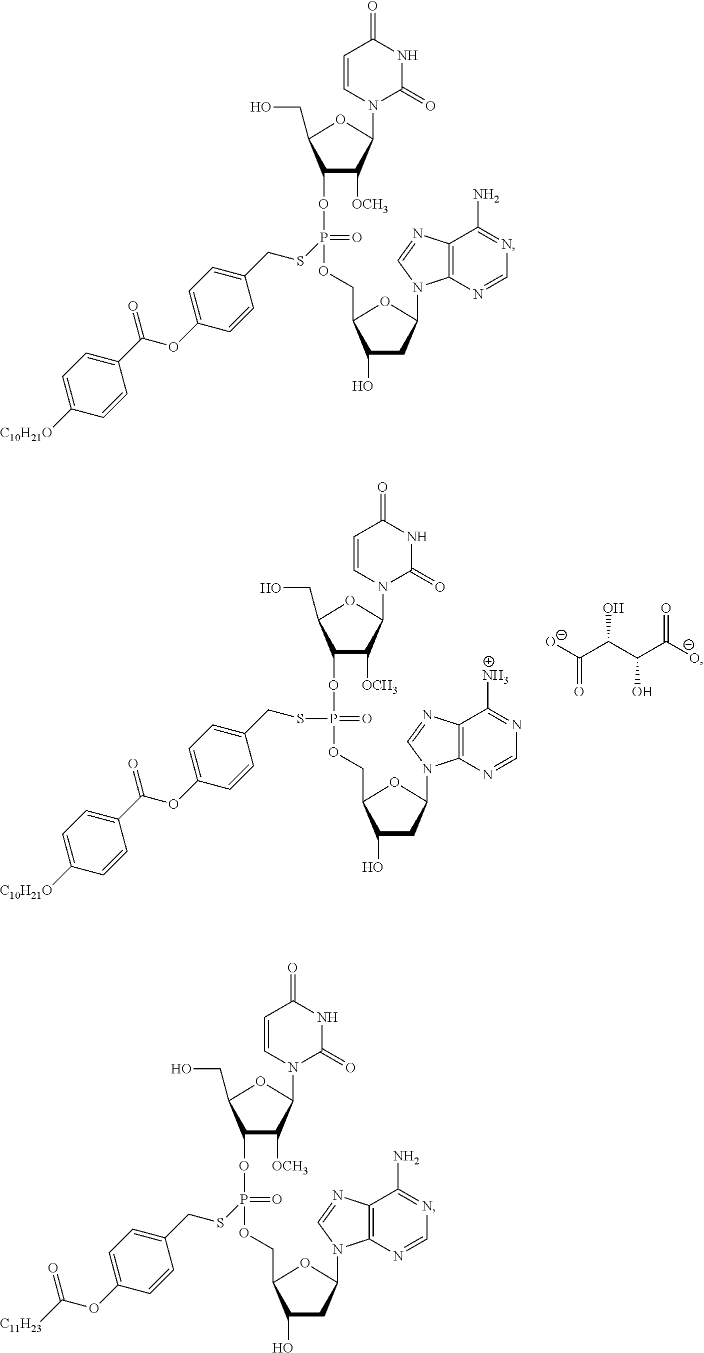

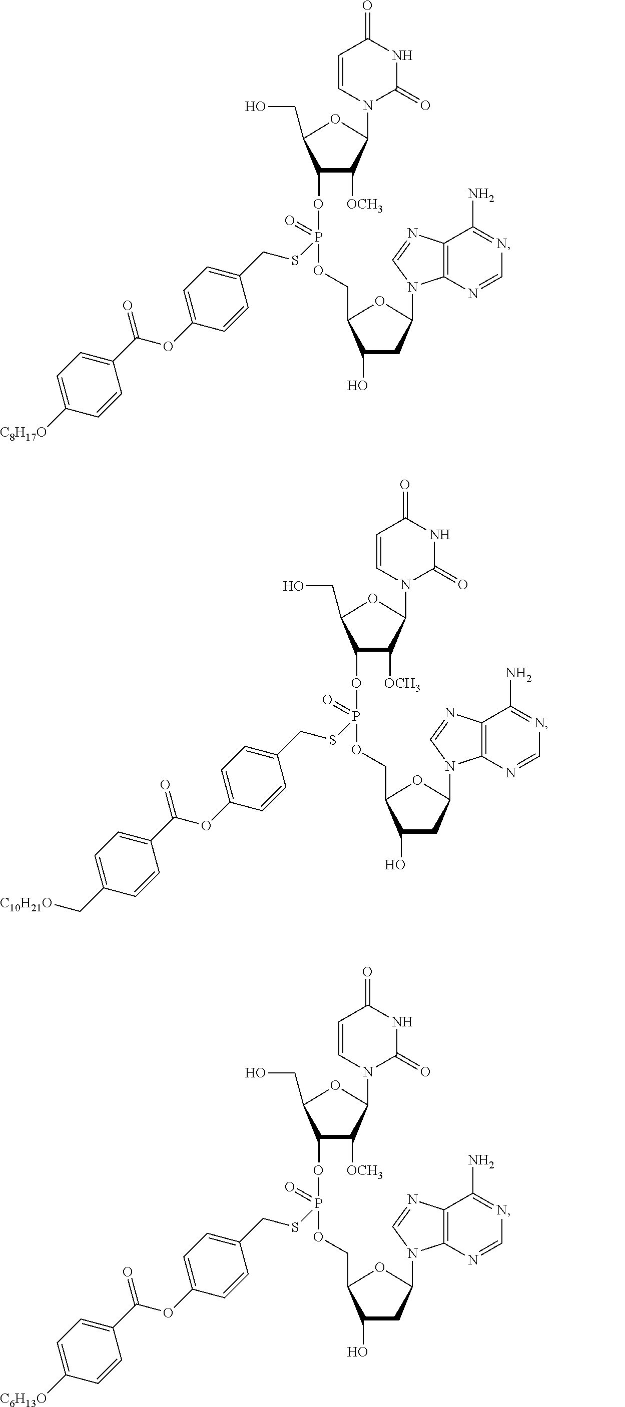

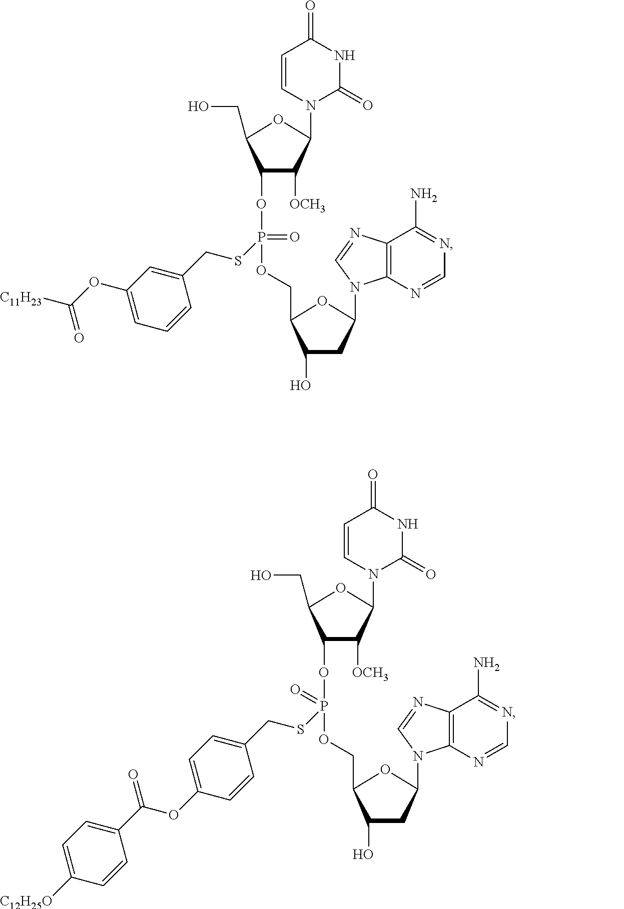

or a pharmaceutically acceptable salt thereof.

In another aspect, the present invention features a composition comprising a compound of Formula (V-a) or (V-b):

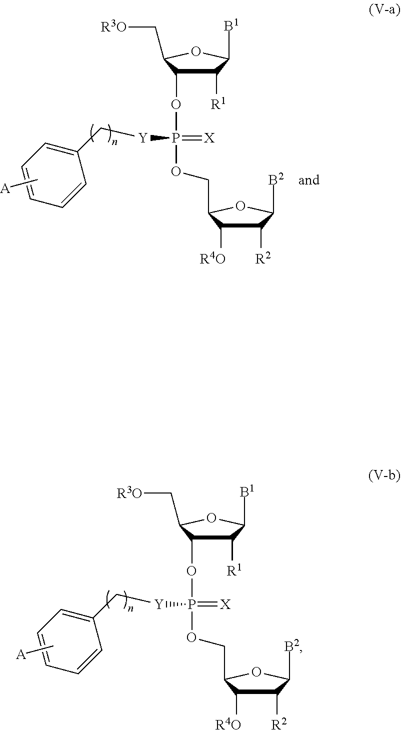

or a pharmaceutically acceptable salt thereof, wherein the composition is an optically enriched mixture of Formula (V-a) or (V-b).

In some embodiments, the composition is an optically enriched mixture of a compound of Formula (V-a) or (V-b).

In some embodiments, the composition comprises a compound of Formula (V-a) or (V-b) in an enantiomeric excess of 90%.

In some embodiments, the composition comprises a compound selected from:

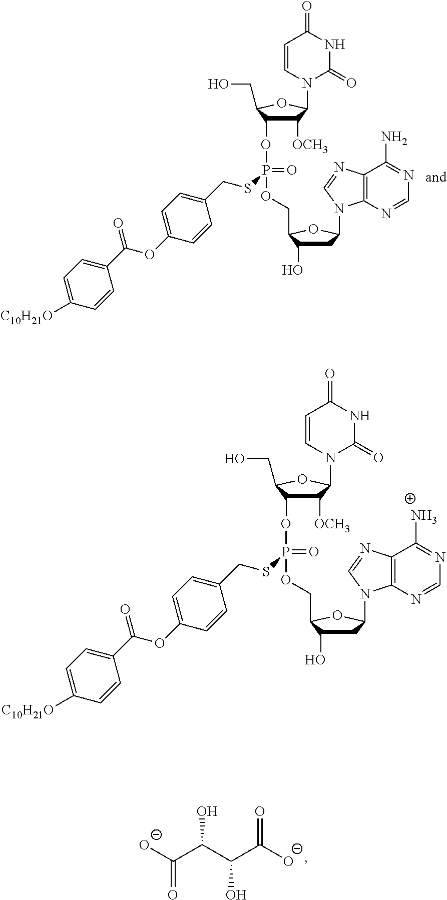

or a pharmaceutically acceptable salt thereof.

In another aspect, the invention describes herein a method of treating cancer in a subject, the method comprising administering to the subject an effective amount of a compound of Formula (I),

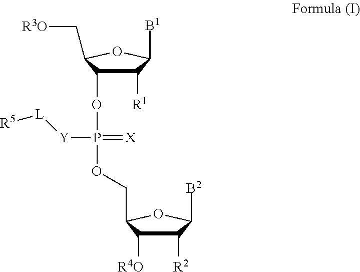

or a pharmaceutically acceptable salt, wherein:

each of B¹ and B² is independently a purinyl nucleobase or pyrimidinyl nucleobase;

X is O or S;

Y is O, S, or NR⁶;

L is absent, C₁-C₆ alkyl or C₁-C₆ heteroalkyl, wherein each C₁-C₆ alkyl and C₁-C₆ heteroalkyl is optionally substituted with R⁷;

each of R¹ and R² is independently hydrogen, halo, —CN, C₁-C₂₀ alkyl (e.g., C₁-C₆ alkyl), or OR⁸, provided that at least one of R¹ and R² is halo, O—C₁-C₂₀-alkenyl, or O—C₁-C₂₀-alkynyl or R¹ is hydrogen;

each of R³ and R⁴ is independently hydrogen or C₁-C₂₀ alkyl (e.g., C₁-C₆ alkyl).

R⁵ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹;

R⁶ is hydrogen or C₁-C₂₀ alkyl (e.g., C₁-C₆ alkyl);

R⁷ is halo, —CN, C₁-C₂₀ alkyl (e.g., C₁-C₆ alkyl), OR⁸, oxo, cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

R⁸ is hydrogen, C₁-C₂₀ alkynyl (e.g., C₁-C₆ alkynyl), C₁-C₂₀ alkenyl (e.g., C₁-C₆ alkenyl), cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

each R⁹ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R¹⁰; and

each R¹⁰ is independently C₁-C₂₀ alkyl, halo, —CN, OH, O—C₁-C₂₀ alkyl, O—C₁-C₂₀ heteroalkyl, O-aryl, or O-heteroaryl.

In some embodiments, the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

In some embodiments, the cancer is a cancer of the liver.

In some embodiments, any of the above methods within this aspect further comprise administration of an additional agent (e.g., an anticancer agent).

In some embodiments, the additional agent comprises methotrexate, 5-fluorouracil, doxorubicin, vincristine, bleomycin, vinblastine, dacarbazine, toposide, cisplatin, epirubicin, or sorafenib tosylate.

In another aspect, the invention describes herein a method of inducing the expression of a pattern recognition receptors (PRRs) for immune-modulation in a subject, the method comprising administering to the subject an effective amount of a compound of Formula (I),

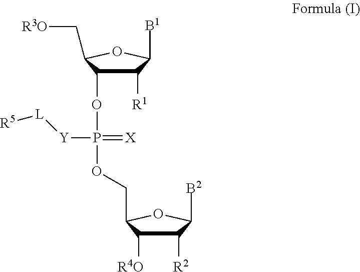

or a pharmaceutically acceptable salt, wherein:

each of B¹ and B² is independently a purinyl nucleobase or pyrimidinyl nucleobase;

X is O or S;

Y is O, S, or NR⁶;

L is absent, C₁-C₆ alkyl or C₁-C₆ heteroalkyl, wherein each C₁-C₆ alkyl and C₁-C₆ heteroalkyl is optionally substituted with R⁷;

each of R¹ and R² is independently hydrogen, halo, —CN, C₁-C₂₀ alkyl (e.g., C₁-C₆ alkyl), or OR⁸, provided that at least one of R¹ and R² is halo, O—C₁-C₂₀-alkenyl, or O—C₁-C₂₀-alkynyl or R¹ is hydrogen;

each of R³ and R⁴ is independently hydrogen or C₁-C₂₀ alkyl (e.g., C₁-C₆ alkyl).

R⁵ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹;

R⁶ is hydrogen or C₁-C₂₀ alkyl (e.g., C₁-C₆ alkyl);

R⁷ is halo, —CN, C₁-C₂₀ alkyl (e.g., C₁-C₆ alkyl), OR⁸, oxo, cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

R⁸ is hydrogen, C₁-C₂₀ alkynyl (e.g., C₁-C₆ alkynyl), C₁-C₂₀ alkenyl (e.g., C₁-C₆ alkenyl), cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

each R⁹ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R¹⁰; and

each R¹⁰ is independently C₁-C₂₀ alkyl, halo, —CN, OH, O—C₁-C₂₀ alkyl, O—C₁-C₂₀ heteroalkyl, O-aryl, or O-heteroaryl.

In another aspect, the invention describes herein a method of inducing the expression of a pattern recognition receptors for immunomodulation and inducing a therapeutic response in a subject having cancer, the method comprising administering to the subject an effective amount of a compound of Formula (I),

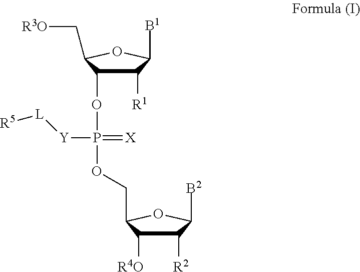

or a pharmaceutically acceptable salt, wherein:

each of B¹ and B² is independently a purinyl nucleobase or pyrimidinyl nucleobase;

X is O or S;

Y is O, S, or NR⁶;

L is absent, C₁-C₆ alkyl or C₁-C₆ heteroalkyl, wherein each C₁-C₆ alkyl and C₁-C₆ heteroalkyl is optionally substituted with R⁷;

each of R¹ and R² is independently hydrogen, halo, —CN, C₁-C₂₀ alkyl (e.g., C₁-C₆ alkyl), or OR⁸, provided that at least one of R¹ and R² is halo, O—C₁-C₂₀-alkenyl, or O—C₁-C₂₀-alkynyl or R¹ is hydrogen;

each of R³ and R⁴ is independently hydrogen or C₁-C₂₀ alkyl (e.g., C₁-C₆ alkyl).

R⁵ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹;

R⁶ is hydrogen or C₁-C₂₀ alkyl (e.g., C₁-C₆ alkyl);

R⁷ is halo, —CN, C₁-C₂₀ alkyl (e.g., C₁-C₆ alkyl), OR⁸, oxo, cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

R⁸ is hydrogen, C₁-C₂₀ alkynyl (e.g., C₁-C₆ alkynyl), C₁-C₂₀ alkenyl (e.g., C₁-C₆ alkenyl), cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

each R⁹ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R¹⁰; and

each R¹⁰ is independently C₁-C₂₀ alkyl, halo, —CN, OH, O—C₁-C₂₀ alkyl, O—C₁-C₂₀ heteroalkyl, O-aryl, or O-heteroaryl.

In another aspect, the invention describes herein a method of treating cancer in a subject, the method comprising administering to the subject an effective amount of a composition comprising compounds of Formula (III-a) or (III-b),

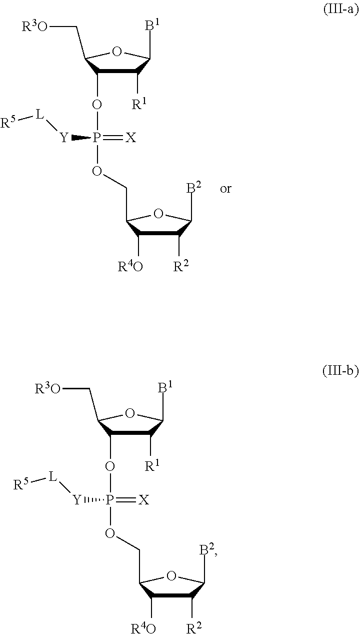

or pharmaceutically acceptable salts thereof, wherein the composition is a mixture of a compound of Formula (III-a) or (III-b).

In some embodiments, the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

In some embodiments, the cancer is a cancer of the liver.

In some embodiments, any of the above methods within this aspect further comprises administration of an additional agent (e.g., an anticancer agent).

In some embodiments, the additional agent comprises methotrexate, 5-fluorouracil, doxorubicin, vincristine, bleomycin, vinblastine, dacarbazine, toposide, cisplatin, epirubicin, or sorafenib tosylate.

In another aspect, the invention describes herein a method of inducing the expression of a pattern recognition receptors (PRRs) for immune-modulation in a subject, the method comprising administering to the subject an effective amount of a composition comprising compounds of Formula (III-a) or (III-b),

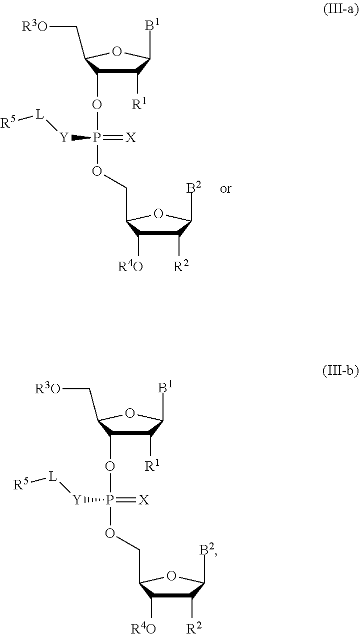

or pharmaceutically acceptable salts thereof, wherein the composition is a mixture of a compound of Formula (III-a) or (III-b).

In another aspect, the invention describes herein a method of inducing the expression of a pattern recognition receptors for immunomodulation and inducing a therapeutic response in a subject having cancer, the method comprising administering to the subject an effective amount of a composition comprising compounds of Formula (III-a) or (III-b),

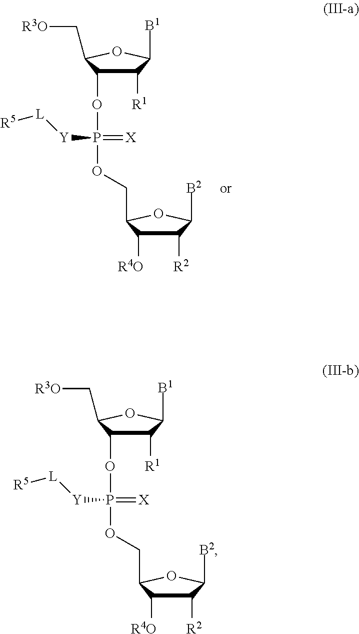

or pharmaceutically acceptable salts thereof, wherein the composition is a mixture of a compound of Formula (III-a) or (III-b).

In another aspect, the invention describes herein a method of treating cancer in a subject, the method comprising administering to the subject an effective amount of a compound of Formula (IV),

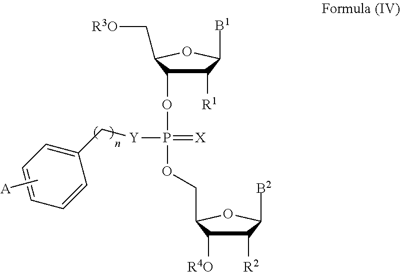

or a pharmaceutically acceptable salt or stereoisomer thereof, wherein:

each of B¹ and B² is independently a purinyl nucleobase or pyrimidinyl nucleobase;

X is O or S;

Y is O, S, or NR⁵;

n is 1, 2, or 3;

each of R¹ and R² is independently hydrogen, —CN, C₁-C₂₀ alkyl (e.g., C₁-C₆ alkyl), or OR⁶;

each of R³ and R⁴ is independently hydrogen or C₁-C₂₀ alkyl (e.g., C₁-C₆ alkyl);

R⁵ is hydrogen or C₁-C₂₀ alkyl (e.g., C₁-C₆ alkyl);

R⁶ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁷;

each R⁷ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R⁸;

each R⁸ is independently C₁-C₂₀ alkyl, halo, —CN, OH, O—C₁-C₂₀ alkyl, O—C₁-C₂₀ heteroalkyl, O-aryl, or O-heteroaryl; and

A is OC(O)—C₆-C₂₀ alkyl or OC(O)-aryl, wherein aryl is optionally substituted with C₆-C₂₀ alkyl, O—C₆-C₂₀ alkyl or C₁-C₆—O—C₆-C₂₀ alkyl.

In some embodiments, the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

In some embodiments, the cancer is a cancer of the liver.

In some embodiments, any of the above methods within this aspect further comprises administration of an additional agent (e.g., an anticancer agent).

In some embodiments, the additional agent comprises methotrexate, 5-fluorouracil, doxorubicin, vincristine, bleomycin, vinblastine, dacarbazine, toposide, cisplatin, epirubicin, or sorafenib tosylate.

In another aspect, the invention describes herein a method of inducing the expression of a pattern recognition receptors (PRRs) for immune-modulation in a subject, the method comprising administering to the subject an effective amount of a compound of Formula (IV),

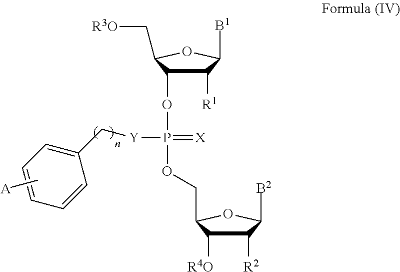

or a pharmaceutically acceptable salt or stereoisomer thereof, wherein:

each of B¹ and B² is independently a purinyl nucleobase or pyrimidinyl nucleobase;

X is O or S;

Y is O, S, or NR⁵;

n is 1, 2, or 3;

each of R¹ and R² is independently hydrogen, —CN, C₁-C₂₀ alkyl (e.g., C₁-C₆ alkyl), or OR⁶;

each of R³ and R⁴ is independently hydrogen or C₁-C₂₀ alkyl (e.g., C₁-C₆ alkyl);

R⁵ is hydrogen or C₁-C₂₀ alkyl (e.g., C₁-C₆ alkyl);

R⁶ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁷;

each R⁷ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R⁸;

each R⁸ is independently C₁-C₂₀ alkyl, halo, —CN, OH, O—C₁-C₂₀ alkyl, O—C₁-C₂₀ heteroalkyl, O-aryl, or O-heteroaryl; and

A is OC(O)—C₆-C₂₀ alkyl or OC(O)-aryl, wherein aryl is optionally substituted with C₆-C₂₀ alkyl, O—C₆-C₂₀ alkyl or C₁-C₆—O—C₆-C₂₀ alkyl.

In another aspect, the invention describes herein a method of inducing the expression of a pattern recognition receptors for immunomodulation and inducing a therapeutic response in a subject having cancer, the method comprising administering to the subject an effective amount of a compound of Formula (IV),

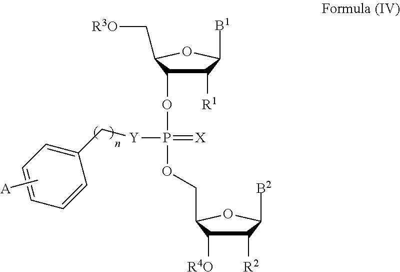

or a pharmaceutically acceptable salt or stereoisomer thereof, wherein:

each of B¹ and B² is independently a purinyl nucleobase or pyrimidinyl nucleobase;

X is O or S;

Y is O, S, or NR;

n is 1, 2, or 3;

each of R¹ and R² is independently hydrogen, —CN, C₁-C₂₀ alkyl (e.g., C₁-C₆ alkyl), or OR⁶;

each of R³ and R⁴ is independently hydrogen or C₁-C₂₀ alkyl (e.g., C₁-C₆ alkyl);

R⁵ is hydrogen or C₁-C₂₀ alkyl (e.g., C₁-C₆ alkyl);

R⁶ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁷;

each R⁷ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R⁸;

each R⁸ is independently C₁-C₂₀ alkyl, halo, —CN, OH, O—C₁-C₂₀ alkyl, O—C₁-C₂₀ heteroalkyl, O-aryl, or O-heteroaryl; and

A is OC(O)—C₆-C₂₀ alkyl or OC(O)-aryl, wherein aryl is optionally substituted with C₆-C₂₀ alkyl, O—C₆-C₂₀ alkyl or C₁-C₆—O—C₆-C₂₀ alkyl.

In another aspect, the invention describes herein a method of treating cancer in a subject, the method comprising administering to the subject an effective amount of a composition comprising compounds of Formula (V-a) or (V-b),

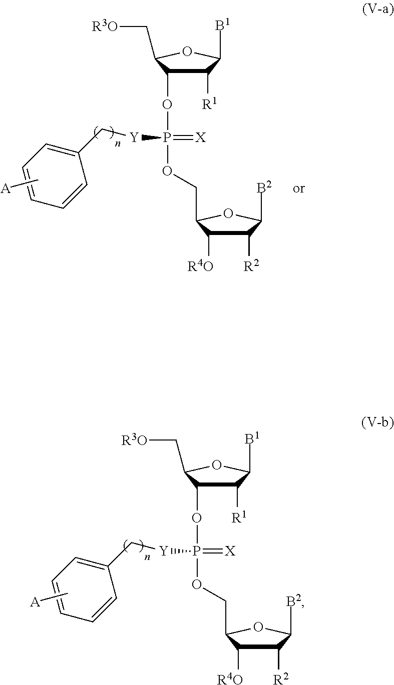

or pharmaceutically acceptable salts thereof, wherein the composition is a mixture of a compound of Formula (V-a) or (V-b).

In some embodiments, the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

In some embodiments, the cancer is a cancer of the liver.

In some embodiments, any of the above methods within this aspect further comprises administration of an additional agent (e.g., an anticancer agent).

In some embodiments, the additional agent comprises methotrexate, 5-fluorouracil, doxorubicin, vincristine, bleomycin, vinblastine, dacarbazine, toposide, cisplatin, epirubicin, or sorafenib tosylate.

In another aspect, the invention describes herein a method of inducing the expression of a pattern recognition receptors (PRRs) for immune-modulation in a subject, the method comprising administering to the subject an effective amount of a composition comprising compounds of Formula (V-a) or (V-b),

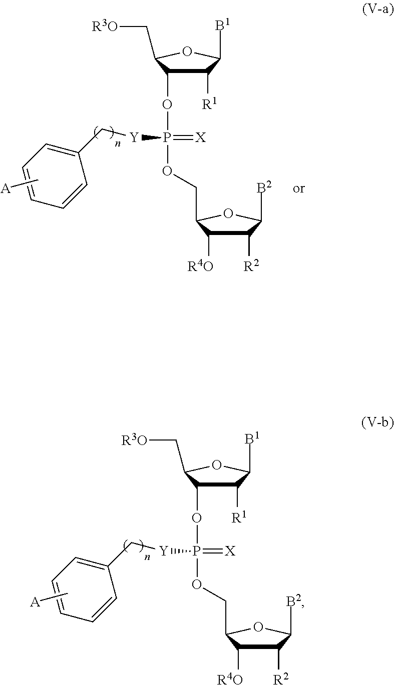

or pharmaceutically acceptable salts thereof, wherein the composition is a mixture of a compound of Formula (V-a) or (V-b).

In another aspect, the invention describes herein a method of inducing the expression of a pattern recognition receptors for immunomodulation and inducing a therapeutic response in a subject having cancer, the method comprising administering to the subject an effective amount of a composition comprising compounds of Formula (V-a) or (V-b),

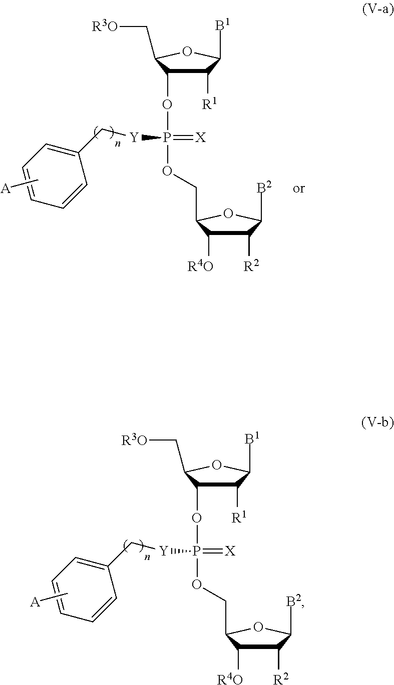

or pharmaceutically acceptable salts thereof, wherein the composition is a mixture of a compound of Formula (V-a) or (V-b).

### BRIEF DESCRIPTION OF THE DRAWINGS

FIG. 1 shows a table of exemplary compounds of the invention.

FIG. 2 shows exemplary compounds that activate ISG54-specific SEAP production in THP1-Blue ISG cells.

FIG. 3 shows IRF induction by an exemplary compound in THP1 Cells.

FIG. 4 shows an exemplary compound that induces a STING-dependent type I IFN response in THP1 cells in a dose-dependent manner.

FIG. 5A shows IRF activity of exemplary compounds.

FIG. 5B shows a cytotoxicity assay of exemplary compounds.

FIG. 6 shows IRF induction by exemplary compounds is STING-dependent.

FIG. 7 shows STING pathway plays a critical role in type I IFN response induced by compounds in THP1 cells.

FIG. 8 shows IRF induction by an exemplary compound in THP1 Cells.

FIG. 9 shows exemplary compounds induce dose-dependent ISG54-specific SEAP production in THP1-Blue ISG cells.

FIG. 10 shows IRF-, and NF-kB-inducing activity of exemplary compounds.

FIG. 11 shows IRF induction by exemplary compounds is STING-dependent.

FIG. 12 shows IRF induction by an exemplary compound in THP1 cells.

FIG. 13 shows IRF induction by an exemplary compound in THP1 cells.

FIG. 14 shows IRF induction by an exemplary compound in THP1 cells.

FIG. 15 shows IRF Induction by exemplary compounds.

FIG. 16 shows an exemplary compound induces a STING-dependent type I IFN response in THP1 cells.

FIG. 17 shows that an exemplary compound induces the expression of IFN-β and IRF7 in THP1 cells.

FIG. 18 shows 2′3′-cGAMP induces IFN-β gene expression within 5 hrs; it takes >5 hrs for an exemplary compound to activate IFN-β gene expression in THP1-WT.

FIG. 19 shows an exemplary compound that induces the expression of IFN-β and IRF7 in THP1 cells in STING-dependent manner.

FIG. 20 shows the cGAS pathway appears important for induced type I IFN responses from an exemplary compound.

FIG. 21 shows K384 and K411 residues in cGAS appear important in mediating an activation of STING-dependent type I IFN signaling with an exemplary compound.

FIG. 22 shows RIG-I, MDA5, LGP2, OAS1 and ISG54 gene expression in THP1 after a Poly IC & dsRNA treatment with an exemplary compound.

FIG. 23 shows dose dependent induction of various ISGs in THP1 cells by Cmd 7. Gene expression analysis in THP1 after treatment with an exemplary compound.

FIG. 24 is a chart showing that ATP and GTP enhance Cmd 1-induced type I IFN signaling in SZ14 cells. SZ14 were transfected with a cGAS expression plasmid for 24 hrs, followed by compound treatment in the presence of ATP and GTP (w/ATP & GTP) or absence of ATP and GTP (w/o ATP & GTP) for 21 hrs. ISG54 ISRE-luciferase activity was determined and shown as Relative Light Units (RLU) (average±standard deviation of triplicate wells). (Cmd 1 SB final concentration: 20 μM, ATP and GTP: 2 mM, 2′3′-cGAMP: 10 μM)

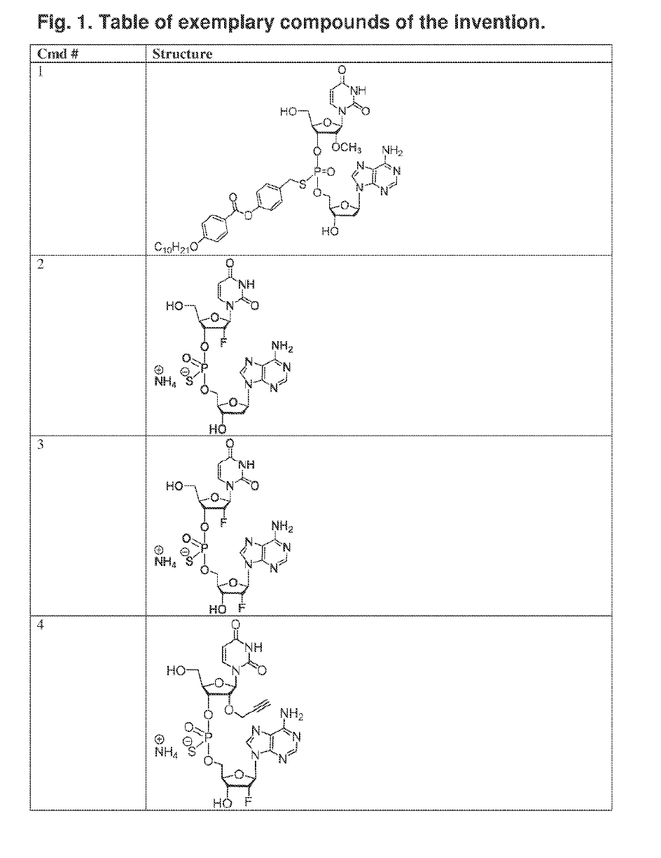

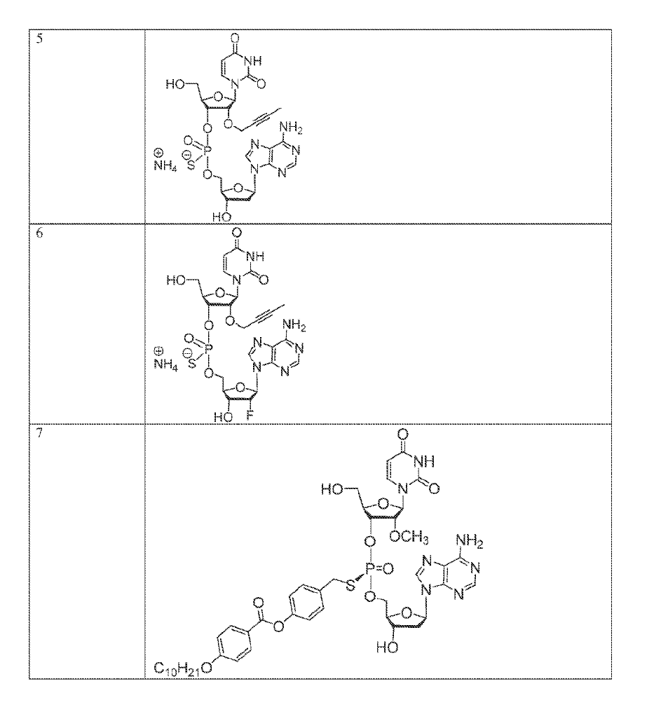

### DETAILED DESCRIPTION OF THE INVENTION

The present invention relates to methods of activating and/or inducing the expression of PRRs (e.g., STING) in a subject, in particular for the treatment of a proliferative disease (e.g., cancer). In some embodiments, the method comprises administration of a compound or composition described herein or pharmaceutically acceptable salt thereof. It is to be noted that induction of any PRR with these compounds can stimulate interferon and/or NF-KB production which can induce the expression of a variety of PRRs which are inducible genes by feedback mechanism.

### Definitions

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “acquire” or “acquiring” as the terms are used herein, refer to obtaining possession of a physical entity (e.g., a sample, e.g., blood sample or liver biopsy specimen), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of bodily fluid, such as blood by, e.g., mass spectroscopy, e.g. LC-MS.

As used herein, the terms “induce” or “induction of” refer to the increase or enhancement of a function, e.g., the increase or enhancement of the expression of a pattern recognition receptor (e.g, STING). In some embodiments, “induction of PRR expression” refers to induction of transcription of PRR RNA, e.g., STING RNA (e.g., mRNA, e.g., an increase or enhancement of), or the translation of a PRR protein, e.g., the STING protein (e.g., an increase or enhancement of). In some embodiments, induction of PRR expression (e.g., STING expression) refers to the increase or enhancement of the concentration of a PRR RNA, e.g., or STING RNA (e.g., mRNA) or the STING protein, e.g., in a cell. In some embodiments, induction of PRR expression (e.g., STING expression) refers to the increase of the number of copies of PRR RNA, e.g., STING RNA (e.g., mRNA) or PRR protein, e.g., the STING protein, e.g., in a cell. In some embodiments, to induce expression of a PRR (e.g., STING) may refer to the initiation of PRR RNA (e.g., STING RNA (e.g., mRNA)) or transcription or PRR protein (e.g., STING protein) translation. In some embodiments, to induce expression of a PRR (e.g., STING) may refer to an increase in the rate of PRR RNA (e.g., STING RNA (e.g., mRNA)) transcription or an increase in the rate of PRR protein (e.g., STING protein) expression.

As used herein, the terms “activate” or “activation” refer to the stimulation or triggering of a function, e.g., of a downstream pathway, e.g., a downstream signaling pathway. In some embodiments, activation of a pattern recognition receptor (PRR) (e.g., STING) refers to the stimulation of a specific protein or pathway, e.g., through interaction with a downstream signaling partner (e.g., IFN-β promoter stimulator 1 (IPS-1), IRF3, IRF7, NF-κB, interferons (e.g., IFN-α or IFN-β) and/or cytokines). In some embodiments, activation is distinct from the induction of expression of a PRR. In some embodiments, a PRR may be activated without resulting in an induction of PRR expression (e.g., expression of STING). In some embodiments, activation may include induction of expression of a PRR (e.g., STING). In some embodiments, activation of a PRR may trigger the induction of expression of a PRR (e.g., STING) by about 0.1%, about 0.5%, about 1%, about 5%, about 10%, about 15%, about 20%, about 25%, about 30%, about 40%, about 50%, about 60%, about 70%, about 80%, about 90%, about 95%, or more compared to a reference standard (e.g., basal expression levels of a PRR (e.g., STING)).

As used herein, an amount of a compound, conjugate, or substance effective to treat a disorder (e.g., a disorder described herein), “therapeutically effective amount,” “effective amount” or “effective course” refers to an amount of the compound, substance, or composition which is effective, upon single or multiple dose administration(s) to a subject, in treating a subject, or in curing, alleviating, relieving or improving a subject with a disorder (e.g., a microbial infection) beyond that expected in the absence of such treatment.

As used herein, the terms “prevent” or “preventing” as used in the context of a disorder or disease, refer to administration of an agent to a subject, e.g., the administration of a compound of the present invention to a subject, such that the onset of at least one symptom of the disorder or disease is delayed as compared to what would be seen in the absence of administration of said agent.

As used herein, the terms “reference treatment” or “reference standard” refer to a standardized level or standardized treatment that is used as basis for comparison. In some embodiments, the reference standard or reference treatment is an accepted, well known, or well characterized standard or treatment in the art. In some embodiments, the reference standard describes an outcome of a method described herein. In some embodiments, the reference standard describes a level of a marker (e.g., a level of induction of a PRR, e.g., STING) in a subject or a sample, e.g., prior to initiation of treatment, e.g., with a compound or composition described herein. In some embodiments, the reference standard describes a measure of the presence of, progression of, or severity of a disease or the symptoms thereof, e.g., prior to initiation of treatment, e.g., with a compound or composition described herein.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dogs, cats, cows, pigs, etc.

As used herein, the terms “treat” or “treating” a subject having a disorder or disease refer to subjecting the subject to a regimen, e.g., the administration of a compound or composition described herein or pharmaceutically acceptable salt thereof, or a composition comprising a compound or composition described herein or pharmaceutically acceptable salt thereof, such that at least one symptom of the disorder or disease is cured, healed, alleviated, relieved, altered, remedied, ameliorated, or improved. Treating includes administering an amount effective to alleviate, relieve, alter, remedy, ameliorate, improve or affect the disorder or disease, or the symptoms of the disorder or disease. The treatment may inhibit deterioration or worsening of a symptom of a disorder or disease.

As used herein, the term “Cmd” refers to the word “compound” or “Compound” to herein describe chemical compounds and used interchangeably.

As used herein, the term “Cmds” refers to the word “compounds” or “Compounds” to herein describe chemical compounds and used interchangeably.

Numerous ranges, e.g., ranges for the amount of a drug administered per day, are provided herein. In some embodiments, the range includes both endpoints. In other embodiments, the range excludes one or both endpoints. By way of example, the range can exclude the lower endpoint. Thus, in such an embodiment, a range of 250 to 400 mg/day, excluding the lower endpoint, would cover an amount greater than 250 that is less than or equal to 400 mg/day.

### Chemical Definitions

Certain compounds of the present invention can comprise one or more asymmetric centers, and thus can exist in various isomeric forms, e.g., stereoisomers and/or diastereomers. Thus, compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain embodiments, mixtures of stereoisomers or diastereomers are provided.

Where a particular enantiomer or diastereomer is preferred, it may, in some embodiments be provided substantially free of the corresponding enantiomer and/or diastereomers, and may also be referred to as “optically enriched.” “Optically-enriched,” as used herein, means that the compound is made up of a significantly greater proportion of one enantiomer or diastereomer. In certain embodiments the compound is made up of at least about 90% by weight of a preferred enantiomer or diastereomer. In other embodiments the compound is made up of at least about 95%, 98%, or 99% by weight of a preferred enantiomer or diastereomer. Preferred enantiomers or diastereomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972).

The compounds of this invention may contain one or more asymmetric centers and thus occur as racemates and racemic mixtures, single enantiomers, individual diastereomers and diastereomeric mixtures. Described herein are enantiomerically enriched compounds (e.g., a compound resolved to an enantiomeric excess of 60%, 70%, 80%, 85%, 90%, 95%, 99% or greater). All such isomeric forms of these compounds are expressly included in the present invention. The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts. For example a compound can be resolved to an enantiomeric excess (e.g., 60%, 70%, 80%, 85%, 90%, 95%, 99% or greater) via formation of diasteromeric salts, e.g. with a chiral base, e.g., (+) or (−) a-methylbenzylamine, or via high performance liquid chromatography using a chiral column. In some embodiments a product is purified directly on a chiral column to provide enantiomerically enriched compound.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer. ee=(90-10)/100=80%. Thus, a composition containing 90% of one enantiomer and 10% of the other enantiomer is said to have an enantiomeric excess of 80%.

The term “alkyl,” as used herein, refers to a monovalent saturated, straight- or branched-chain hydrocarbon such as a straight or branched group of 1-12, 1-10, or 1-6 carbon atoms, referred to herein as C₁-C₁₂ alkyl, C₁-C₁₀ alkyl, and C₁-C₆ alkyl, respectively.

Examples of alkyl groups include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The terms “alkenyl” and “alkynyl” are art-recognized and refer to unsaturated aliphatic groups analogous in length and possible substitution to the alkyls described above, but that contain at least one double or triple bond, respectively. Exemplary alkenyl groups include, but are not limited to, —CH═CH₂ and —CH₂CH═CH₂.

The term “alkylene” refers to the diradical of an alkyl group.

The terms “alkenylene” and “alkynylene” refer to the diradicals of an alkenyl and an alkynyl group, respectively.

The term “methylene unit” refers to a divalent —CH₂— group present in an alkyl, alkenyl, alkynyl, alkylene, alkenylene, or alkynylene moiety.

The term “carbocyclic ring system”, as used herein, means a monocyclic, or fused, spiro-fused, and/or bridged bicyclic or polycyclic hydrocarbon ring system, wherein each ring is either completely saturated or contains one or more units of unsaturation, but where no ring is aromatic.

The term “carbocyclyl” refers to a radical of a carbocyclic ring system.

Representative carbocyclyl groups include cycloalkyl groups (e.g., cyclopentyl, cyclobutyl, cyclopentyl, cyclohexyl and the like), and cycloalkenyl groups (e.g., cyclopentenyl, cyclohexenyl, cyclopentadienyl, and the like).

The term “aromatic ring system” is art-recognized and refers to a monocyclic, bicyclic or polycyclic hydrocarbon ring system, wherein at least one ring is aromatic.

The term “aryl” refers to a radical of an aromatic ring system. Representative aryl groups include fully aromatic ring systems, such as phenyl, naphthyl, and anthracenyl, and ring systems where an aromatic carbon ring is fused to one or more non-aromatic carbon rings, such as indanyl, phthalimidyl, naphthimidyl, or tetrahydronaphthyl, and the like.

The term “heteroalkyl” refers to an “alkyl” moiety wherein at least one of the carbone molecules has been replaced with a heteroatom such as O, S, or N.

The term “heteroaromatic ring system” is art-recognized and refers to monocyclic, bicyclic or polycyclic ring system wherein at least one ring is both aromatic and comprises a heteroatom; and wherein no other rings are heterocyclyl (as defined below). In certain instances, a ring which is aromatic and comprises a heteroatom contains 1, 2, 3, or 4 independently selected ring heteroatoms in such ring.

The term “heteroaryl” refers to a radical of a heteroaromatic ring system.

Representative heteroaryl groups include ring systems where (i) each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl; (ii) each ring is aromatic or carbocyclyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3(4H)-one, 5,6,7,8-tetrahydroquinolinyl and 5,6,7,8-tetrahydroisoquinolinyl; and (iii) each ring is aromatic or carbocyclyl, and at least one aromatic ring shares a bridgehead heteroatom with another aromatic ring, e.g., 4H-quinolizinyl. In certain embodiments, the heteroaryl is a monocyclic or bicyclic ring, wherein each of said rings contains 5 or 6 ring atoms where 1, 2, 3, or 4 of said ring atoms are a heteroatom independently selected from N, O, and S.

The term “heterocyclic ring system” refers to monocyclic, or fused, spiro-fused, and/or bridged bicyclic and polycyclic ring systems where at least one ring is saturated or partially unsaturated (but not aromatic) and comprises a heteroatom. A heterocyclic ring system can be attached to its pendant group at any heteroatom or carbon atom that results in a stable structure and any of the ring atoms can be optionally substituted.

The term “heterocyclyl” refers to a radical of a heterocyclic ring system.

Representative heterocyclyls include ring systems in which (i) every ring is non-aromatic and at least one ring comprises a heteroatom, e.g., tetrahydrofuranyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl; (ii) at least one ring is non-aromatic and comprises a heteroatom and at least one other ring is an aromatic carbon ring, e.g., 1,2,3,4-tetrahydroquinolinyl, 1,2,3,4-tetrahydroisoquinolinyl; and (iii) at least one ring is non-aromatic and comprises a heteroatom and at least one other ring is aromatic and comprises a heteroatom, e.g., 3,4-dihydro-1H-pyrano[4,3-c]pyridine, and 1,2,3,4-tetrahydro-2,6-naphthyridine. In certain embodiments, the heterocyclyl is a monocyclic or bicyclic ring, wherein each of said rings contains 3-7 ring atoms where 1, 2, 3, or 4 of said ring atoms are a heteroatom independently selected from N, O, and S.

The term “saturated heterocyclyl” refers to a radical of heterocyclic ring system wherein every ring is saturated, e.g., tetrahydrofuran, tetrahydro-2H-pyran, pyrrolidine, piperidine and piperazine.

“Partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

The term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds.

The term “stable”, as used herein, refers to compounds that are not substantially altered when subjected to conditions to allow for their production, detection, and, in certain embodiments, their recovery, purification, and use for one or more of the purposes disclosed herein.

As used herein, the definition of each expression, e.g., alkyl, m, n, etc., when it occurs more than once in any structure, is intended to be independent of its definition elsewhere in the same structure.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds.

The term “stable”, as used herein, refers to compounds that are not substantially altered when subjected to conditions to allow for their production, detection, and, in certain embodiments, their recovery, purification, and use for one or more of the purposes disclosed herein.

### Pattern Recognition Receptors

The disclosure presented herein features methods for the activation and induction of PRR expression (e.g., STING expression) in a subject, e.g., a subject with a proliferative disease (e.g., cancer). Pattern recognition receptors (PRRs) are a broad class of proteins which recognize pathogen-associated molecular patterns (PAMPs) conserved within pathogenic invaders. PAMPs are typically products of biosynthetic pathways that are essential to the survival and/or infectivity of the pathogen, e.g., lipopolysaccharides, glycoproteins, and nucleic acids. Recognition of PAMPs by their cognate PRRs activates signaling pathways that result in the production of immune defense factors such as pro-inflammatory and anti-inflammatory cytokines, type I interferons (IFN-α, IFN-β), and/or interferon stimulated genes (ISGs). It is well known that induction of innate immune signaling also results in the activation of T cell responses as well as the induction of adaptive immunity. These downstream immune effects are essential for clearance of the virus through apoptosis and killing of infected cells through cytotoxic T lymphocytes and other defense mechanisms. It is also well known that interferons act on ISRE (interferon response elements) that can trigger the production of ISGs, which play an important role in antiviral cellular defense.

The stimulator of interferon genes (STING) is a cytosolic microbial-derived DNA sensor that has been shown to be particularly sensitive to double-stranded DNA and cyclic dinucleotides (e.g., cyclic di-GMP) (Burdette, D. L. and Vance, R. E. (2013) Nat Immunol 14:19-26). Two molecules of STING form a homodimer mediated by an α-helix present in the C-terminal dimerization domain, and molecular binding studies have revealed that each STING dimer binds one molecule of microbial nucleic acids, e.g., DNA or a cyclic dinucleotide. Upon ligand binding, STING activates the innate immune response through interaction with RIG-I and IPS-1, resulting in interferon production (e.g., IFN-α and IFN-β) and other downstream signaling events. Since its discovery, STING has been shown to function as a critical sensor of viruses (e.g., adenovirus, herpes simplex virus, hepatitis B virus, vesicular stomatitis virus, hepatitis C virus), bacteria (e.g., Listeria monocytogenes, Legionella pneumopholia, Mycobacterium tuberculosis) and protozoa (Plasmodium falciparum, Plasmodium berghei). In addition, STING has been shown to play a major role in the innate immune response against tumor antigens, driving dendritic cell activation and subsequent T cell priming in several cancers (Woo, S. R. et al. Trends in Immunol (2015) 36:250-256).

Another class of PRRs includes RIG-I, which is the founding member of a family of PRRs termed RIG-I-like receptors (RLRs) that primarily detect RNA derived from foreign sources. It is a critical sensor of microbial infection (e.g., viral infection) in most cells and is constitutively expressed at low levels in the cytosol. After ligand binding, the expression of RIG-I is rapidly enhanced, leading to increased RIG-I concentrations in the cell (Jensen, S. and Thomsen, A. R. J Virol (2012) 86:2900-2910; Yoneyama M. et al. Nat Immunol (2004) 5:730-737). RIG-I is an ATP-dependent helicase containing a central DExD/H box ATPase domain and tandem N-terminal caspase-recruiting domains (CARDs) that mediate downstream signaling. The C-terminus of RIG-I comprises an ssRNA/dsRNA-binding domain that when unbound acts to silence CARD function at the N-terminus. Without wishing to be bound by theory, it is believed that upon recognition of target RNA structures, two N-terminal CARDs are exposed, allowing for interaction with the CARD of a downstream binding partner, IFN-β promoter stimulator 1 (IPS-1), also known as mitochondrial antiviral signaling molecule (MAVS) and CARDIF. This interaction in turn triggers further downstream signaling, such as induction of IRF3, IRF7, NF-κB, IFNs, and cytokine production that results in the initiation of the host immune response.

Other RLRs are homologous to RIG-I and function in a similar manner, including MDA5, LGP2, and RNase L. MDA5 is highly homologous to RIG-I, and has been shown to be crucial for triggering a cytokine response upon infection with picornaviruses (e.g., encephalomyocarditis virus (EMCV), Theiler's virus, and Mengo virus), Sendai virus, rabies virus, West Nile virus, rabies virus, rotavirus, murine hepatitis virus, and murine norovirus. LPG2 lacks a CARD domain found in RIG-I and MDA5, which is responsible for direct interaction with IPS-1 to initiate downstream signaling. As such, LPG2 is believed to behave as a modulator of the innate immune response in conjunction with other CARD-bearing RLRs such as RIG-I and MDA5.

Another class of PRRs encompasses the nucleotide-binding and oligomerization domain (NOD)-like receptors, or NLR family (Caruso, R. et al, Immunity (2014) 41:898-908), which includes the microbial sensor NOD2. NOD2 is composed of an N-terminal CARD, a centrally-located nucleotide-binding oligomerization domain, and a C-terminal leucine rich repeat domain that is responsible for binding microbial PAMPs, such as bacterial peptidoglycan fragments and microbial nucleic acids. Ligand binding activates NOD2 and is believed to drive interaction with the CARD-containing kinase RIPK2, which in turn activates a number of downstream proteins including NF-κB, MAPK, IRF7, and IRF3, the latter of which results in the induction of type 1 interferons. NOD2 is expressed in a diverse set of cell types, including macrophages, dendritic cells, paneth cells, epithelial cells (e.g., lung epithelial cells, intestinal epithelia), and osteoblasts. NOD2 has been established as a sensor of infection by variety of pathogenic invaders, such as protozoa (e.g., Toxoplasma gondii and Plasmodium berghei), bacteria (e.g., Bacillus anthracis, Borrelia burgdorferi, Burkholderia pseudomallei, Helicobacter hepaticus, Legionella pneumophilia, Mycobacterium tuberculosis, Propionibacterium acne, Porphyromonas gingivalis, Salmonella enterica, and Streptococcus pneumonia), and viruses (e.g., respiratory syncytial virus and murine norovirus-1) (Moreira, L. O. and Zamboni, D. S. Front Immunol (2012) 3:1-12). Recent work has shown that mutation of NOD2 may contribute to inflammatory diseases such as Crohn's disease, resulting in an aberrant inflammatory response upon stimulation.

### Compounds

The present disclosure features compounds and methods for the induction of PRR expression (e.g., STING expression) in a subject (e.g., a subject with a proliferative disease, e.g., a cancer), comprising administration of a compound or composition described herein or a prodrug or pharmaceutically acceptable salt thereof.

In an embodiment, a compound or composition described herein a in the form of a pharmaceutically acceptable salt. Exemplary salts are described herein, such as ammonium salts. In some embodiments, the compound is a mono-salt.

A compound described herein is a small molecule nucleic acid hybrid compound that combines both antiviral and immune modulating activities. The latter activity mediates, for example, controlled apoptosis of virus-infected hepatocytes via stimulation of the innate immune response, similar to what is also achieved by IFN-α therapy in patients suffering from a viral infection.

A composition described herein is a mixture of small molecule nucleic acid hybrid compounds that combine both antiviral and immune modulating activities. The latter activity mediates, for example, controlled apoptosis of virus-infected hepatocytes via stimulation of the innate immune response, similar to what is also achieved by IFN-α therapy in patients suffering from a viral infection.

Without wishing to be bound by theory, the mechanism of action of a compound or composition described herein may be dissected into two components. The first component entails the host immune stimulating activity of a compound or composition described herein, which may induce endogenous IFNs via the activation of a PRR, e.g., RIG-I, NOD2, and STING. Activation may occur by binding of a compound or composition described herein to the nucleotide binding domain of a PRR (e.g., STING), as described previously, and may further result in the induction of PRR expression (e.g., STING expression).

The second component of the mechanism of action of a compound or composition described herein involves its direct antiviral activity, which inhibits the synthesis of viral nucleic acids by steric blockage of the viral polymerase. The block may be achieved by interaction of a compound or composition described herein with a PRR (e.g., STING) as described earlier that then in turn may prevent the polymerase enzyme from engaging with the nucleic acid template for replication (e.g., viral-derived RNA). In some embodiments, a compound or composition described herein directly engages with a PRR (e.g., STING). In some embodiments, a compound or composition described herein directly engages with a PRR (e.g., STING) and induces a downstream pathway (e.g., IFN signaling).

The compounds provided herein may contain one or more asymmetric centers and thus occur as racemates and racemic mixtures, single enantiomers, individual diastereomers, and diastereomeric mixtures. All such isomeric forms of these compounds are expressly included within the scope. Unless otherwise indicated when a compound is named or depicted by a structure without specifying the stereochemistry and has one or more chiral centers, it is understood to represent all possible stereoisomers of the compound. The compounds provided herewith may also contain linkages (e.g., carbon-carbon bonds, phosphorus-oxygen bonds, or phosphorus-sulfur bonds) or substituents that can restrict bond rotation, e.g. restriction resulting from the presence of a ring or double bond.

In some embodiments, the method described herein comprises administration of a compound or composition described herein or a pharmaceutically acceptable salt thereof.

In some embodiments, a compound or composition described herein comprises an isomer (e.g., an Rp-isomer or Sp isomer) or a mixture of isomers (e.g., Rp-isomers or Sp isomers) of a compound or composition described herein.

In one aspect, the invention features a compound of Formula (I):

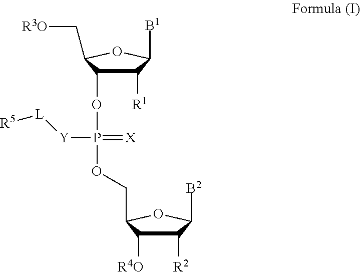

or a pharmaceutically acceptable salt, wherein:

each of B¹ and B² is independently a purinyl nucleobase or pyrimidinyl nucleobase;

X is O or S;

Y is O, S, or NR⁶;

L is absent, C₁-C₆ alkyl or C₁-C₆ heteroalkyl, wherein each C₁-C₆ alkyl and C₁-C₆ heteroalkyl is optionally substituted with R⁷;

each of R¹ and R² is independently hydrogen, halo, —CN, C₁-C₂₀ alkyl (e.g., C₁-C₆ alkyl), or OR⁸, provided that at least one of R¹ and R² is halo, O—C₁-C₂₀-alkenyl, or O—C₁-C₂₀-alkynyl or R¹ is hydrogen;

each of R³ and R⁴ is independently hydrogen or C₁-C₂₀ alkyl (e.g., C₁-C₆ alkyl).

R⁵ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹;

R⁶ is hydrogen or C₁-C₂₀ alkyl (e.g., C₁-C₆ alkyl);

R⁷ is halo, —CN, C₁-C₂₀ alkyl (e.g., C₁-C₆ alkyl), OR⁸, oxo, cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

R⁸ is hydrogen, C₁-C₂₀ alkynyl (e.g., C₁-C₆ alkynyl), C₁-C₂₀ alkenyl (e.g., C₁-C₆ alkenyl), cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

each R⁹ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R¹⁰; and

each R¹⁰ is independently C₁-C₂₀ alkyl, halo, —CN, OH, O—C₁-C₂₀ alkyl, O—C₁-C₂₀ heteroalkyl, O-aryl, or O-heteroaryl.

In some embodiments, at least one of B¹ or B² is a purinyl nucleobase. In some embodiments, each of B¹ or B² is independently a purinyl nucleobase. In some embodiments, B¹ is a purinyl nucleobase. In some embodiments, B² is a pyrimidinyl nucleobase. In some embodiments, B¹ is a purinyl nucleobase and B² is a pyrimidinyl nucleobase.

In some embodiments, each of B¹ or B² is selected from a naturally occurring nucleobase or a modified nucleobase. In some embodiments, each of B¹ or B² is selected from adenosinyl, guanosinyl, cytosinyl, thyminyl, uracilyl, 5′-methylcytosinyl, 5′-fluorouracilyl, 5′-propynyluracilyl, and 7-deazaadenosinyl. In some embodiments, each of B¹ or B² is selected from:

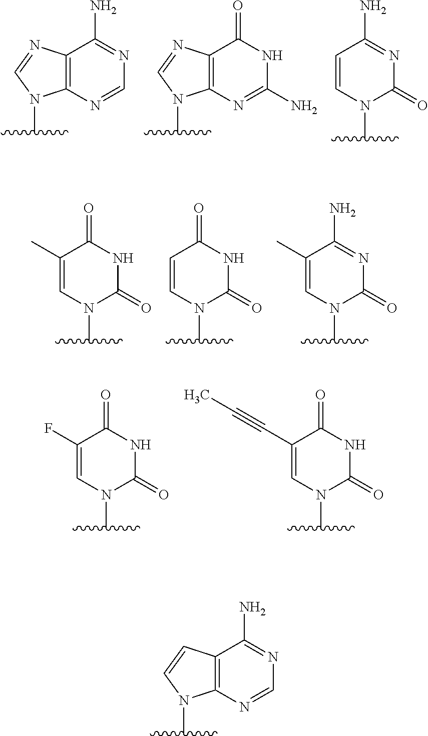

wherein “ [symbol] ” indicates the linkage of the nucleobase to the ribonse ring.

In some embodiments, one of B¹ or B² is selected from a naturally occurring nucleobase and the other of B¹ or B² is a modified nucleobase. In some embodiments, one of B¹ or B² is adenosinyl, guanosinyl, thyminyl, cytosinyl, or uracilyl, and the other of B¹ or B² is 5′-methylcytosinyl, 5′-fluorouracilyl, 5′-propynyluracilyl, or 7-deazaadenosinyl.

In some embodiments, each of R¹ and R² is independently hydrogen, fluorine, C₁-C₂₀ alkyl (e.g., C₁-C₆ alkyl), C₁-C₂₀ alkenyl (e.g., C₁-C₆ alkenyl), or O—C₁-C₂₀ alkynyl (e.g., C₁-C₆ alkynyl).

In some embodiments, each of R¹ and R² is independently fluorine.

In some embodiments, the compound is a compound of Formula (II):

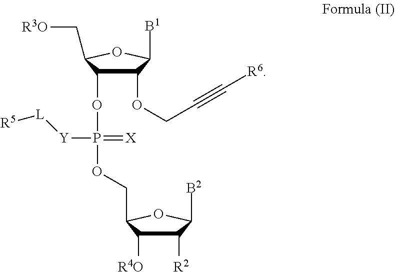

In some embodiments, R⁶ is hydrogen, C₁-C₂₀ alkyl (e.g., C₁-C₆ substituted or unsubstituted alkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹.

In some embodiments, the compound is selected from the following:

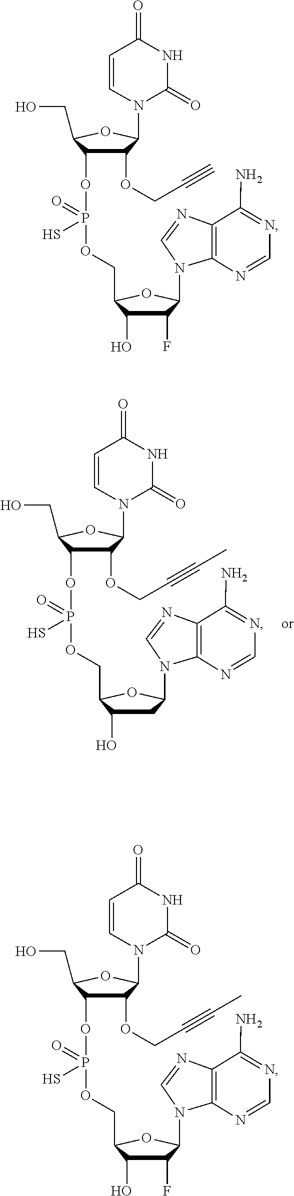

or a pharmaceutically acceptable salt thereof.

In some embodiments, the compound is selected from:

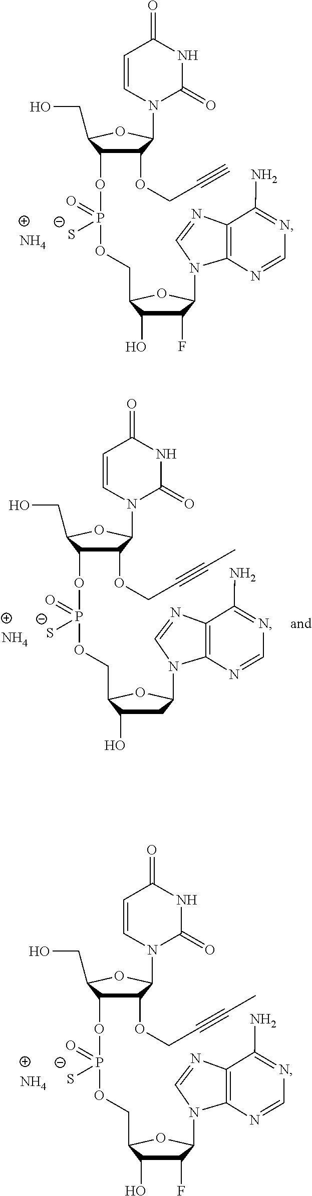

or a pharmaceutically acceptable salt thereof.

In some embodiments, the compound is selected from:

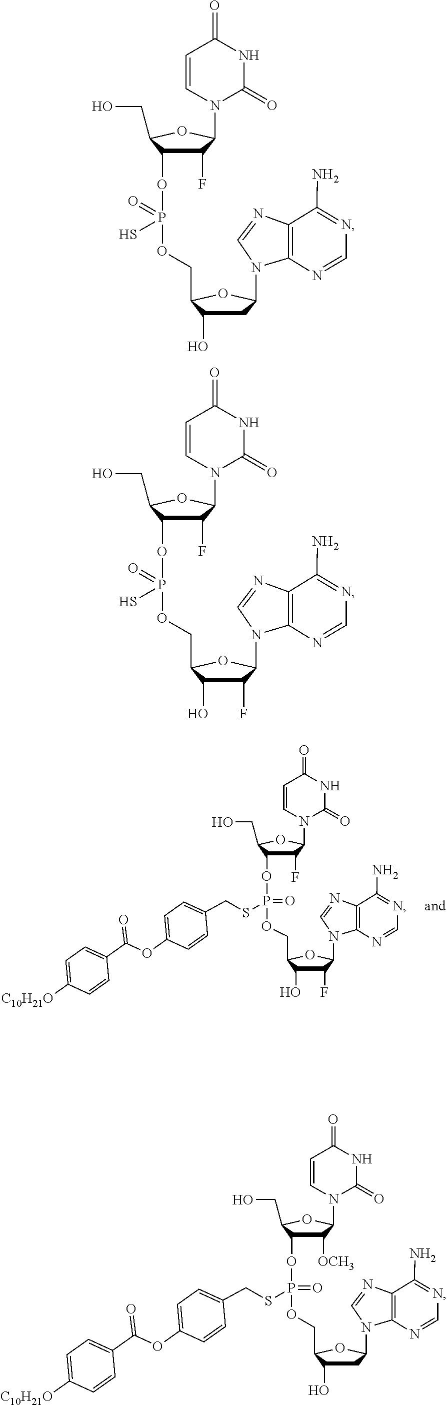

or a pharmaceutically acceptable salt thereof.

In some embodiments, the compound is selected from:

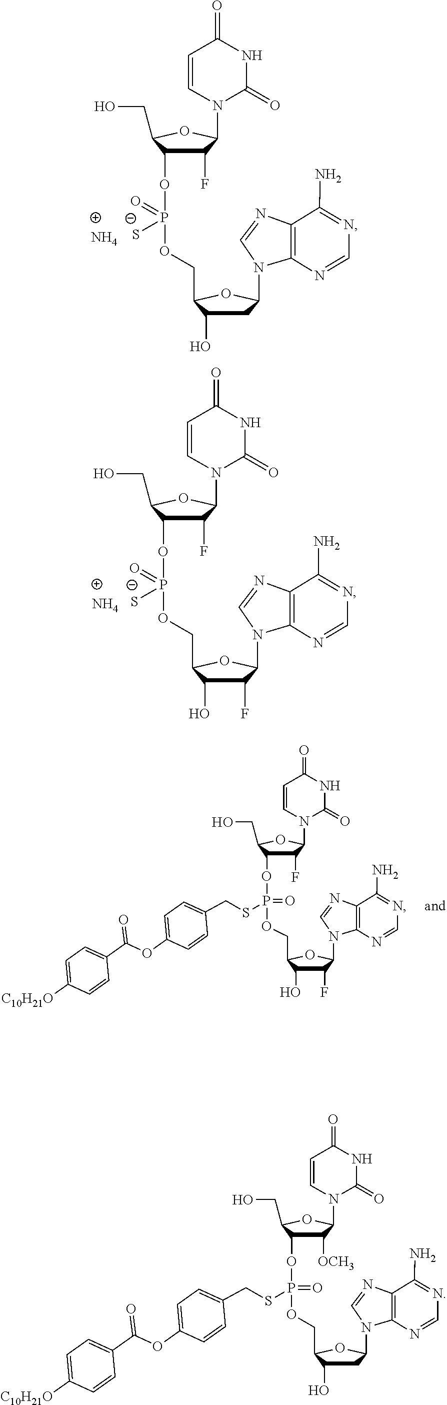

In some embodiments, the compound is selected from:

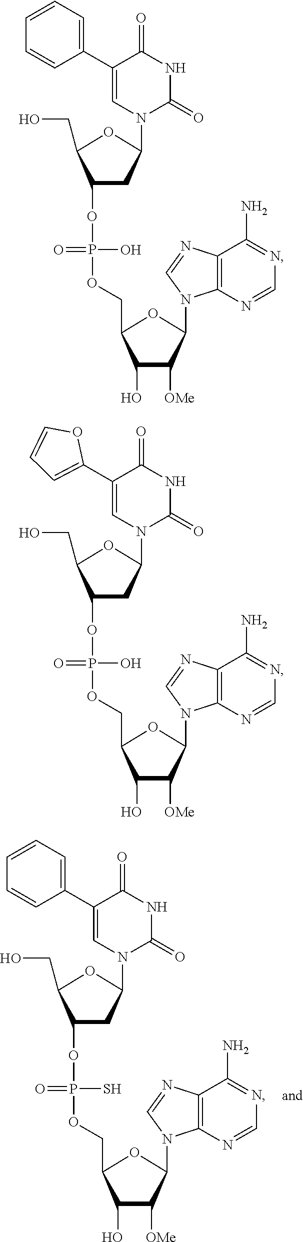

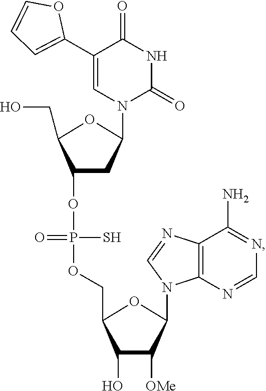

or a pharmaceutically acceptable salt thereof.

In some embodiments, the compound is selected from:

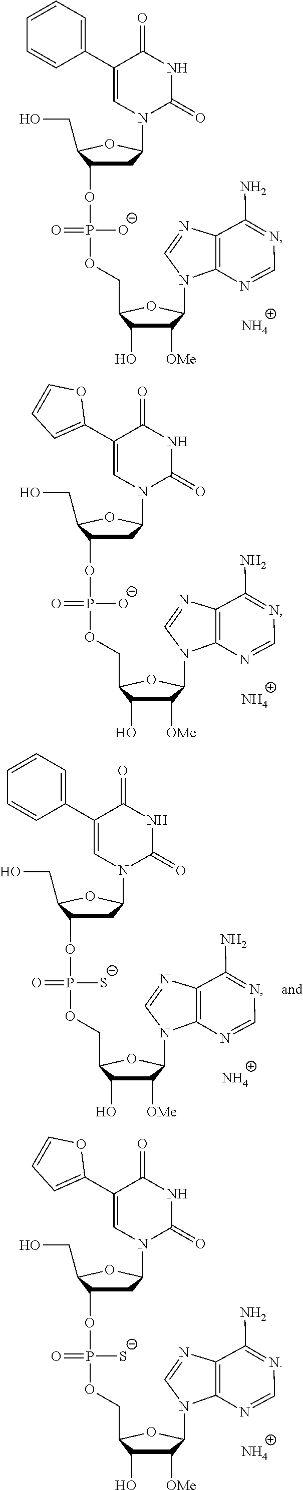

In one aspect, the invention describes herein a composition comprising compounds of Formula (III-a) or (III-b):

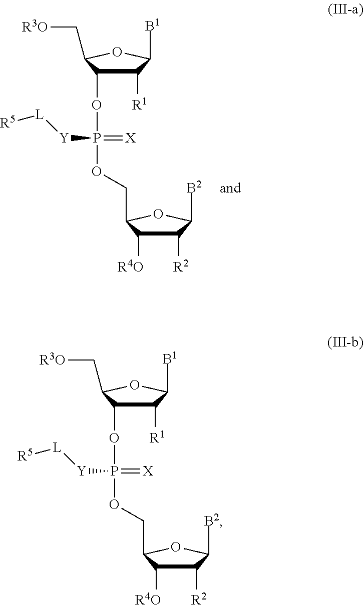

or pharmaceutically acceptable salts thereof, wherein the composition is an optically enriched mixture of Formula (III-a) or (III-b).

In some embodiments, the composition is an optically enriched mixture of a compound of Formula (III-a) or (III-b).

In some embodiments, the composition comprises a compound of Formula (III-a) or (III-b) in an enantiomeric excess of 90%.

In one aspect, the invention features a compound of Formula (IV):

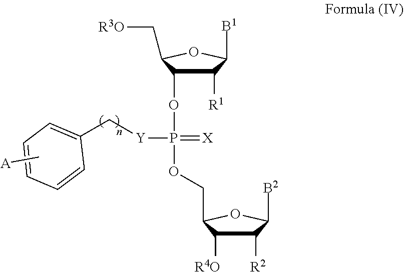

or a pharmaceutically acceptable salt or stereoisomer thereof, wherein:

each of B¹ and B² is independently a purinyl nucleobase or pyrimidinyl nucleobase;

X is O or S;

Y is O, S, or NR⁵;

n is 1, 2, or 3;

each of R¹ and R² is independently hydrogen, —CN, C₁-C₂₀ alkyl (e.g., C₁-C₆ alkyl), or OR⁶;

each of R³ and R⁴ is independently hydrogen or C₁-C₂₀ alkyl (e.g., C₁-C₆ alkyl);

R⁵ is hydrogen or C₁-C₂₀ alkyl (e.g., C₁-C₆ alkyl);

R⁶ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁷;

each R⁷ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R⁸;

each R⁸ is independently C₁-C₂₀ alkyl, halo, —CN, OH, O—C₁-C₂₀ alkyl, O—C₁-C₂₀ heteroalkyl, O-aryl, or O-heteroaryl; and

A is OC(O)—C₆-C₂₀ alkyl or OC(O)-aryl, wherein aryl is optionally substituted with C₆-C₂₀ alkyl, O—C₆-C₂₀ alkyl or C₁-C₆—O—C₆-C₂₀ alkyl.

In some embodiments, each of R¹ and R² is independently hydrogen or O—C₁-C₂₀ alkyl.

In some embodiments, A is OC(O)—C₆-C₂₀ alkyl or OC(O)-aryl, wherein aryl is substituted with C₆-C₂₀ alkyl, O—C₆-C₂₀ alkyl or C₁-C₆—O—C₆-C₂₀ alkyl.

In some embodiments, each of R³ and R⁴ is independently hydrogen.

In some embodiments, R¹ is O—C₁-C₂₀ alkyl and R² is hydrogen.

In some embodiments, the compound of Formula (IV) is selected from:

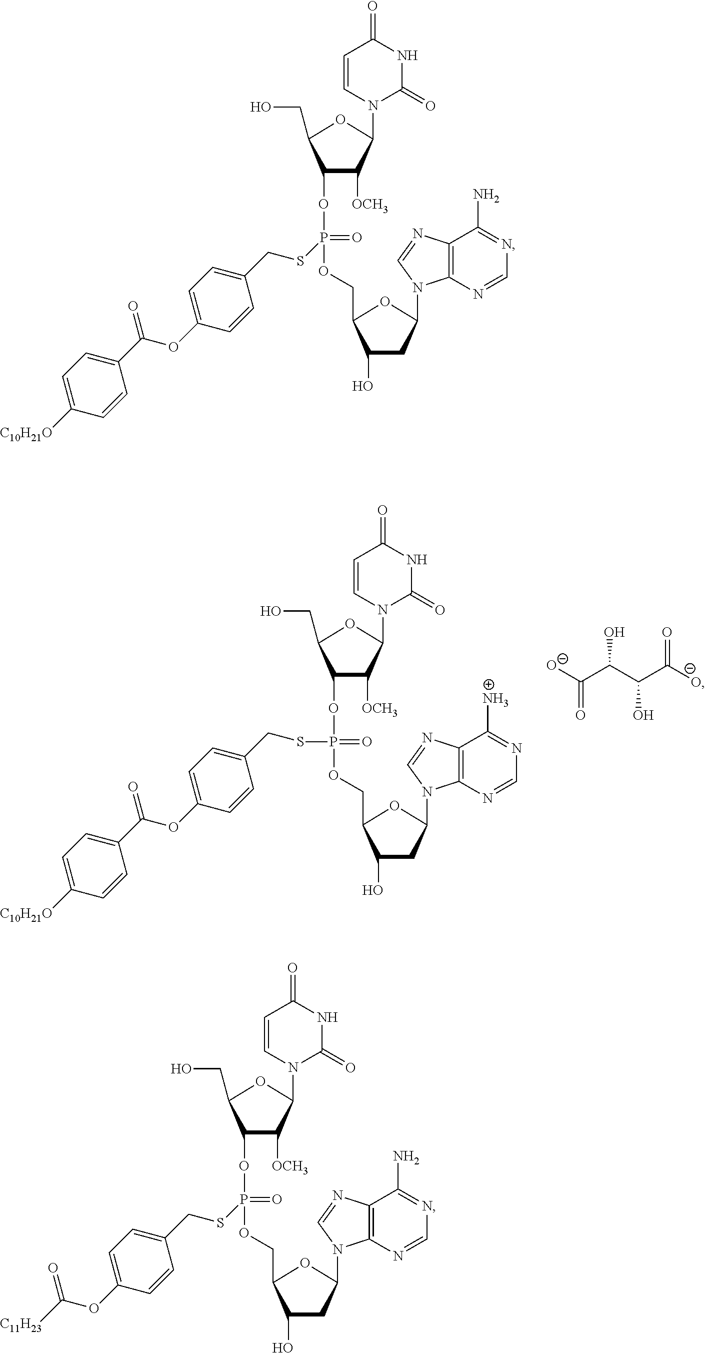

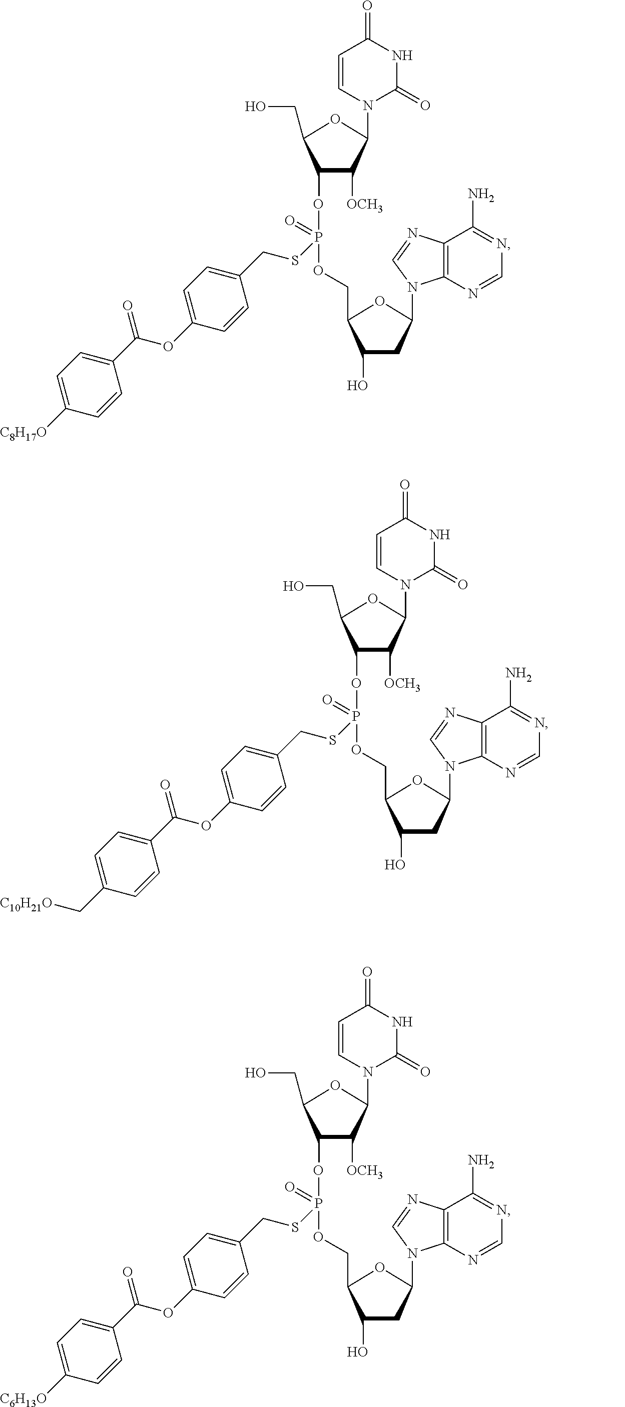

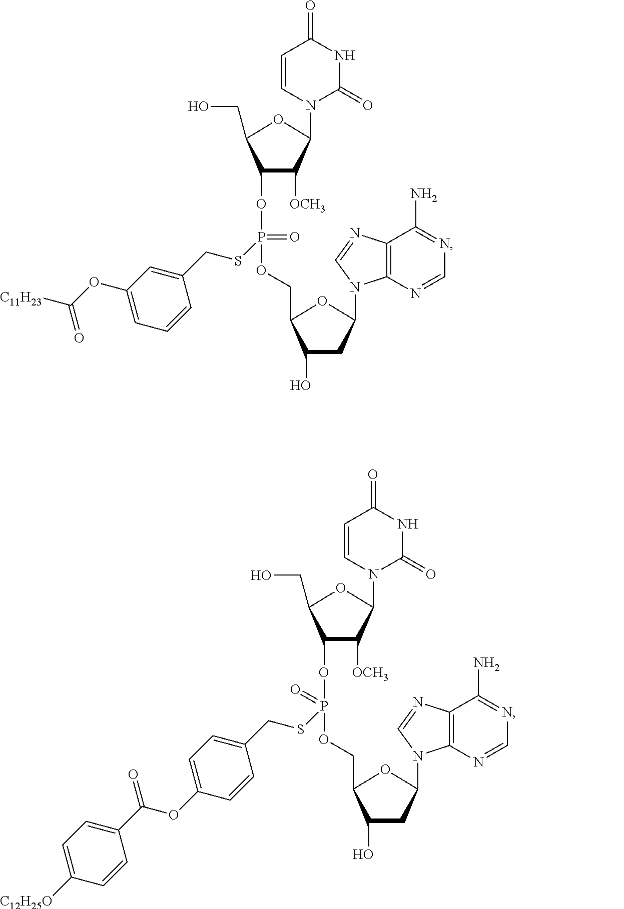

or a pharmaceutically acceptable salt thereof.

In another aspect, the present invention features a composition comprising a compound of Formula (V-a) or (V-b):

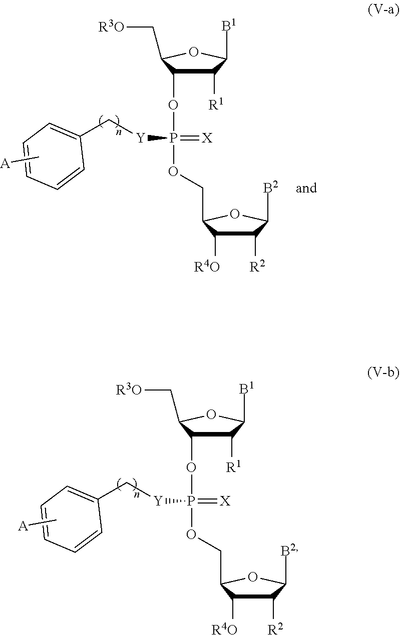

or a pharmaceutically acceptable salt thereof, wherein the composition is an optically enriched mixture of Formula (V-a) or (V-b).

In some embodiments, the composition is an optically enriched mixture of a compound of Formula (V-a) or (V-b).

In some embodiments, the composition comprises a compound of Formula (V-a) or (V-b) in an enantiomeric excess of 90%.

In some embodiments, the composition comprises a compound selected from:

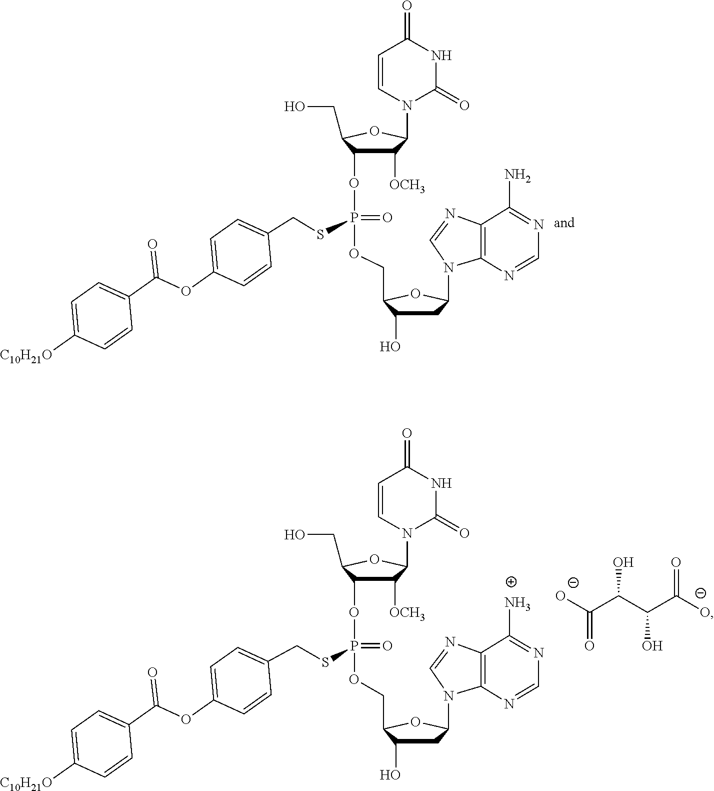

or a pharmaceutically acceptable salt thereof.

### Methods of Use

The present disclosure relates to methods for inducing the expression of a PRR (e.g., STING) in a subject through administration of a compound or composition described herein or a pharmaceutically acceptable salt thereof. In some embodiments, the subject may be suffering from a condition described below, e.g., a proliferative disease, e.g., a cancer.

It has been reported that many patients with advanced solid tumors show a spontaneous T cell-inflamed tumor microenvironment, which is predictive of prognosis and clinical response to immunotherapies. Recent findings suggest the STING pathway of cytosolic DNA sensing is an important innate immune sensing mechanism driving type I IFN production in the tumor context. Knowledge of this pathway is guiding the further development of novel immunotherapeutic strategies.

It has been reported that in early-stage colorectal cancer, the presence of activated CD8+ T cells within the tumor microenvironment significant positive prognostic outcome.

Patients with other solid tumor histology also appear to have a spontaneous T cell infiltrate that may have similar positive prognostic value. These include breast cancer, renal cell carcinoma, melanoma, ovarian cancer, and gastrointestinal tumors. It is believed that T cell infiltrate includes tumor antigen-specific T cells that have been activated spontaneously in response to the growing tumor, perhaps through immune surveillance mechanisms. This attempted host immune response, even if it does not eliminate the tumor completely, is thought to delay tumor progression and thus yield improved clinical outcome. Furthermore, the innate immune mechanisms can lead to adaptive T cell response against tumor antigens even in the absence of exogenous infection. In this regard, human cancer gene expression profiling studies reveal an association between a type I IFN signature, T cell infiltration, and clinical outcome. Thus, innate immune sensing pathways that trigger type I IFN production might represent crucial intermediate mechanistic step. In gene expression profiling of melanoma, two major subsets of tumor microenvironment have been found that represent either the presence or absence of a transcriptional profile indicative of T cell infiltrate. In fact, CD8+ T cells, macrophages, as well as of some B cells and plasma cells in these lesions in melanoma metastases is similar to the phenotype described in early-stage colon cancer and other tumors in which activated T cells have been associated with favorable prognosis. CD8+ T cells were required for the up-regulation of all immune factors within the tumor micro-environment. Studies indicate that IFN production is necessary for optimal T cell priming against tumor antigens. There are many PRRs that trigger IFN-β production by host DCs in response to a growing tumor in vivo including STING. STING is an adapter protein that is activated by cyclic dinucleotides generated by cyclic GMP-AMP synthase (cGAS), which in turn is directly activated by cytosolic DNA. Activated STING forms aggregates, activates TBK1, which in turn phosphorylates interferon regulatory factor 3 (IRF3) that directly contributes to type I IFN gene transcription. This pathway has been implicated in the sensing of DNA viruses, and also in selected autoimmune models. Moreover, activating mutations of STING have recently been identified in human patients with a vasculitis/pulmonary inflammation syndrome that is characterized by increased type I IFN production. Mechanistic studies using mouse transplantable tumor models revealed that STING-knockout mice, and IRF3-knockout mice showed defective spontaneous T cell priming against tumor antigens in vivo, and rejection of immunogenic tumors was ablated. Similarly, tumor-derived DNA was found within the cytosol of a major population of tumor-infiltrating DCs, and this was associated with STING pathway activation and IFN-β production. Therefore, the host STING pathway appears to be an important innate immune sensing pathway that detects the presence of a tumor and to drive DC activation and subsequent T cell priming against tumor-associated antigens in vivo. A functional role for the STING pathway in vivo has also been reported in other mouse-tumor systems. An inducible glioma model was shown to result in induction of a type I IFN gene signature as part of the host response. This induction was substantially reduced in STING-knockout mice, and tumors grew more aggressively, leading to shorter mouse survival. Exogenous delivery of cyclic dinucleotides as STING agonists exerted a therapeutic effect in vivo. A crucial role for host type I IFNs and the host STING pathway was also confirmed in the B16.OVA and EL4.OVA models in response to cryo-ablation.

Interestingly, the mechanisms involved paralleled what was observed in the Bm12 mouse model of lupus because host STING was also required for maximal production of anti-DNA antibodies. Thus, the antitumor immune response triggered in part by tumor DNA has overlap with the mechanisms involved in autoimmunity driven by extracellular DNA. A role for STING also has been explored in an inducible colon cancer model. It seems likely that the ability of a cancer in an individual patient to support STING pathway activation is linked to the spontaneous generation of a T cell-inflamed tumor microenvironment.

Because this phenotype is associated with improved prognosis of early-stage cancer patients, and also with clinical response to immunotherapies in the metastatic setting, failed STING activation may therefore represent an early functional block, and thus itself may have prognostic/predictive value as a biomarker. Second, strategies that activate or mimic the output of the host STING pathway should have immunotherapeutic potential in the clinic. In as much as non-T cell-inflamed tumors appear to lack evidence of a type I IFN transcriptional signature, strategies to promote robust innate signaling via APCs in the tumor microenvironment might facilitate improved cross-priming of tumor antigen-specific CD8+ T cells, and also augment chemokine production for subsequent oncolytic activity.

### Treatment of Cancer

Recognition of nucleic acid ligands by a PRRs such as cGAS, RIG-I and/STING stimulates the production of type I interferons (e.g., IFN-α or IFN-β), thus triggering a series of downstream signaling events that may lead to apoptosis in susceptible cells. In recent years, a connection between the induction of PRR expression and a number of cancers has been discovered. For example, RIG-I expression has been shown to be significantly downregulated in hepatocellular carcinoma, and patients exhibiting low RIG-I expression in tumors had shorter survival and poorer responses to IFN-α therapy (Hou, J. et al, Cancer Cell (2014) 25:49-63). As such, it has been suggested that the level of RIG-I expression may be useful as a biomarker for prediction of prognosis and response to immunotherapy. In other cases, induction of RIG-I expression has been shown to induce immunogenic cell death of pancreatic cancer cells, prostate cancer cells, breast cancer cells, skin cancer cells, and lung cancer cells (Duewell, P. et al, Cell Death Differ (2014) 21:1825-1837; Besch, R. et al, J Clin Invest (2009) 119:2399-2411; Kaneda, Y. Oncoimmunology (2013) 2:e23566; Li, X. Y. et al, Mol Cell Oncol (2014) 1:e968016), highlighting a new approach in immune-mediated cancer treatment.

STING is recognized as the key adapter protein in the cGAS-STING-IFN cascade, although it is also reported to be a sensor for DNA. A role for STING in the stimulation of innate immunity in response to cancer has also been identified. Recent studies have revealed the presence of tumor-derived DNA in the cytosol of certain antigen-presenting cells, such as tumor-infiltrating dendritic cells, likely generated through tumor cell stress or cell death. This tumor-derived DNA is known to activate cGAS which causes the production of cyclic nucleotides that have been shown to activate STING, resulting in production of associated type 1 interferons (Woo, S. R. et al, Immunity (2014) 41:830-842). Stimulation of STING and resulting downstream signaling pathways also likely contributes to effector T cell recruitment into the inflamed tumor microenvironment (Woo, S. R. Trends in Immunol (2015) 36:250-256). STING activation in the tumor microenvironment can induce adaptive immune response leading to anti-tumor activity. Hence, in those tumors that are STING-deficient, the described herein can still have anti-tumor activity through activation of antigen-presenting cells and dendritic cells, (APCs and DCs) and induction of adaptive immune response.

In some embodiments, the methods of inducing expression of a PRR (e.g., a PRR described herein) comprise administration of a compound or composition described herein or a pharmaceutically acceptable salt thereof to a subject suffering from cancer. In some embodiments, the methods of inducing expression of STING disclosed herein comprise administration of a compound of a compound or composition described herein or a pharmaceutically acceptable salt thereof to a subject suffering from cancer. In some embodiments, the methods of inducing expression of RIG-I disclosed herein comprise administration of a compound or composition described herein or a pharmaceutically acceptable salt thereof to a subject suffering from cancer. In some embodiments, the methods of inducing expression of NOD2 disclosed herein comprise administration of a compound or composition described herein or a pharmaceutically acceptable salt thereof to a subject suffering from cancer. In some embodiments, the cancer is selected from a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body. In some embodiments, the cancer comprises a solid tumor (e.g., a carcinoma, a sarcoma, or a lymphoma). In some embodiments, the cancer is a hepatocellular carcinoma or other cancer of the liver. In some embodiments, the cancer is a leukemia or other cancer of the blood. In some embodiments, the cancer comprises breast cancer, renal cell carcinoma, colon cancer, melanoma, ovarian cancer, head and neck squamous cell carcinoma, pancreatic cancer, prostate cancer, lung cancer, brain cancer, thyroid cancer, renal cancer, testis cancer, stomach cancer, urothelial cancer, skin cancer, cervical cancer, endometrial cancer, liver cancer, lung cancer, lymphoma or gastrointestinal stromal cancer and solid tumors. In some embodiments, the cancer cells (e.g., tumor cells) comprise specific cancer-associated antigens that induce a T-cell-mediated anti-tumor response.

In some embodiments, the methods of inducing expression of a PRR (e.g., STING) in a subject suffering from a cancer disclosed herein result in an increase in PRR expression (e.g., STING expression). In some embodiments, expression of a PRR (e.g., STING) is induced by a factor of about 1.1, about 1.2, about 1.3, about 1.4, about 1.5, about 1.6, about 1.7, about 1.8, about 1.9, about 2, about 2.5, about 3, about 4, about 5, about 7.5, about 10, about 15, about 20, about 25, about 30, about 40, about 50, about 75, about 100, about 150, about 200, about 250, about 500, about 1000, about 1500, about 2500, about 5000, about 10,000, or more. In some embodiments, induction of expression of a PRRs e.g., STING) occurs within about 5 minutes of administration of a compound or composition described herein or a pharmaceutically acceptable salt thereof. In some embodiments, induction of expression of a PRRs (e.g., STING) occurs within about 5 minutes of administration of a compound or composition described herein or a pharmaceutically acceptable salt thereof. In some embodiments, induction of expression of a PRR (e.g., STING) occurs within about 10 minutes, about 15 minutes, about 20 minutes, about 25 minutes, about 30 minutes, about 45 minutes, about 1 hour, about 1.5 hours, about 2 hours, about 3 hours, about 4 hours, about 5 hours, about 6 hours, about 7 hours, about 8 hours, about 10 hours, about 12 hours or more following administration of a compound or composition described herein or a pharmaceutically acceptable salt thereof.

It is recognized that activation of STING by compounds may lead to induction of expression of other PRRs such as RIG-I, MDA5, NOD2 etc. which may further amplify IFN production in the tumor microenvironment and prime T-cells for enhanced anti-tumor activity.

In some embodiments, the methods of inducing expression of a PRR (e.g., STING) in a subject suffering from a cancer disclosed herein result in an increase in PRR expression (e.g., STING expression). In some embodiments, expression of a PRR (e.g., STING) is induced by a factor of about 1.1, about 1.2, about 1.3, about 1.4, about 1.5, about 1.6, about 1.7, about 1.8, about 1.9, about 2, about 2.5, about 3, about 4, about 5, about 7.5, about 10, about 15, about 20, about 25, about 30, about 40, about 50, about 75, about 100, about 150, about 200, about 250, about 500, about 1000, about 1500, about 2500, about 5000, about 10,000, or more. In some embodiments, induction of expression of a PRR (e.g., STING) occurs within about 5 minutes of administration of a compound or composition described herein or a pharmaceutically acceptable salt or stereoisomer thereof. In some embodiments, induction of expression of a PRR (e.g., STING) occurs within about 10 minutes, about 15 minutes, about 20 minutes, about 25 minutes, about 30 minutes, about 45 minutes, about 1 hour, about 1.5 hours, about 2 hours, about 3 hours, about 4 hours, about 5 hours, about 6 hours, about 7 hours, about 8 hours, about 10 hours, about 12 hours or more following administration of a compound or composition described herein or a pharmaceutically acceptable salt thereof.

In one aspect, the invention describes herein a method of treating cancer in a subject, the method comprising administering to the subject an effective amount of a compound of Formula (I),

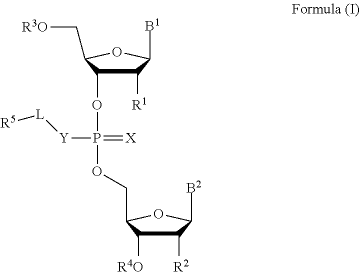

or a pharmaceutically acceptable salt, wherein:

each of B¹ and B² is independently a purinyl nucleobase or pyrimidinyl nucleobase;

X is O or S;

Y is O, S, or NR⁶;

L is absent, C₁-C₆ alkyl or C₁-C₆ heteroalkyl, wherein each C₁-C₆ alkyl and C₁-C₆ heteroalkyl is optionally substituted with R⁷;

each of R¹ and R² is independently hydrogen, halo, —CN, C₁-C₂₀ alkyl (e.g., C₁-C₆ alkyl), or OR⁸, provided that at least one of R¹ and R² is halo, O—C₁-C₂₀-alkenyl, or O—C₁-C₂₀-alkynyl or R¹ is hydrogen;

each of R³ and R⁴ is independently hydrogen or C₁-C₂₀ alkyl (e.g., C₁-C₆ alkyl).

R⁵ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹;

R⁶ is hydrogen or C₁-C₂₀ alkyl (e.g., C₁-C₆ alkyl);

R⁷ is halo, —CN, C₁-C₂₀ alkyl (e.g., C₁-C₆ alkyl), OR⁸, oxo, cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

R⁸ is hydrogen, C₁-C₂₀ alkynyl (e.g., C₁-C₆ alkynyl), C₁-C₂₀ alkenyl (e.g., C₁-C₆ alkenyl), cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

each R⁹ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R¹⁰; and

each R¹⁰ is independently C₁-C₂₀ alkyl, halo, —CN, OH, O—C₁-C₂₀ alkyl, O—C₁-C₂₀ heteroalkyl, O-aryl, or O-heteroaryl.

In some embodiments, the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

In some embodiments, the cancer is a cancer of the liver.

In some embodiments, any of the above methods within this aspect further comprise administration of an additional agent (e.g., an anticancer agent).

In some embodiments, the additional agent comprises methotrexate, 5-fluorouracil, doxorubicin, vincristine, bleomycin, vinblastine, dacarbazine, toposide, cisplatin, epirubicin, or sorafenib tosylate.

In one aspect, the invention describes herein a method of inducing the expression of a pattern recognition receptors (PRRs) for immune-modulation in a subject, the method comprising administering to the subject an effective amount of a compound of Formula (I),

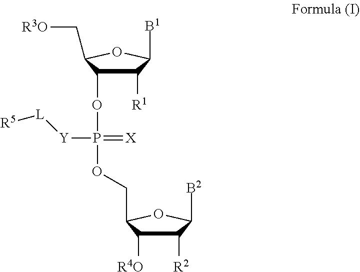

or a pharmaceutically acceptable salt, wherein:

each of B¹ and B² is independently a purinyl nucleobase or pyrimidinyl nucleobase;

X is O or S;

Y is O, S, or NR⁶;

L is absent, C₁-C₆ alkyl or C₁-C₆ heteroalkyl, wherein each C₁-C₆ alkyl and C₁-C₆ heteroalkyl is optionally substituted with R⁷;

each of R¹ and R² is independently hydrogen, halo, —CN, C₁-C₂₀ alkyl (e.g., C₁-C₆ alkyl), or OR⁸, provided that at least one of R¹ and R² is halo, O—C₁-C₂₀-alkenyl, or O—C₁-C₂₀-alkynyl or R¹ is hydrogen;

each of R³ and R⁴ is independently hydrogen or C₁-C₂₀ alkyl (e.g., C₁-C₆ alkyl).

R⁵ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹;

R⁶ is hydrogen or C₁-C₂₀ alkyl (e.g., C₁-C₆ alkyl);

R⁷ is halo, —CN, C₁-C₂₀ alkyl (e.g., C₁-C₆ alkyl), OR⁸, oxo, cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

R⁸ is hydrogen, C₁-C₂₀ alkynyl (e.g., C₁-C₆ alkynyl), C₁-C₂₀ alkenyl (e.g., C₁-C₆ alkenyl), cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

each R⁹ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R¹⁰; and

each R¹⁰ is independently C₁-C₂₀ alkyl, halo, —CN, OH, O—C₁-C₂₀ alkyl, O—C₁-C₂₀ heteroalkyl, O-aryl, or O-heteroaryl.

In one aspect, the invention describes herein a method of inducing the expression of a pattern recognition receptors for immunomodulation and inducing a therapeutic response in a subject having cancer, the method comprising administering to the subject an effective amount of a compound of Formula (I),

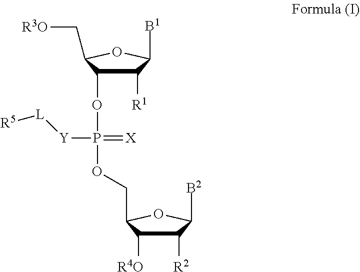

or a pharmaceutically acceptable salt, wherein:

each of B¹ and B² is independently a purinyl nucleobase or pyrimidinyl nucleobase;

X is O or S;

Y is O, S, or NR⁶;

L is absent, C₁-C₆ alkyl or C₁-C₆ heteroalkyl, wherein each C₁-C₆ alkyl and C₁-C₆ heteroalkyl is optionally substituted with R⁷;

each of R¹ and R² is independently hydrogen, halo, —CN, C₁-C₂₀ alkyl (e.g., C₁-C₆ alkyl), or OR⁸, provided that at least one of R¹ and R² is halo, O—C₁-C₂₀-alkenyl, or O—C₁-C₂₀-alkynyl or R¹ is hydrogen;

each of R³ and R⁴ is independently hydrogen or C₁-C₂₀ alkyl (e.g., C₁-C₆ alkyl).

R⁵ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹;

R⁶ is hydrogen or C₁-C₂₀ alkyl (e.g., C₁-C₆ alkyl);

R⁷ is halo, —CN, C₁-C₂₀ alkyl (e.g., C₁-C₆ alkyl), OR⁸, oxo, cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

R⁸ is hydrogen, C₁-C₂₀ alkynyl (e.g., C₁-C₆ alkynyl), C₁-C₂₀ alkenyl (e.g., C₁-C₆ alkenyl), cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;

each R⁹ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R¹⁰; and

each R¹⁰ is independently C₁-C₂₀ alkyl, halo, —CN, OH, O—C₁-C₂₀ alkyl, O—C₁-C₂₀ heteroalkyl, O-aryl, or O-heteroaryl.

In one aspect, the invention describes herein a method of treating cancer in a subject, the method comprising administering to the subject an effective amount of a composition comprising compounds of Formula (III-a) or (III-b),

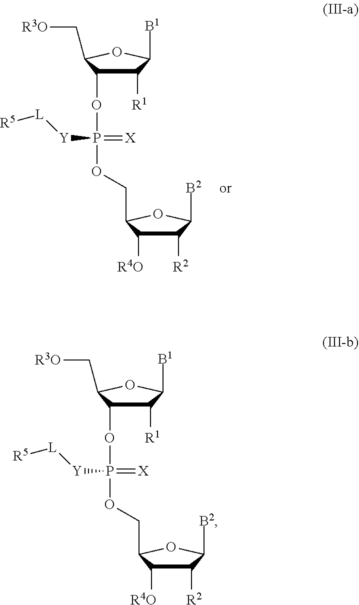

or pharmaceutically acceptable salts thereof, wherein the composition is a mixture of a compound of Formula (III-a) or (III-b).

In some embodiments, the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

In some embodiments, the cancer is a cancer of the liver.

In some embodiments, any of the above methods within this aspect further comprises administration of an additional agent (e.g., an anticancer agent).

In some embodiments, the additional agent comprises methotrexate, 5-fluorouracil, doxorubicin, vincristine, bleomycin, vinblastine, dacarbazine, toposide, cisplatin, epirubicin, or sorafenib tosylate.

In one aspect, the invention describes herein a method of inducing the expression of a pattern recognition receptors (PRRs) for immune-modulation in a subject, the method comprising administering to the subject an effective amount of a composition comprising compounds of Formula (III-a) or (III-b),

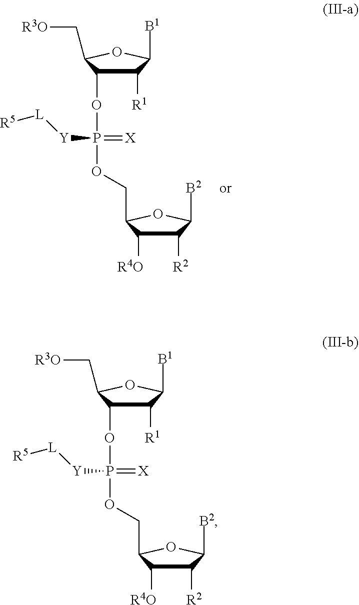

or pharmaceutically acceptable salts thereof, wherein the composition is a mixture of a compound of Formula (III-a) or (III-b).

In one aspect, the invention describes herein a method of inducing the expression of a pattern recognition receptors for immunomodulation and inducing a therapeutic response in a subject having cancer, the method comprising administering to the subject an effective amount of a composition comprising compounds of Formula (III-a) or (III-b),

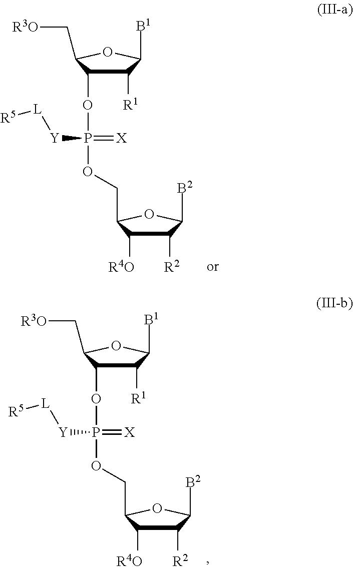

or pharmaceutically acceptable salts thereof, wherein the composition is a mixture of a compound of Formula (III-a) or (III-b).

In one aspect, the invention describes herein a method of treating cancer in a subject, the method comprising administering to the subject an effective amount of a compound of Formula (IV),

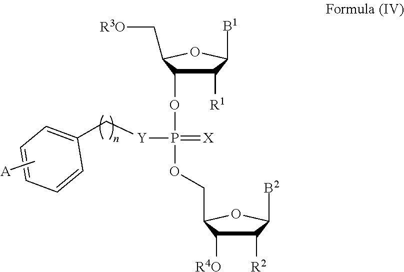

or a pharmaceutically acceptable salt or stereoisomer thereof, wherein:

each of B¹ and B² is independently a purinyl nucleobase or pyrimidinyl nucleobase;

X is O or S;

Y is O, S, or NR⁵;

n is 1, 2, or 3;

each of R¹ and R² is independently hydrogen, —CN, C₁-C₂₀ alkyl (e.g., C₁-C₆ alkyl), or OR⁶;

each of R³ and R⁴ is independently hydrogen or C₁-C₂₀ alkyl (e.g., C₁-C₆ alkyl);

R⁵ is hydrogen or C₁-C₂₀ alkyl (e.g., C₁-C₆ alkyl);

R⁶ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁷;

each R⁷ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R⁸;

each R⁸ is independently C₁-C₂₀ alkyl, halo, —CN, OH, O—C₁-C₂₀ alkyl, O—C₁-C₂₀ heteroalkyl, O-aryl, or O-heteroaryl; and

A is OC(O)—C₆-C₂₀ alkyl or OC(O)-aryl, wherein aryl is optionally substituted with C₆-C₂₀ alkyl, O—C₆-C₂₀ alkyl or C₁-C₆—O—C₆-C₂₀ alkyl.

In some embodiments, the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

In some embodiments, the cancer is a cancer of the liver.

In some embodiments, any of the above methods within this aspect further comprises administration of an additional agent (e.g., an anticancer agent).

In some embodiments, the additional agent comprises methotrexate, 5-fluorouracil, doxorubicin, vincristine, bleomycin, vinblastine, dacarbazine, toposide, cisplatin, epirubicin, or sorafenib tosylate.

In one aspect, the invention describes herein a method of inducing the expression of a pattern recognition receptors (PRRs) for immune-modulation in a subject, the method comprising administering to the subject an effective amount of a compound of Formula (IV),

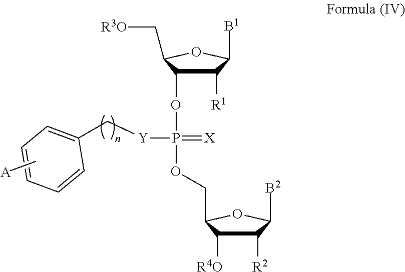

or a pharmaceutically acceptable salt or stereoisomer thereof, wherein:

each of B¹ and B² is independently a purinyl nucleobase or pyrimidinyl nucleobase;

X is O or S;

Y is O, S, or NR⁵;

n is 1, 2, or 3;

each of R¹ and R² is independently hydrogen, —CN, C₁-C₂₀ alkyl (e.g., C₁-C₆ alkyl), or OR⁶;

each of R³ and R⁴ is independently hydrogen or C₁-C₂₀ alkyl (e.g., C₁-C₆ alkyl);

R⁵ is hydrogen or C₁-C₂₀ alkyl (e.g., C₁-C₆ alkyl);

R⁶ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁷;

each R⁷ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R⁸;

each R⁸ is independently C₁-C₂₀ alkyl, halo, —CN, OH, O—C₁-C₂₀ alkyl, O—C₁-C₂₀ heteroalkyl, O-aryl, or O-heteroaryl; and

A is OC(O)—C₆-C₂₀ alkyl or OC(O)-aryl, wherein aryl is optionally substituted with C₆-C₂₀ alkyl, O—C₆-C₂₀ alkyl or C₁-C₆—O—C₆-C₂₀ alkyl.

In one aspect, the invention describes herein a method of inducing the expression of a pattern recognition receptors for immunomodulation and inducing a therapeutic response in a subject having cancer, the method comprising administering to the subject an effective amount of a compound of Formula (IV),

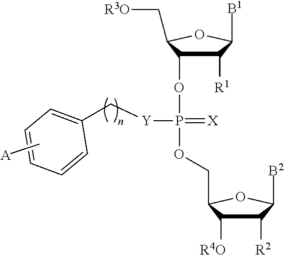

or a pharmaceutically acceptable salt or stereoisomer thereof, wherein:

each of B¹ and B² is independently a purinyl nucleobase or pyrimidinyl nucleobase;

X is O or S;

Y is O, S, or NR;

n is 1, 2, or 3;

each of R¹ and R² is independently hydrogen, —CN, C₁-C₂₀ alkyl (e.g., C₁-C₆ alkyl), or OR⁶;

each of R³ and R⁴ is independently hydrogen or C₁-C₂₀ alkyl (e.g., C₁-C₆ alkyl);

R⁵ is hydrogen or C₁-C₂₀ alkyl (e.g., C₁-C₆ alkyl);

R⁶ is hydrogen, C₁-C₂₀ alkyl (e.g., C₁-C₆ alkyl), C₁-C₂₀ heteroalkyl (e.g., C₁-C₆ heteroalkyl), cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁷;

each R⁷ is independently C₁-C₂₀ alkyl (e.g., C₁-C₆ alkyl), C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R⁸;

each R⁸ is independently C₁-C₂₀ alkyl, halo, —CN, OH, O—C₁-C₂₀ alkyl, O—C₁-C₂₀ heteroalkyl, O-aryl, or O-heteroaryl; and

A is OC(O)—C₆-C₂₀ alkyl or OC(O)-aryl, wherein aryl is optionally substituted with C₆-C₂₀ alkyl, O—C₆-C₂₀ alkyl or C₁-C₆—O—C₆-C₂₀ alkyl.

In one aspect, the invention describes herein a method of treating cancer in a subject, the method comprising administering to the subject an effective amount of a composition comprising compounds of Formula (V-a) or (V-b),

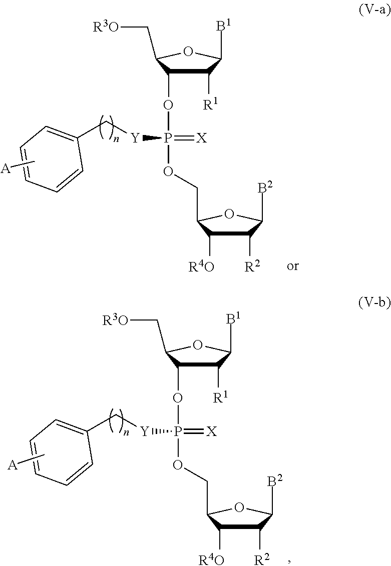

or pharmaceutically acceptable salts thereof, wherein the composition is a mixture of a compound of Formula (V-a) or (V-b).

In some embodiments, the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

In some embodiments, the cancer is a cancer of the liver.

In some embodiments, any of the above methods within this aspect further comprises administration of an additional agent (e.g., an anticancer agent).

In some embodiments, the additional agent comprises methotrexate, 5-fluorouracil, doxorubicin, vincristine, bleomycin, vinblastine, dacarbazine, toposide, cisplatin, epirubicin, or sorafenib tosylate.

In one aspect, the invention describes herein a method of inducing the expression of a pattern recognition receptors (PRRs) for immune-modulation in a subject, the method comprising administering to the subject an effective amount of a composition comprising compounds of Formula (V-a) or (V-b),

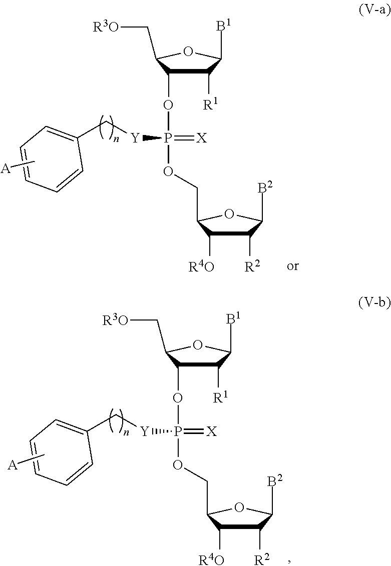

or pharmaceutically acceptable salts thereof, wherein the composition is a mixture of a compound of Formula (V-a) or (V-b).

In one aspect, the invention describes herein a method of inducing the expression of a pattern recognition receptors for immunomodulation and inducing a therapeutic response in a subject having cancer, the method comprising administering to the subject an effective amount of a composition comprising compounds of Formula (V-a) or (V-b),

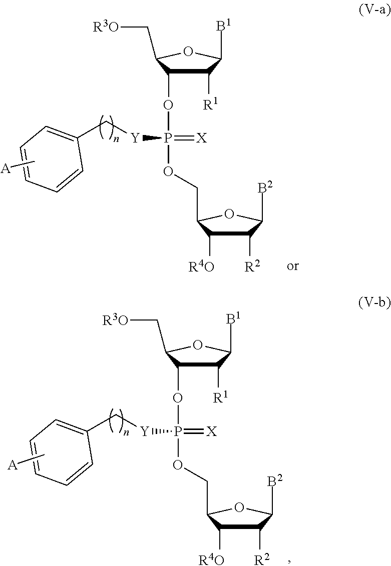

or pharmaceutically acceptable salts thereof, wherein the composition is a mixture of a compound of Formula (V-a) or (V-b).

### Pharmaceutical Compositions

The present invention features methods for inducing the expression of a PRR (e.g., STING) in a subject, the methods comprising administering a compound or composition described herein or a pharmaceutically acceptable salt thereof.

While it is possible for the compound of the present invention (e.g., a compound or composition described herein) to be administered alone, it is preferable to administer said compound as a pharmaceutical composition or formulation, where the compounds are combined with one or more pharmaceutically acceptable diluents, excipients or carriers.

The compounds according to the invention may be formulated for administration in any convenient way for use in human or veterinary medicine. In certain embodiments, the compounds included in the pharmaceutical preparation may be active itself, or may be a prodrug, e.g., capable of being converted to an active compound in a physiological setting.

Regardless of the route of administration selected, the compounds of the present invention, which may be used in a suitable hydrated form, and/or the pharmaceutical compositions of the present invention, are formulated into a pharmaceutically acceptable dosage form such as described below or by other conventional methods known to those of skill in the art.

The amount and concentration of compounds of the present invention (e.g., a compound or composition described herein) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

Thus, another aspect of the present invention provides pharmaceutically acceptable compositions comprising a therapeutically effective amount or prophylactically effective amount of a compound or composition described herein (e.g., a compound or composition described herein), formulated together with one or more pharmaceutically acceptable carriers (additives) and/or diluents. As described in detail below, the pharmaceutical compositions of the present invention may be specially formulated for administration in solid or liquid form, including those adapted for oral or parenteral administration, for example, by oral dosage, or by subcutaneous, intramuscular or intravenous injection as, for example, a sterile solution or suspension. However, in certain embodiments the subject compounds may be simply dissolved or suspended in sterile water. In certain embodiments, the pharmaceutical preparation is non-pyrogenic, i.e., does not elevate the body temperature of a patient.

The phrases “systemic administration,” “administered systemically,” “peripheral administration” and “administered peripherally” as used herein mean the administration of the compound other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, stabilizing agent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) ascorbic acid; (17) pyrogen-free water; (18) isotonic saline; (19) Ringer's solution; (20) ethyl alcohol; (21) phosphate buffer solutions; (22) cyclodextrins such as Captisol®; and (23) other non-toxic compatible substances such as antioxidants and antimicrobial agents employed in pharmaceutical formulations.

As set out above, certain embodiments of the compounds described herein may contain a basic functional group, such as an amine, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed.

Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of the compound of the present invention (e.g., a compound or composition described herein. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutically acceptable carriers, as well as wetting agents, emulsifiers, lubricants, coloring agents, release agents, coating agents, sweetening, flavoring agents, perfuming agents, preservatives, antioxidants, and other additional components may be present in an amount between about 0.001% and 99% of the composition described herein.

For example, said pharmaceutically acceptable carriers, as well as wetting agents, emulsifiers, lubricants, coloring agents, release agents, coating agents, sweetening, flavoring agents, perfuming agents, preservatives, antioxidants, and other additional components may be present from about 0.005%, about 0.01%, about 0.05%, about 0.1%, about 0.25%, about 0.5%, about 0.75%, about 1%, about 1.5%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 85%, about 90%, about 95%, or about 99% of the composition described herein.

Pharmaceutical compositions of the present invention may be in a form suitable for oral administration, e.g., a liquid or solid oral dosage form. In some embodiments, the liquid dosage form comprises a suspension, a solution, a linctus, an emulsion, a drink, an elixir, or a syrup. In some embodiments, the solid dosage form comprises a capsule, tablet, powder, dragée, or powder. The pharmaceutical composition may be in unit dosage forms suitable for single administration of precise dosages. Pharmaceutical compositions may comprise, in addition to the compound described herein (e.g., a compound or composition described herein) or a pharmaceutically acceptable salt thereof, a pharmaceutically acceptable carrier, and may optionally further comprise one or more pharmaceutically acceptable excipients, such as, for example, stabilizers (e.g., a binder, e.g., polymer, e.g., a precipitation inhibitor, diluents, binders, and lubricants.

In some embodiments, the composition described herein comprises a liquid dosage form for oral administration, e.g., a solution or suspension. In other embodiments, the composition described herein comprises a solid dosage form for oral administration capable of being directly compressed into a tablet. In addition, said tablet may include other medicinal or pharmaceutical agents, carriers, and or adjuvants. Exemplary pharmaceutical compositions include compressed tablets (e.g., directly compressed tablets), e.g., comprising a compound of the present invention (e.g., a compound or composition described herein) or a pharmaceutically acceptable salt thereof.

Formulations of the present invention include those suitable for parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about 99 percent of active ingredient, preferably from about 5 percent to about 70 percent, most preferably from about 10 percent to about 30 percent. Pharmaceutical compositions of this invention suitable for parenteral administration comprise compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a compound of the present invention (e.g., a compound or composition described herein), it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form.

Alternatively, delayed absorption of a parenterally administered form of the compound of the present invention is accomplished by dissolving or suspending compound in an oil vehicle.

In some embodiments, it may be advantageous to administer the compound of the present invention (e.g., a compound or composition described herein) in a sustained fashion. It will be appreciated that any formulation that provides a sustained absorption profile may be used. In certain embodiments, sustained absorption may be achieved by combining a compound of the present invention with other pharmaceutically acceptable ingredients, diluents, or carriers that slow its release properties into systemic circulation.

### Routes of Administration

The compounds and compositions used in the methods described herein may be administered to a subject in a variety of forms depending on the selected route of administration, as will be understood by those skilled in the art. Exemplary routes of administration of the compositions used in the methods described herein include topical, enteral, or parenteral applications. Topical applications include but are not limited to epicutaneous, inhalation, enema, eye drops, ear drops, and applications through mucous membranes in the body. Enteral applications include oral administration, rectal administration, vaginal administration, and gastric feeding tubes. Parenteral administration includes intravenous, intraarterial, intracapsular, intraorbital, intracardiac, intradermal, transtracheal, subcuticular, intraarticular, subcapsular, subarachnoid, intraspinal, epidural, intrastemal, intraperitoneal, subcutaneous, intramuscular, transepithelial, nasal, intrapulmonary, intrathecal, rectal, and topical modes of administration. Parenteral administration may be by continuous infusion over a selected period of time. In certain embodiments of the invention, a composition described herein comprising a compound or composition described herein is administered orally. In other embodiments of the invention, a composition described herein comprising a compound or composition described herein is administered parenterally (e.g., intraperitoneally). It is recognized that for treatment of solid tumors, direct injection of the compounds into the tumor may also be carried out.

For intravenous, intraperitoneal, or intrathecal delivery or direct injection, the composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

The choice of the route of administration will depend on whether a local or systemic effect is to be achieved. For example, for local effects, the composition can be formulated for topical administration and applied directly where its action is desired. For systemic, long term effects, the composition can be formulated for enteral administration and given via the digestive tract. For systemic, immediate and/or short term effects, the composition can be formulated for parenteral administration and given by routes other than through the digestive tract.

### Dosages

The compositions of the present invention are formulated into acceptable dosage forms by conventional methods known to those of skill in the art. Actual dosage levels of the active ingredients in the compositions of the present invention (e.g., a compound or composition described herein) may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the subject being treated, and like factors well known in the medical arts. A physician or veterinarian having ordinary skill in the art can readily determine and prescribe the effective amount of the composition required. For example, the physician or veterinarian can start doses of the substances of the invention employed in the composition at levels lower than that required in order to achieve the desired therapeutic effect and gradually increase the dosage until the desired effect is achieved. In general, a suitable daily dose of a composition of the invention will be that amount of the substance which is the lowest dose effective to produce a therapeutic effect.

Such an effective dose will generally depend upon the factors described above. Preferably, the effective daily dose of a therapeutic composition may be administered as two, three, four, five, six or more sub-doses administered separately at appropriate intervals throughout the day, optionally, in unit dosage forms.

Preferred therapeutic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally or intraperitoneally) to a subject afflicted with the disorders described herein (e.g., HBV infection). Preferred prophylactic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally or intraperitoneally) to a subject. The dose may also be titrated (e.g., the dose may be escalated gradually until signs of toxicity appear, such as headache, diarrhea, or nausea).

The frequency of treatment may also vary. The subject can be treated one or more times per day (e.g., once, twice, three, four or more times) or every so-many hours (e.g., about every 2, 4, 6, 8, 12, or 24 hours). The composition can be administered 1 or 2 times per 24 hours. The time course of treatment may be of varying duration, e.g., for two, three, four, five, six, seven, eight, nine, ten, or more days, two weeks, 1 month, 2 months, 4 months, 6 months, 8 months, 10 months, or more than one year. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Patient Selection and Monitoring

The methods of the present invention described herein entail administration of a compound or composition described herein or a pharmaceutically acceptable salt thereof to a subject to activate the PRR for IFNs, ISGs and cytokines production or additionally induce the expression of PRRs (e.g., RIG-I, STING etc.). In some embodiments, the subject is suffering from or is diagnosed with a condition, e.g., a proliferative disease, e.g., cancer. Accordingly, a patient and/or subject can be selected for treatment using a compound or composition described herein or a pharmaceutically acceptable salt thereof by first evaluating the patient and/or subject to determine whether the subject is infected with a proliferative disease, e.g., cancer. A subject can be evaluated as infected with a proliferative disease (e.g., cancer) using methods known in the art. The subject can also be monitored, for example, subsequent to administration of a compound or composition described herein (e.g., a compound or composition described herein or a pharmaceutically acceptable salt thereof.

In some embodiments, the subject is a mammal. In some embodiments, the subject is a human. In some embodiments, the subject is an adult. In some embodiments, the subject has a proliferative disease, e.g., cancer. In some embodiments, the subject has a cancer of the of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body. In some embodiments, the subject has a cancer comprising a solid tumor (e.g., a carcinoma, a sarcoma, or a lymphoma). In some embodiments, the subject has a hepatocellular carcinoma or other cancer of the liver. In some embodiments, the subject has a leukemia or other cancer of the blood. In some embodiments, the subject has a breast cancer, renal cell carcinoma, colon cancer, melanoma, ovarian cancer, head and neck squamous cell carcinoma, pancreatic cancer, prostate cancer, lung cancer, brain cancer, or gastrointestinal stromal cancer. In some embodiments, the subject has cancer cells (e.g., tumor cells) comprising specific cancer-associated antigens that induce a T-cell response.

In some embodiments, the subject is treatment naïve. In some embodiments, the subject has been previously treated for a proliferative disease (e.g., a cancer). In some embodiments, the subject has relapsed.

### Combination Therapies

A compound or composition described herein may be used in combination with other known therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A compound or composition described herein and the at least one additional therapeutic agent can be administered simultaneously, in the same or in separate compositions, or sequentially. For sequential administration, the compound described herein can be administered first, and the additional agent can be administered second, or the order of administration can be reversed.

In some embodiments, the combination of a compound or composition described herein or a pharmaceutically acceptable salt thereof and the additional agent has a synergistic or additive effect. In some embodiments, the term “additive” refers to an outcome wherein when two agents are used in combination, the combination of the agents acts in a manner equal to but not greater than the sum of the individual activity of each agent.

In some embodiments, the terms “synergy” or “synergistic” refer to an outcome wherein when two agents are used in combination, the combination of the agents acts so as to require a lower concentration of each individual agent than the concentration required to be efficacious in the absence of the other agent. In some embodiments, a synergistic effect results in a reduced in a reduced minimum inhibitory concentration of one or both agents, such that the effect is greater than the sum of the effects. A synergistic effect is greater than an additive effect. In some embodiments, the agents in the composition herein may exhibit a synergistic effect, wherein the activity at a particular concentration is greater than at least about 1.25, 1.5, 1.75, 2, 2.5, 3, 4, 5, 10, 12, 15, 20, 25, 50, or 100 times the activity of either agent alone.

For example, any of the methods described herein may further comprise the administration of a therapeutically effective amount of an additional agent. Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In an embodiment, the additional agent is an immuno oncology agent, for example, an agent that activate the immune system, e.g., making it able to recognize cancer cells and destroy them. Exemplary immono oncology compounds are compounds that inhibit the immune checkpoint blockade pathway. In an embodiment, the compound is an antibody such as a PD-1 or PD-L1 antibody or a co-stimulatory antibody. In another embodiment, the agent is a cell based agent such as CAR-t therapy.

### EXAMPLES

### Example 1. Synthesis of Cmds 1, 7, 8, 13, 14, 15, and 16

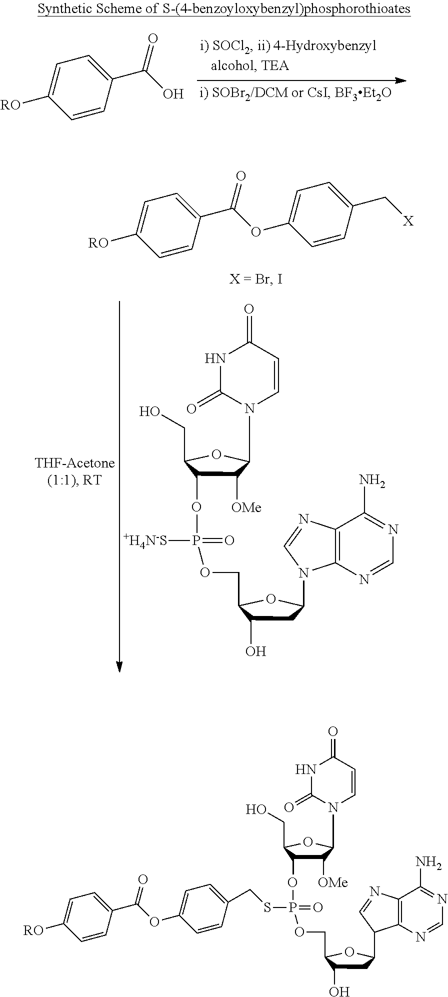

### Typical Synthesis of S-(4-benzoyloxybenzyl)-phosphorothioate Derivative (Cmd 1)

### Step 1: Preparation of 4-Decyloxybenzoyl Chloride

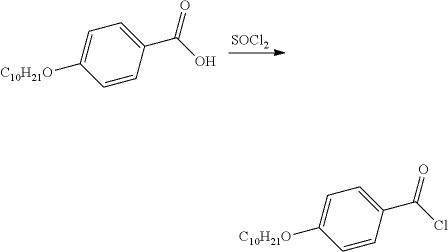

Thionyl chloride (15 mL) was chilled in an ice bath and to this 4-(decyloxy)-benzoic acid (5.0 g, 17.96 mmol) was added. The reaction mixture was stirred at room temperature overnight and next day, concentrated to remove excess thionyl chloride and the crude (1) obtained was used for the next step.

### Step 2: Preparation of 4-Benzoyloxybenzyl Alcohol Derivative

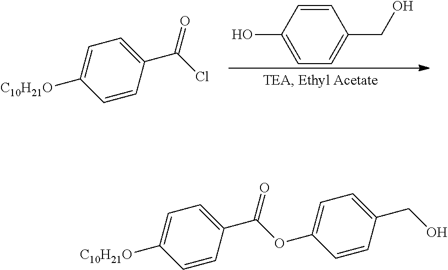

To a suspension of 4-hydroxy benzyl alcohol (2.23 g, 17.96 mmol) in ethyl acetate, chilled in an ice bath, the crude acid chloride (from Step 1, 5.3 g, 17.96 mmol) in ethyl acetate was added followed by the addition of triethylamine (2.0 g, 19.76 mmol). The reaction was monitored by TLC (7:3 Hexanes:Ethyl Acetate) and stopped once the presence of starting material was not detected. LCMS was used to confirm the correct product formation. The reaction was filtered and the precipitate washed with ethyl acetate. Concentrated to give crude product and this was purified by silica gel column in ethyl acetate and heptanes to give the 4-benzoyloxyenzylalcohol (3.4 g).

### Step 3: Preparation of 4-benzoyloxybenzyl Iodide

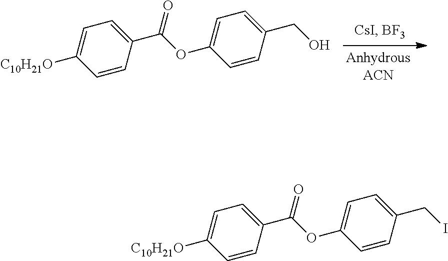

To 4-benzoyloxybenzyl alcohol derivative (from Step 2, 3.4 g, 8.83 mmol) was stirred in anhydrous acetonitrile (85 mL). The compound did not go completely into solution and cesium iodide (3.0 g, 11.48 mmol) and boron trifluoride diethyl etherate (1.63 g, 11.48 mmol) in anhydrous acetonitrile were added to this slurry. The reaction was stirred at room temperature overnight and monitored by TLC (7:3 Hexanes:Ethyl Acetate). As reaction progressed, reaction mixture became a yellow solution. In TLC, the new product spot appeared near the solvent front and, once the starting material was consumed, the reaction mixture was quenched with water. The product was extracted in ethyl acetate and the organic layer was washed with saturated sodium bicarbonate and sodium bisulfite solution. This was then dried over sodium sulfate, filtered, and concentrated to give a crude yield of 3.7 g of iodo product.

### Step 4: Preparation of S-Alkylated Nucleotide Derivative

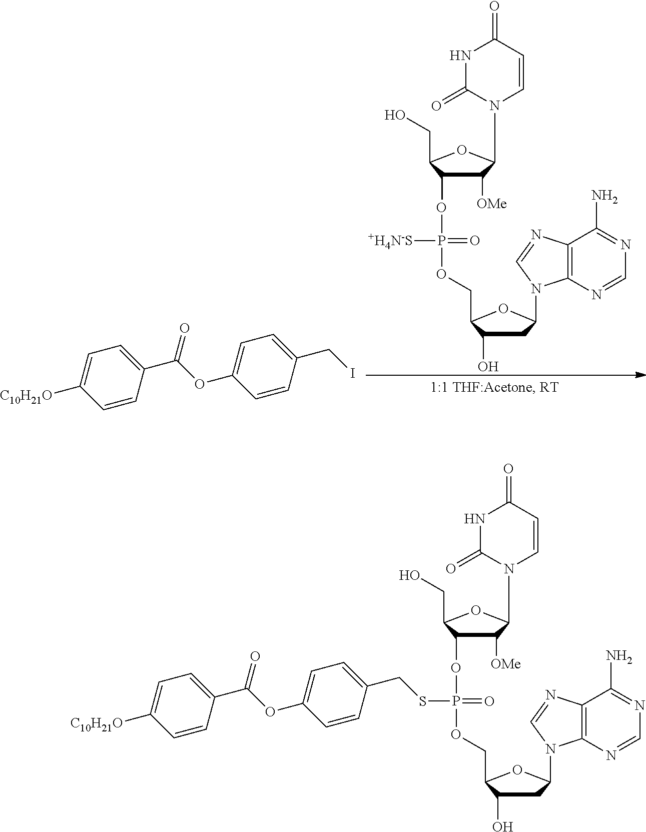

Benzyl iodide derivative (from Step 3, 0.545 g, 1.102 mmol) was dissolved in 1:1 THF:Acetone (6 mL) and the solution was added to the aq. dinucleotide, ApsU_{2′-oMe} solution (1.0 g, 1.653 mmol). The reaction solution became cloudy so additional THF:Acetone (1:1, 2 mL) was added to get a homogenous solution. The reaction was stirred at room temperature overnight and was monitored by TLC (95:5 DCM:Methanol). Following the completion of the reaction, the reaction was worked up. The resulting crude compound was purified on silica gel column using CombiFlash with dichloromethane and isopropanol 0-50%. The appropriate fractions were collected and pure fractions were combined, concentrated, dried to give 1 which was characterized by LCMS, HPLC, and ¹H and ³¹P NMR.

Various compounds, synthesized following the above general procedure, were characterized by HPLC (% purity), LC-MS, and ³¹P-NMR as shown in the Table below:

| Cmd # | LCMS (+mode) | 31P NMR (δ ppm) |
|---|---|---|
| 13 | 875.80 | 29.03, 28.23 |
| 14 | 925.95 | 29.34, 28.56 |
| 15 | 968.12 | 27.25, 26.46 |
| 16 | 898.02 | 27.46, 26.63 |
| 1 | 953.87 | 27.46, 26.64 |
| 7 | 953.93 | 27.33 |
| 8 | 954.06 | 26.54 |

### Example 2. Synthesis of S-alkylnucleoside Phosphorothioate Derivatives

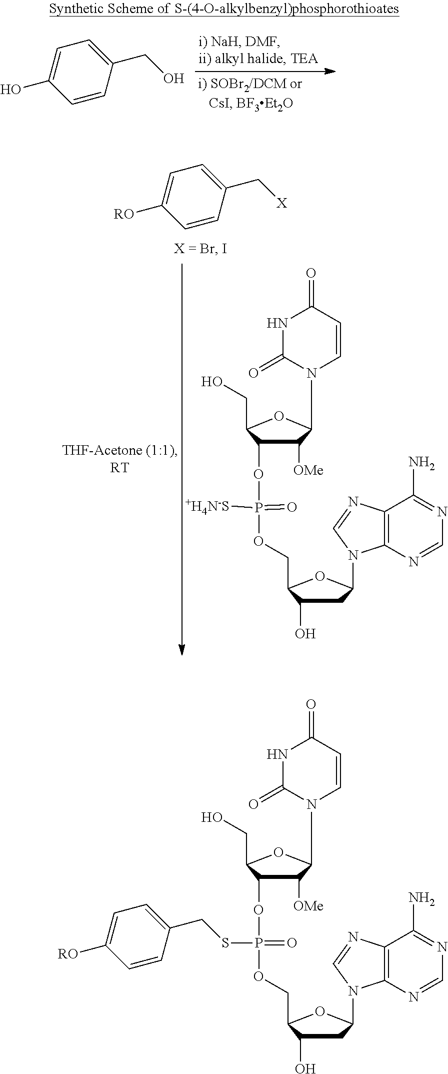

### Step 1: Preparation of 4 (dodecyloxy)benzyl Alcohol

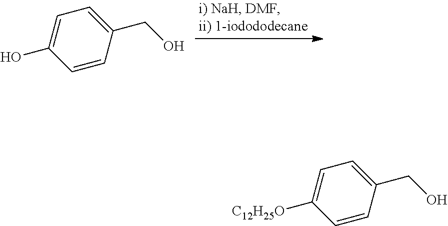

To 4-hydroxybenzyl alcohol (0.62 g, 5 mmol) in anhydrous DMF (7 mL) in ice-water bath NaH (60% suspension, 0.26 g, 1.3 eq) was added and stirred as such for 30 mins under argon. Iodo compound (1.4 mL, 1.1 eq) was added as neat liquid and stirred under argon. As the reaction mixture became additional anhydrous DMF (5 mL) was added and stirred overnight. The reaction mixture was poured into ice cold water, extracted in ether (50 mL), washed with water (10 mL) and later brine (10 mL). Organic layer was dried over anhydrous Na₂SO₄ and organic layer was concentrated under rotavap conditions and later dried high vacuum. 1H-NMR (CDCl₃) 7.26 (d, 2H), 6.88 (d, 2H), 4.61 (d, 2H), 3.95 (t, 2H), 1.78 (t, 2H), 1.45-1.27 (m, 19H), 0.89 (t, 3H) of the isolated product looked good as expected. This was used as such without further purification.

### Step 2: Preparation of 4-(dodecyloxy)benzyl Iodide

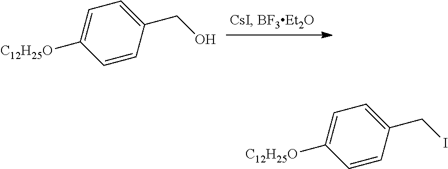

To a suspension of 4-dodecyloxybenzyl alcohol (from step 1, 0.3 g, 1.02 mmol) in anhydrous acetonitrile cesium iodide (0.21 g, 1.13 mmol) was added followed by the addition of boron trifluoride (0.15 mL, 1.1 eq). The dark reaction mixture was stirred under argon overnight, covered with aluminum foil. The reaction mixture was poured into ice cold water (50 mL), extracted in DCM (2×20 mL) and combined organic layer was washed with NaHSO₃ (5%, 10 mL) followed by bring (10 mL) and dried over anhydrous Na₂SO₄. Crude product, obtained after the removal of solvent, was purified by column chromatography on silica using CombiFlash using hexanes and ethyl acetate. Pure fractions were combined, concentrated and dried.

### Step 3: Preparation of S-4-(dodecyloxy)benzyl)nucleotide Derivative

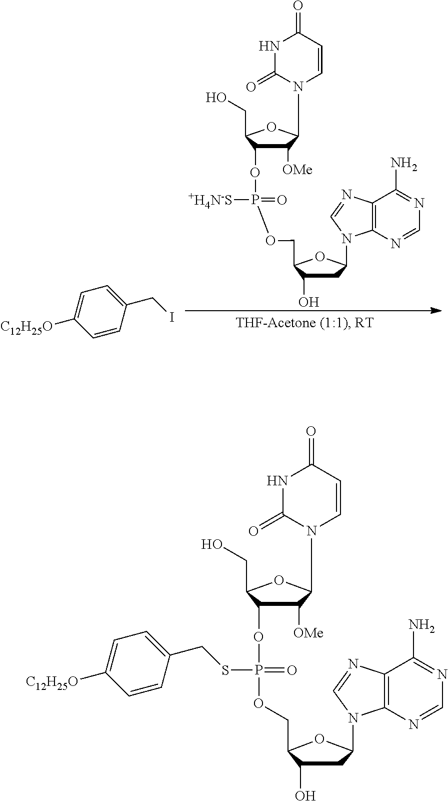

Benzyl iodide derivative (from Step 3, 70 mg, 0.174 mmol) was dissolved in 1:1 THF:Acetone (3 mL) and the solution was added to the aq. dinucleotide, ApsU_{2′-OMe} solution (76 mg, 125 mmol). The reaction mixture was stirred in dark for 96 h, as the reaction remained incomplete and this was concentrated with added silica gel under rotavap conditions. This was used for purification on silica gel column using CombiFlash with DCM and isopropanol 0-50%. The appropriate fractions were collected and purest fractions were combined, concentrated, dried. The isolated product was analyzed by HPLC (93.1% pure), LCMS, 862 (M+1, expected 862.35 for C₃₉H₅₆N₇O₁₁PS) and ³¹P NMR (CD3CN-D₂O) δ 27.9 and 27.1 ppm.

### Example 3. Procedure for the Synthesis of Cmds 2-6

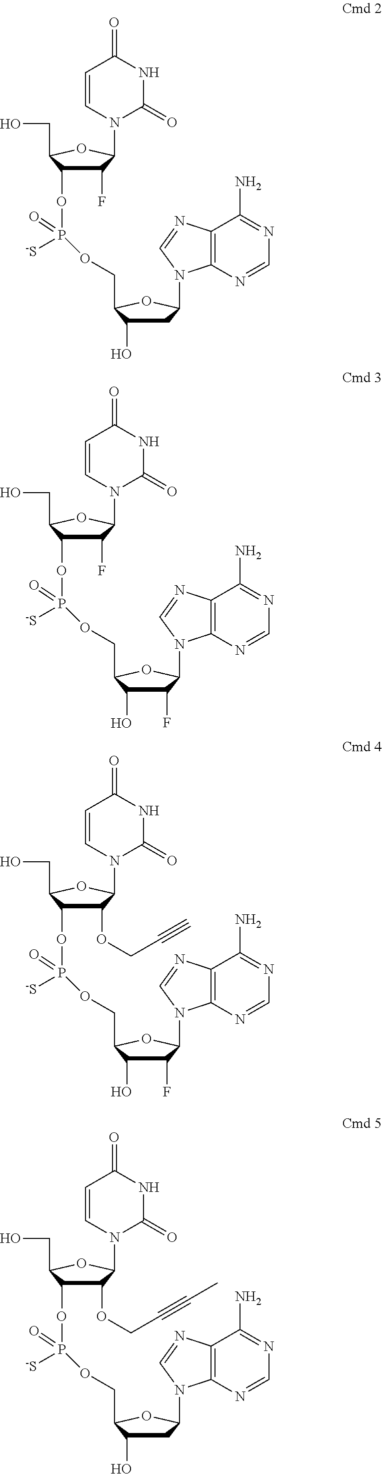

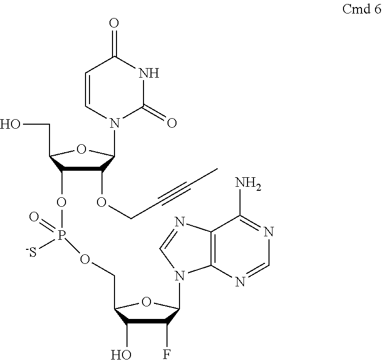

The Cmds 2 and 3 were prepared by solid-phase synthesis using Expedite 8909 DNA Synthesizer at 2 umol synthesis scale. After the synthesis, controlled pore glass (CPG) support was dried and deprotected using aq. NH₃ (400 uL) at room temperature overnight. After deprotection, CPG was filtered off and washed with 3×200 ul HPLC water. Supernatants were combined and concentrated using a speed vac to remove ammonia, following which, the residue was dissolved in 0.5 M NH₄OAc and desalted over Sep-Pak C₁₈ cartridge (Wat 023635 or WAT 020515, WATERS) following the protocol below.

### Desalting Protocol:

1. Sep-Pak C₁₈ cartridge was equilibrated with 10 mL MeCN/water (1:1) followed by 3×10 mL of HPLC water and finally with 10 mL 0.2 M NH₄OAc buffer.
2. The oligonucleotide solution was diluted with 0.2 M NH₄OAc to 10 mL, and the diluted solution was loaded slowly onto the Sep-pak cartridge.
3. After loading, the cartridge was washed with 10 mL 0.1 M NH₄OAc, followed by 10 mL water.
4. The sample was eluted with 90% MeCN/H₂O.
5. The eluted samples were monitored by UV at 260 nm. The fractions with dinucleotides were combined and concentrated by vacuum centrifugation using a speed-vac to remove MeCN and then lyophilized to afford salt-free oligonucleotide solution.

### Example 4. Procedure for the Synthesis of Cmds 4-6

Cmds 4-6 were prepared by manual coupling protocol following standard phosphoramidite chemistry using syringe at 10 uml scale. CPG (120 mg, depending on the loading) corresponding to 10 umol synthesis was placed in an empty twist style synthesis column.
1. Detritylation: To one end of the synthesis column, 12 mL empty syringe was attached, and to the other end a syringe filled with 3-4 mL of 3% dichloroacetic (DCA) acid in anhydrous dichloromethane (DCM) was attached for detritylation. The reagent was pushed back and forth for 5 min. After that, the reagent was taken out, and the CPG was washed with anhydrous DCM and dried under the flow of argon. Detritylation was carried out once more to ensure complete detritylation. The reagent was taken out and the CPG was washed with anhydrous DCM, followed by anhydrous acetonitrile (MeCN) and dried.
2. Coupling: Phosphoramidites (10 eq. excess) required for dinucleotide Cmds 4-6 were prepared in anhydrous MeCN at 0.12 M (700 uL) in a dried 5-mL pear-shaped flask. To that 500 uL of 0.25 Methylthiotetrazole was added and mixed well. A 10 mL syringe was attached to one end of the synthesis column after detritylation. The mixture of phosphoramidite and coupling reagent was syringed out using a 3 mL syringe under argon and attached at the other end of the synthesis column. The reagent was pushed back and forth for approximately 20 min. After coupling, the reagent was taken out and the CPG was washed twice with anhydrous MeCN (2×10 mL) and dried.
3. Sulfurization: Sulfurization was carried out using 4 mL, 0.5 M solution of 3-(N,N-dimethylaminomethylidine)amino)-3H-1,2,4-dithiazole-5-thione in 3:2 anhydrous pyridine/anhydrous MeCN. After that CPG was washed thoroughly with anhydrous MeCN followed by anhydrous DCM and dried.
4. Detritylation: After coupling and sulfurization, the CPG was again detritylated using 3% DCA/DCM and dried.

After the synthesis, controlled pore glass (CPG) support was dried and deprotected using aq. NH₃ (3 mL) at room temperature overnight. After deprotection, CPG was filtered off and washed with 3×500 uL HPLC water. Supernatants were combined and concentrated using a speed-vac to remove ammonia. Further, the residue was dissolved in 0.5 M NH₄OAc and desalted over Sep-pak C₁₈ cartridge (WAT 020515, WATERS corporation) following the protocol as described for Cmds 2 and 3.

Alternatively, dinucleotide solution after concentrating the ammonia solution was taken up in 2 mL HPLC water and extracted with ethyl acetate (3×1.5 mL) to remove benzamide from the solution. The aqueous layer was analyzed by HPLC and LC-MS and lyophilized to afford dinucleotides.

LC-MS Data

| Entry | Compound numbers | LC-MS ESI− |
|---|---|---|
| 1. | Cmd 2 | 573.64 |
| 2. | Cmd 3 | 591.78 |
| 3. | Cmd 4 | 627.86 |
| 4. | Cmd 5 | 623.83 |
| 5. | Cmd 6 | 641.97 |

### Example 5. Experimental Procedures for Synthesis of Cmds 20, 21, 22 and 23. General Procedure for the Suzuki-Miyaura Coupling

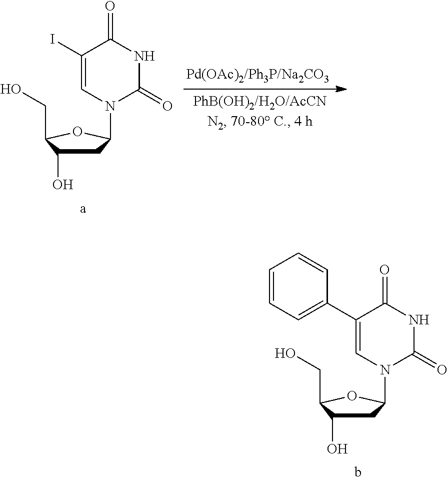 [0465] Reference: Berteina-Raboin, S. et al. Molecules 2012, 17, 14409-14417

To a suspension of 5-iodo-2′-deoxyuridine (a) (5.0 g, 14.12 mmol), phenylboronic acid (2.58 g, 21.18 mmol), sodium carbonate (2.24 g, 21.18 mmol), triphenylphosphine (204 mg, 0.777 mmol) and palladium (II) acetate (124 mg, 0.551 mmol) in water (125 mL) in a 250 mL 1N RB flask was added a stir bar. Acetonitrile (25 mL) was added to give a heterogeneous mixture. Nitrogen was bubbled through this mixture for 3-5 min via a glass pipette followed by the attachment of a 3-way stopcock containing a nitrogen balloon to the neck of the flask. The mixture was heated at 70-80° C. (oil-bath temperature) for 4 h. TLC (DCM/MeOH, 9:1) showed that all the starting material was consumed along with the appearance of a major spot. The reaction mixture which contained some undissolved black/brown particles, was filtered through Celite® and the Celite® was washed with DCM/MeOH (8:2) until TLC of the filtrate indicated no more elution of desired product. The clear filtrate was then evaporated in vacuo and the residue obtained was dried under high vacuum overnight. To this dried residue was added DCM/MeOH (9:1) and the insoluble white solid (Na₂CO₃) was filtered off. Silica gel (20-22 g) was added to the clear filtrate and the solvent was evaporated in vacuo to obtain the crude product as a solid support on silica gel. This crude was then purified by column chromatography (Combiflash, Teledyne Isco) using a gradient of DCM/MeOH to obtain the pure product (4.2 g, 97%) as a white solid that was dried under high vacuum overnight.

### Example 6. General Procedure for Introduction of DMT-Protection on 5′—OH Group

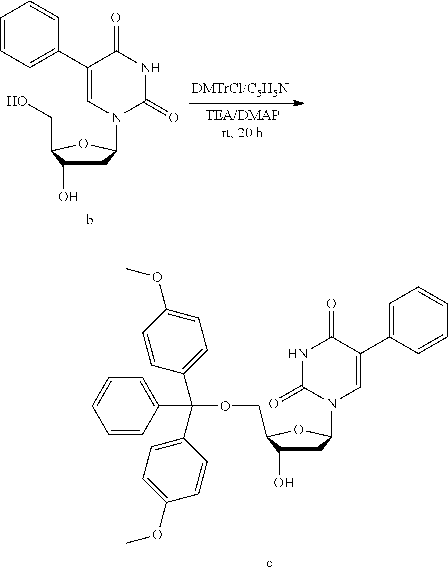

Compound b (4.77 g, 15.68 mmol) was weighed out in a 500 mL 1N RB flask equipped with a stir bar. Dichloromethane (75 mL) and triethylamine (40 mL) was added in which the solid was nearly insoluble. Pyridine (55 mL) was then added and the solid dissolved on stirring to give a clear dark orange solution. This was followed by the addition of DMAP (134 mg, 1.1 mmol) and DMTrCl (6.38 g, 18.82 mmol) in portions at room temperature. The clear orange solution was stirred at room temperature overnight. TLC (DCM/MeOH, 9:1) indicated that all the starting material was consumed. Dichloromethane was then evaporated in vacuo followed by the evaporation of triethylamine/pyridine. The last traces of triethylamine/pyridine were removed by co-evaporation with toluene (2×50 mL). The residue obtained was taken up in ethyl acetate/water and shaken in a separatory funnel. The aqueous layer was discarded and the organic layer was washed with brine. After separating and discarding the brine layer, the organic layer was dried over anhydrous Na₂SO₄, filtered and the solvent evaporated in vacuo to obtain the crude product as a dark brown foamy solid that was dried under high vacuum overnight. The crude material was purified by column chromatography (Combiflash, Teledyne Isco). The silica gel column was first neutralized by equilibrating it with a prepared solution of 5 mL TEA in 1.0 L of DCM. The desired compound 3 was then eluted by running the column with DCM/1.5% TEA in EtOAc. The crude compound was loaded as a liquid in DCM via a disposable syringe. The pure fractions were pooled together and the solvent was evaporated in vacuo to obtain compound c (8.27 g, 87%) as an off-white to pale yellow foam after drying under high vacuum.

### Example 7. General Procedure for Formation of the Nucleoside Phosphoramidite

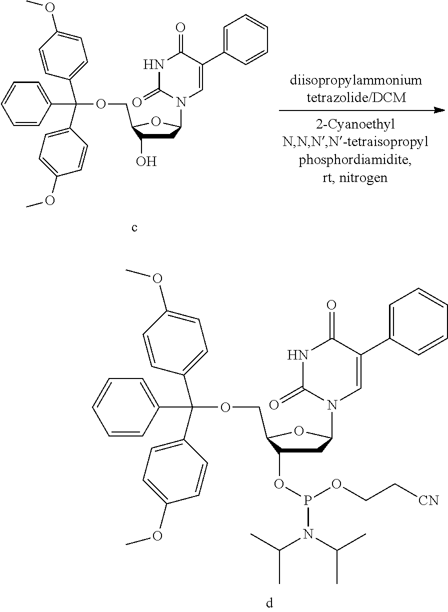

The Compound c (4.8 g, 7.91 mmol) was weighed and transferred to a 500 mL 1N RB flask equipped with a stir bar. Diisopropylammonium tetrazolide (1.35 g, 7.91 mmol) was added and the flask was covered with a septum. A nitrogen-filled balloon was inserted into the septum via a syringe needle and the solids inside the flask were flushed with nitrogen. Anhydrous dichloromethane (150 mL) was added to the flask with stirring to give a clear yellow solution. A solution of 2-cyanoethyl N,N,N′N′-tetraisopropyl phosphoramidite (4.8 g, 15.82 mmol) was prepared in dichlorormethane (20 mL) and this solution was added via a syringe to the clear pale-yellow solution in the flask. After addition, the nearly colorless solution was stirred overnight at room temperature for 18-20 h under nitrogen. After 20 h, TLC (DCM/EtOAc/TEA, 60:40:1) showed the desired product as two non-polar spots for the 2 isomers and no starting material. Deoxygenated dichloromethane (300 mL) was added to the reaction mixture which was then transferred to a separatory funnel, washed with deoxygenated 5% aqueous bicarbonate (200 mL), deoxygenated aqueous 2.5% citric acid (100 mL) and finally deoxygenated water (200 mL). The aqueous layer was discarded and the organic layer was dried over anhydrous Na₂SO₄, filtered and concentrated under reduced pressure to give the desired crude residue (7.25 g) as a yellow foamy solid after drying under high vacuum. ³¹P NMR (CDCl₃): δ 148.430, 148.903. This crude material d was carried over to the next step.

### Example 8. General Procedure for the ETT Coupling and Formation of the Dinucleoside Phosphotriester

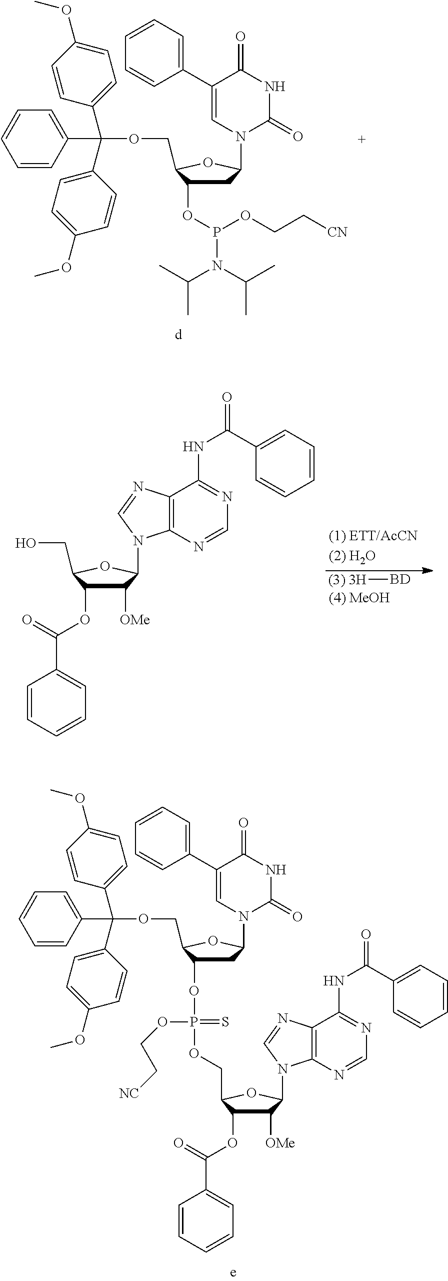

The crude compound d (1.1 g, 1.36 mmol) was weighed and transferred to a 100 mL 1N RB flask equipped with a stir bar and a rubber septum covering its neck. A nitrogen balloon was inserted into the septum via a syringe needle. Anhydrous acetonitrile (25 mL) was then added to the flask and the crude dissolved to give a clear yellow solution. ETT (122 mg, 0.94 mmol) and the dibenzoyl-protected-2′-methoxy adenosine (400 mg, 0.817 mmol) were weighed out in a glass vial and then quickly transferred to the anhydrous solution of the amidite. All the solids dissolved. This solution was stirred at room temperature for 4-5 h under nitrogen. After about 1-2 h into the stirring time, the solution turned slightly cloudy. After 4.5 h, TLC (DCM/MeOH, 98:2, 2× development) showed complete consumption of the dibenzoyl-protected-2′-methoxy adenosine. The reaction mixture was then quenched with water (3.0 μL) and stirred for 5 min after which it turned clear. To this clear crude mixture was added Beaucage-Iyer reagent (3H-BD) (327 mg, 1.634 mmol) as a solid, quickly in a single portion at room temperature and stirred for 45-60 min. After 1 h, TLC (DCM/MeOH, 98:2, 2× development) showed complete consumption of starting material. The reaction mixture was quenched with methanol (2 mL) and the clear solution was stirred for 30 min at room temperature. The solvent was the evaporated in vacuo and the residue was re-dissolved in dichloromethane (150 mL) and washed with water (2×50 mL). The organic layer containing the crude compound e was then dried over anhydrous Na₂SO₄, filtered and kept in the fridge overnight to be carried over to the next step for de-tritylation.

### Example 9. General Procedure for the ETT Coupling and Formation of the Phosphate

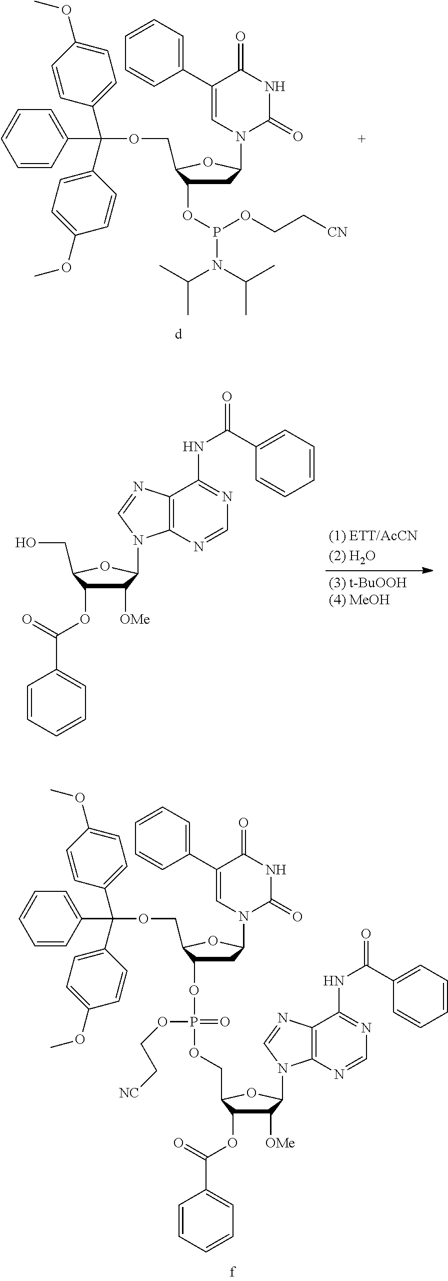

The crude compound e (1.1 g, 1.36 mmol) was weighed and transferred to a 100 mL 1N RB flask equipped with a stir bar and a rubber septum covering its neck. A nitrogen balloon was inserted into the septum via a syringe needle. Anhydrous acetonitrile (25 mL) was then added to the flask and the crude dissolved to give a clear yellow solution. ETT (122 mg, 0.94 mmol) and the dibenzoyl-protected-2′-methoxy adenosine (400 mg, 0.817 mmol) were weighed out in a glass vial and then quickly transferred to the anhydrous solution of the amidite. All the solids dissolved. This solution was stirred at room temperature for 4-5 h under nitrogen. After about 1-2 h into the stirring time, the solution turned slightly cloudy. After 4.5 h, TLC (DCM/MeOH, 98:2, 2× development) showed complete consumption of the dibenzoyl-protected-2′-methoxy adenosine. The reaction mixture was then quenched with water (3.0 μL) and stirred for 5 min after which it turned clear. To this clear crude mixture was added tert-BuOOH (0.45 mL, 2.45 mmol, 5.0-6.0 Min nonane, 3 eq) drop-wise at room temperature. After the addition, the mixture was stirred at room temperature overnight. TLC (DCM/MeOH, 98:2, 2× development) showed complete consumption of starting material. The reaction mixture was quenched with 5% aqueous NaHSO₃ (2 mL) and the solution was stirred for 2 h at room temperature. The solvent was then evaporated in vacuo and the residue was re-dissolved in dichloromethane (150 mL) and washed with water (2×50 mL). The organic layer containing the crude compound f was then dried over anhydrous Na₂SO₄, filtered and kept in the fridge overnight to be carried over to the next step for de-tritylation.

### Example 10. General Procedure for De-Tritylation

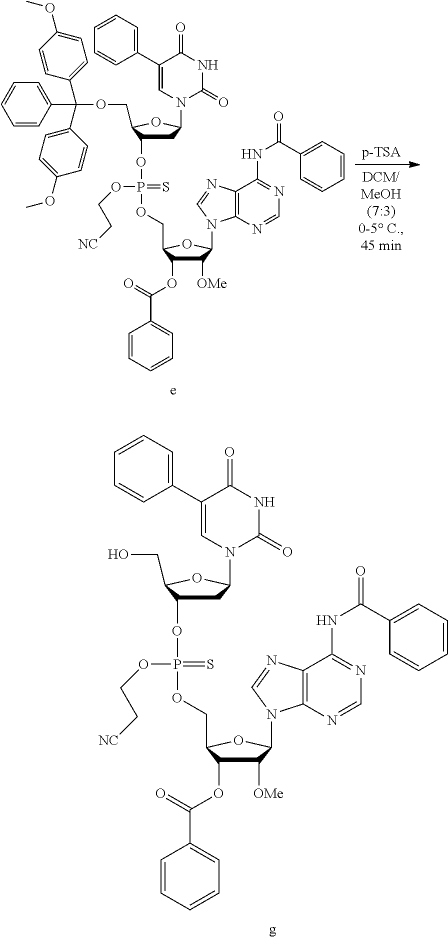

The crude mixture containing compound f (1.0 g, 0.815 mmol) that was dried over anhydrous Na₂SO₄ was filtered and transferred to a 250 mL 1N RB flask equipped with a stir bar. A small portion of the solution was retained as a TLC reference. Dichloromethane was evaporated in vacuo to a pre-marked level of 60 mL in the flask. A thermocouple was immersed into the flask and the flask was cooled to 0-5° C. (internal temperature, ice-water bath). A mixture of p-toluenesulfonic acid (1.5 g, 7.58 mmol) in MeOH/DCM (9 mL/21 mL) was prepared and poured into the 250 mL flask in small portions. The solution immediately turned deep orange and after the addition, this clear deep orange solution was stirred at 0-5° C. for 1 h. The reaction mixture was monitored by TLC (5% MeOH in DCM) and showed complete consumption of the starting material and a major spot. A strong UV active non-polar spot indicated the de-blocked trityl group. Water (50 mL) was added and this biphasic mixture was vigorously stirred for 10 min. During the stirring, the orange color disappeared and an off-white color was observed. The mixture was transferred to a separatory funnel and the lower organic layer was collected in an Erlenmeyer flask. The aqueous layer was re-extracted with dichloromethane (50 mL) and this lower organic layer was combined with the previous one. The combined organic layers were washed with 5% aqueous NaHCO₃ followed by brine, dried over anhydrous Na₂SO₄, filtered and the solvent removed in vacuo to obtain the crude product g as a pale yellow foamy solid that was dried under high vacuum. Yield of crude product was 1.01 g. The crude white foam was dissolved in dichloromethane and directly loaded onto the silica gel column (Combiflash, Teledyne Isco). The crude was purified using a gradient of DCM/MeOH as the eluent to give the desired compound g (457 mg, 60.6%) as a white solid. ³¹P NMR (CDCl₃): δ 66.771; HPLC 97.74%; LCMS 922.77 (−), 924.84 (+).

Similarly, crude compound f was de-tritylated to give compound h (469 mg, 62.5%) as a white solid. ³¹P NMR (CDCl₃): δ −2.567, −2.674; HPLC 97.79%; LCMS 908.97 (−), 907.03 (+).

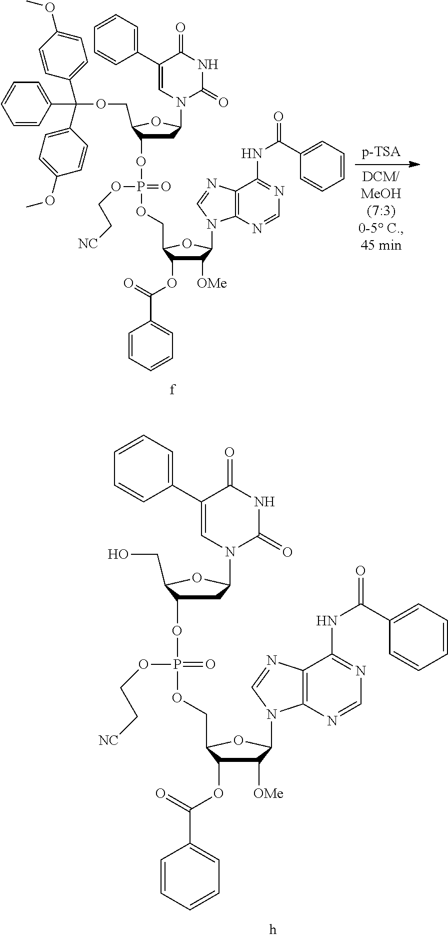

### Example 11. General Procedure for Complete Deprotection

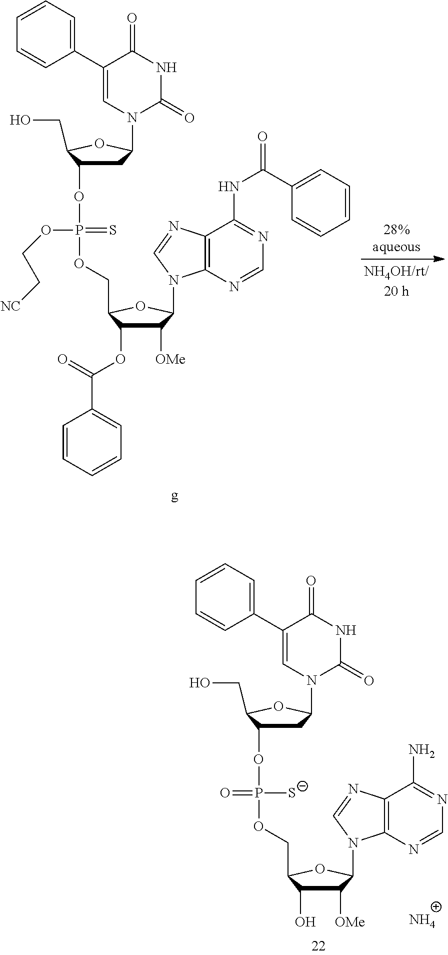

The compound g (525 mg, 0.568 mmol) was transferred to a 250 mL 1N RB flask equipped with a stir bar. To this white solid was added 28% aqueous NH₄OH (40 mL). After about 10 min of stirring time, all the solid dissolved to give a clear, colorless solution. The mixture was stirred at room temperature for 20 h. After 20 h, TLC (20% MeOH in DCM) showed that all the starting material was consumed. Benzamide, formed as the cleavage product was also observed. NH₄OH was then carefully evaporated in vacuo (water-bath=30° C.) and the residue was dissolved in water (130 mL). The water layer was extracted with EtOAc (2×100 mL) to remove benzamide. The organic layers were discarded and the clear, colorless aqueous layer was freeze-dried and lyophilized to obtain compound 22 (364 mg, 94.1%) as a white fluffy solid. ³¹P NMR (DMSO-d₆): δ 54.061, 53.954; HPLC 99.90%; LCMS 661.82 (−), 663.80 (+).

Similarly, compound h was de-protected and the aqueous layer freeze-dried and lyophilized to give compound 20 (385 mg. 92.5%) as a white, fluffy solid. ³¹P NMR (DMSO-d₆): δ-1.712; HPLC 99.5%; LCMS 645.86 (−), 647.84 (+).

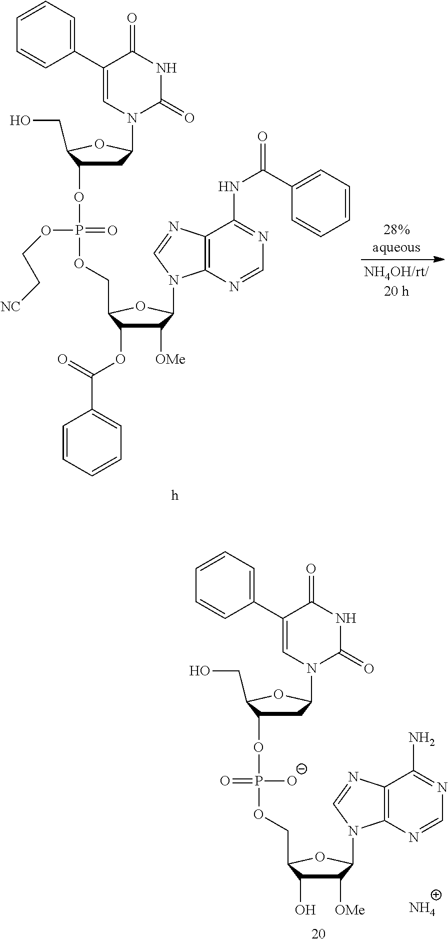

Compound 21 bearing a 2-furyl substituent was synthesized in a similar manner as compound 20. However, the 2-furyl substituent was incorporated via a Stille coupling between 5-iodo-2′-deoxyuridine and 2-(tributylstannyl)furan as shown below (Tor, Y.; Greco, N. J. Tetrahedron 2007, 63, 3515-3527).

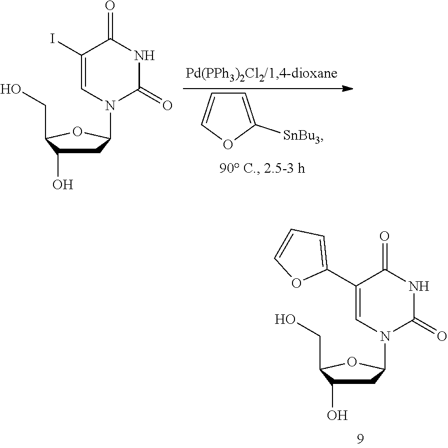

Compound 21: 127.1 mg, 97%; white, fluffy solid; ³¹P NMR (DMSO-d₆): δ −1.689; HPLC 90.0%; LCMS 635.84 (−), 637.81 (+).

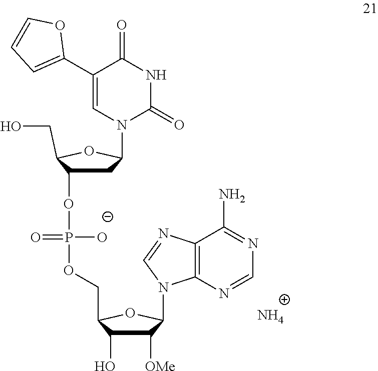

Compound 23 bearing a 2-furyl substituent was synthesized in a similar manner as compound 22. However, the 2-furyl substituent was incorporated via a Stille coupling between 5-iodo-2′-deoxyuridine and 2-(tributylstannyl)furan (Tor, Y.; Greco, N. J. Tetrahedron 2007, 63, 3515-3527).

Compound 23: 193 mg; white, fluffy solid; ³¹P NMR (DMSO-d₆): δ 53.832, 53.725; HPLC 97.44%; LCMS 651.86 (−), 653.77 (+).

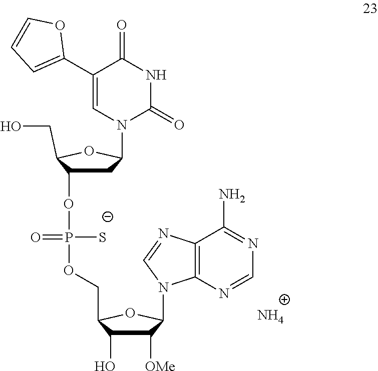

### Example 12. Compounds 1-6 Activate ISG54-Specific SEAP Production in THP1-Blue ISG Cells

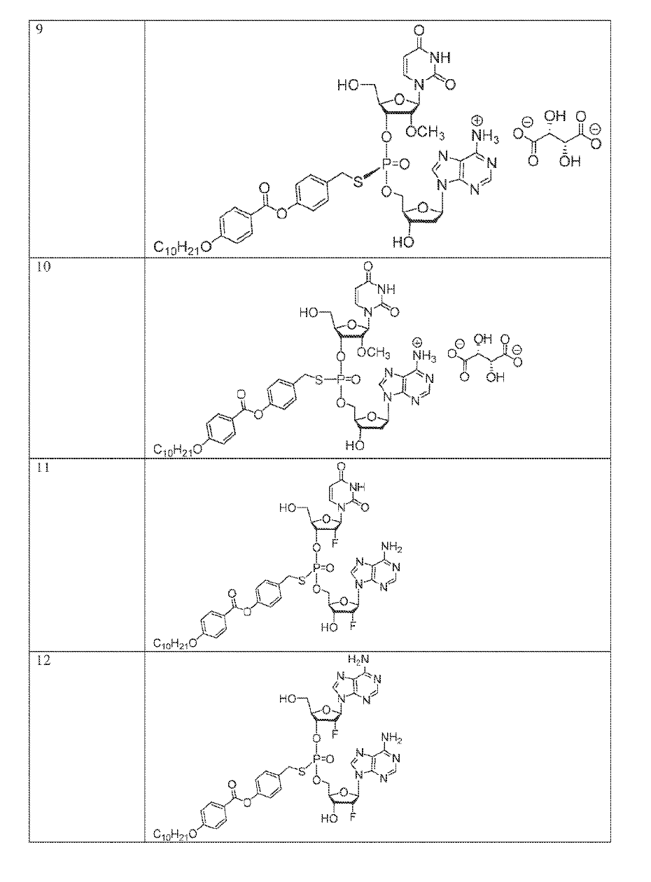

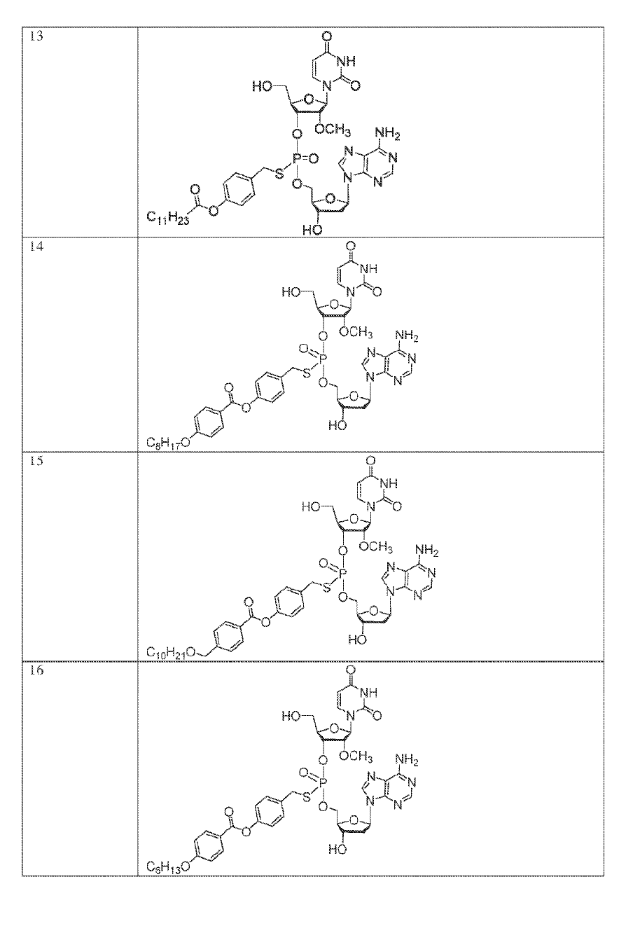

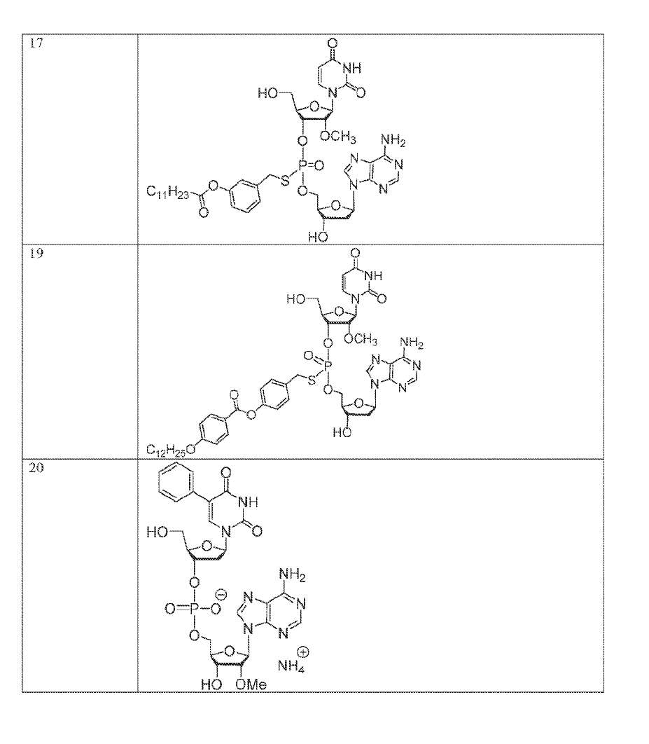

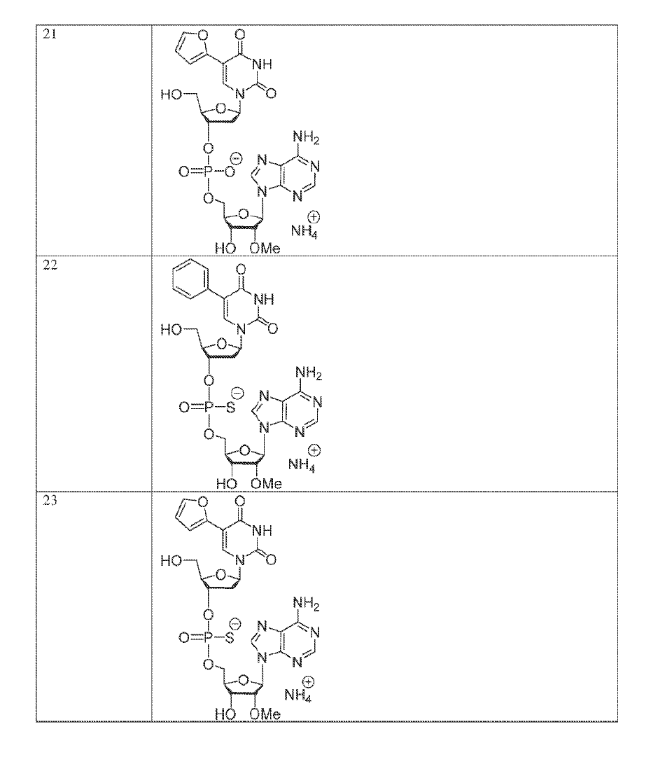

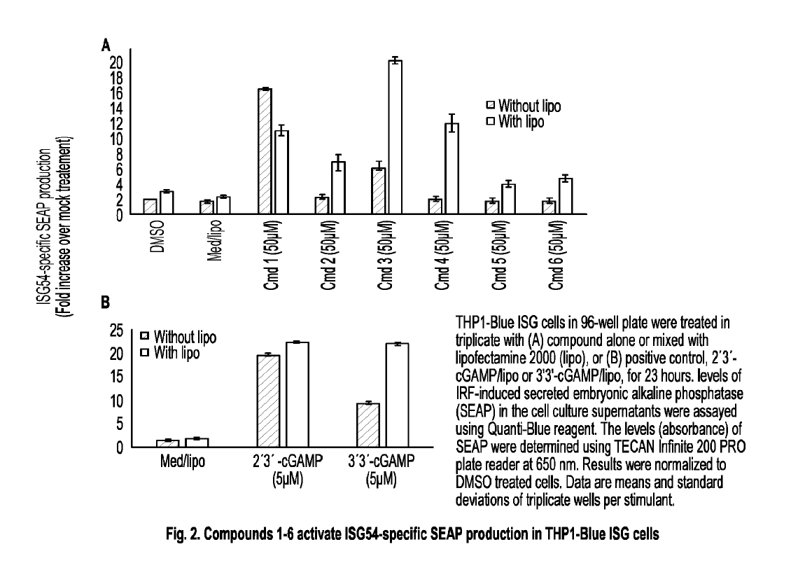

FIG. 2 shows THP1-Blue ISG cells in 96-well plate were treated in triplicate with (A) compound alone or mixed with lipofectamine 2000 (lipo), or (B) positive control, 2′3′-cGAMP/lipo or 3′3′-cGAMP/lipo, for 23 hours. Levels of IRF-induced secreted embryonic alkaline phosphatase (SEAP) in the cell culture supernatants were assayed using Quanti-Blue reagent. The levels (absorbance) of SEAP were determined using TECAN Infinite 200 PRO plate reader at 650 nm. Results were normalized to DMSO treated cells. Data are means and standard deviations of triplicate wells per stimulant.

### Example 13. IRF Induction by Cmd 1 in THP1 Cells

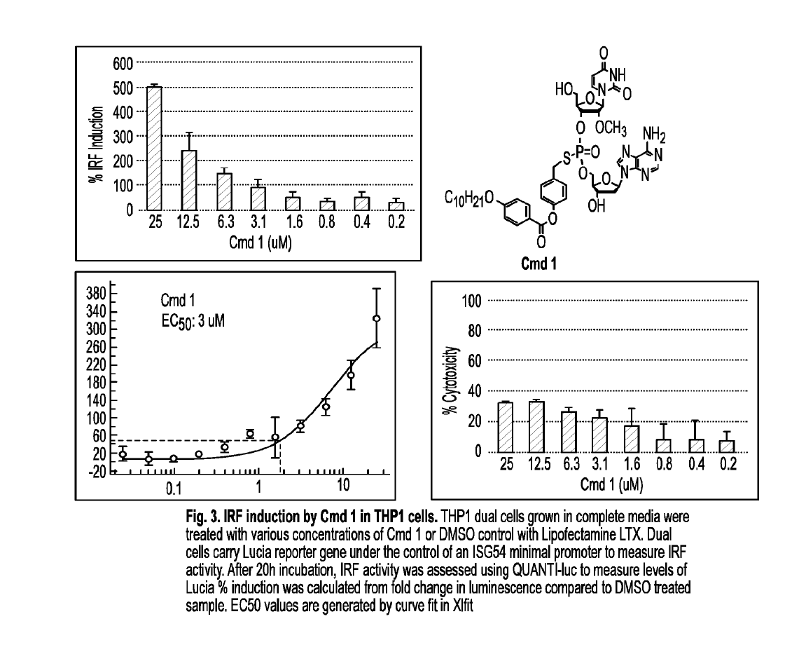

FIG. 3 shows THP1 dual cells grown in complete media were treated with various concentrations of Cmd 1 or DMSO control with Lipofectamine LTX. Dual cells carry Lucia reporter gene under the control of an ISG54 minimal promoter to measure IRF activity. After 20 h incubation, IRF activity was assessed using QUANTI-luc to measure levels of Lucia % induction was calculated from fold change in luminescence compared to DMSO treated sample. EC50 values are generated by curve fit in Xlfit.

### Example 14. Compound 1 Induces a STING-Dependent Type I IFN Response in THP1 Cells in a Dose-Dependent Manner

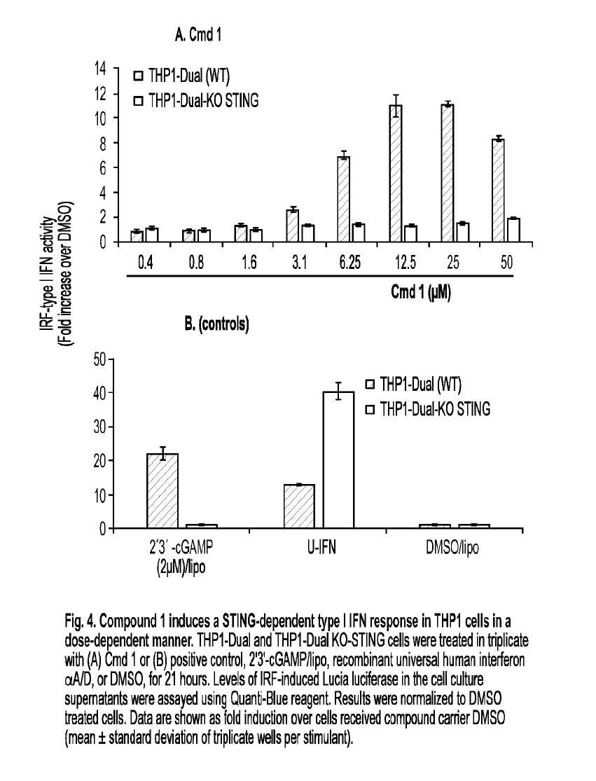

FIG. 4 shows THP1-Dual and THP1-Dual KO-STING cells were treated in triplicate with (A) Cmd 1 or (B) positive control, 2′3′-cGAMP/lipo, recombinant universal human interferon αA/D, or DMSO, for 21 hours. Levels of IRF-induced Lucia luciferase in the cell culture supernatants were assayed using Quanti-Blue reagent. Results were normalized to DMSO treated cells. Data are shown as fold induction over cells received compound carrier DMSO (mean±standard deviation of triplicate wells per stimulant).

### Example 15. IRF Activity of Compounds 3 and 4

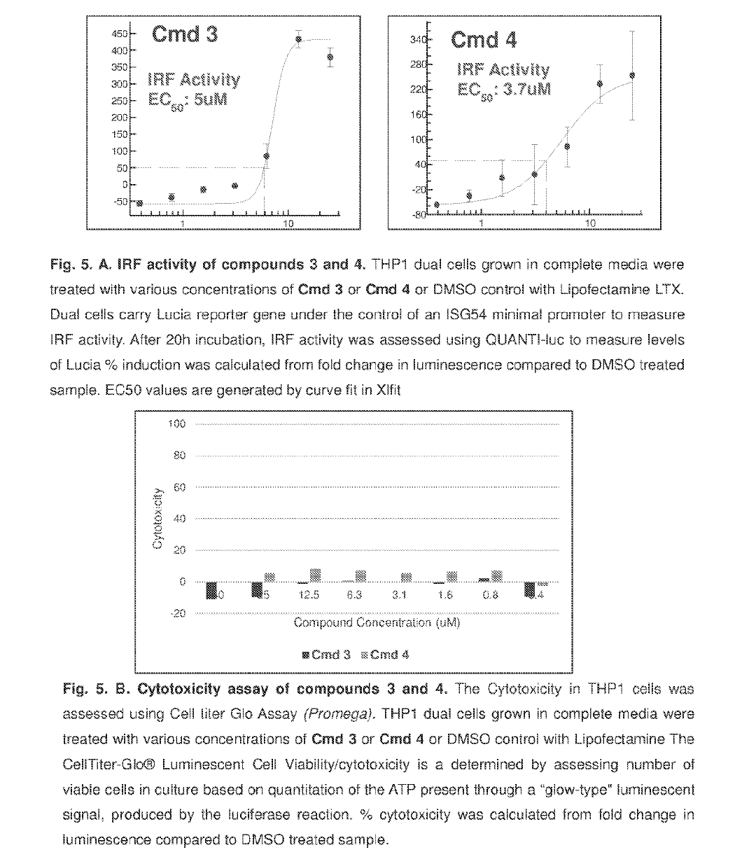

FIG. 5A shows THP1 dual cells grown in complete media were treated with various concentrations of Cmd 3 or Cmd 4 or DMSO control with Lipofectamine LTX. Dual cells carry Lucia reporter gene under the control of an ISG54 minimal promoter to measure IRF activity. After 20 h incubation, IRF activity was assessed using QUANTI-luc to measure levels of Lucia % induction was calculated from fold change in luminescence compared to DMSO treated sample. EC50 values are generated by curve fit in Xlfit.

### Example 16. Cytotoxicity Assay of Compounds 3 and 4

FIG. 5B shows the cytotoxicity in THP1 cells was assessed using Cell titer Glo Assay (Promega). THP1 dual cells grown in complete media were treated with various concentrations of Cmd 3 or Cmd 4 or DMSO control with Lipofectamine. The CellTiter-Glo® Luminescent Cell Viability/cytotoxicity is a determined by assessing number of viable cells in culture based on quantitation of the ATP present through a “glow-type” luminescent signal, produced by the luciferase reaction. % cytotoxicity was calculated from fold change in luminescence compared to DMSO treated sample.

### Example 17. IRF Induction by Compounds 3 and 4 is STING-Dependent

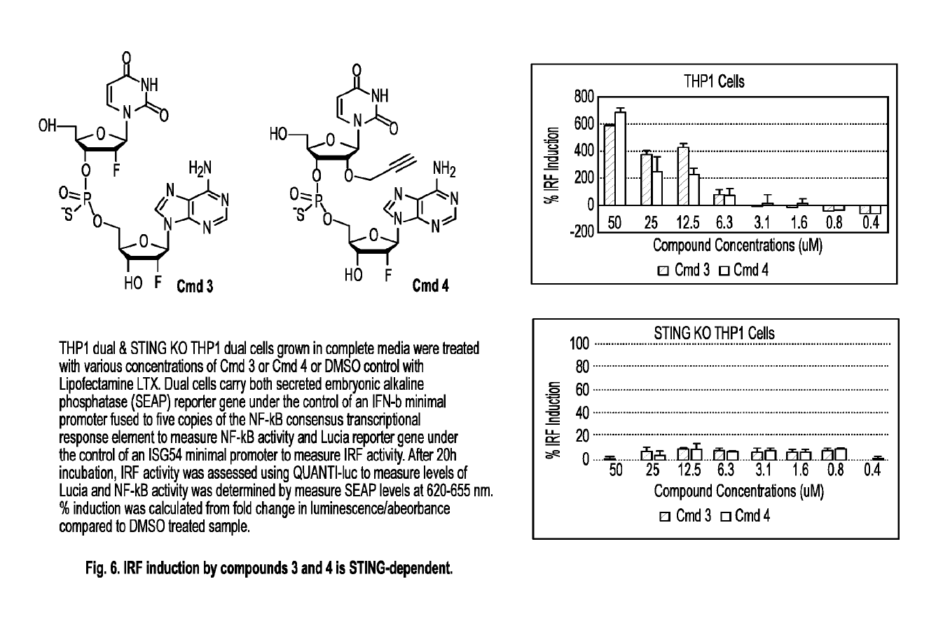

FIG. 6 shows THP1 dual & STING KO THP1 dual cells grown in complete media were treated with various concentrations of Cmd 3 or Cmd 4 or DMSO control with Lipofectamine LTX. Dual cells carry both secreted embryonic alkaline phosphatase (SEAP) reporter gene under the control of an IFN-b minimal promoter fused to five copies of the NF-kB consensus transcriptional response element to measure NF-kB activity and Lucia reporter gene under the control of an ISG54 minimal promoter to measure IRF activity. After 20 h incubation, IRF activity was assessed using QUANTI-luc to measure levels of Lucia and NF-kB activity was determined by measure SEAP levels at 620-655 nm. % induction was calculated from fold change in luminescence/absorbance compared to DMSO treated sample.

### Example 18. STING Pathway Plays a Critical Role in Type I IFN Response Induced by Compounds in THP1 Cells

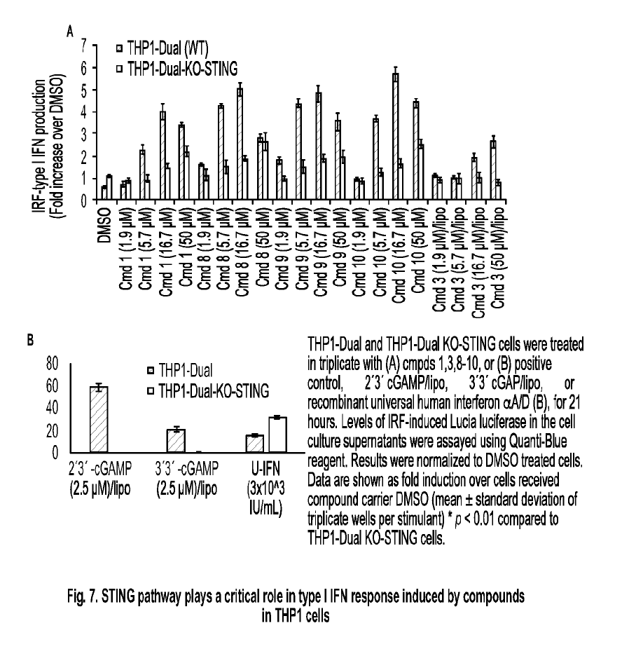

FIG. 7 shows THP1-Dual and THP1-Dual KO-STING cells were treated in triplicate with (A) cmpds 1,3,8-10, or (B) positive control, 2′3′-cGAMP/lipo, 3′3′-cGAMP/lipo, or recombinant universal human interferon αA/D (B), for 21 hours. Levels of IRF-induced Lucia luciferase in the cell culture supernatants were assayed using Quanti-Blue reagent.

Results were normalized to DMSO treated cells. Data are shown as fold induction over cells received compound carrier DMSO (mean±standard deviation of triplicate wells per stimulant). * p<0.01 compared to THP1-Dual KO-STING cells.

### Example 19. IRF Induction by Cmd 7 in THP1 Cells

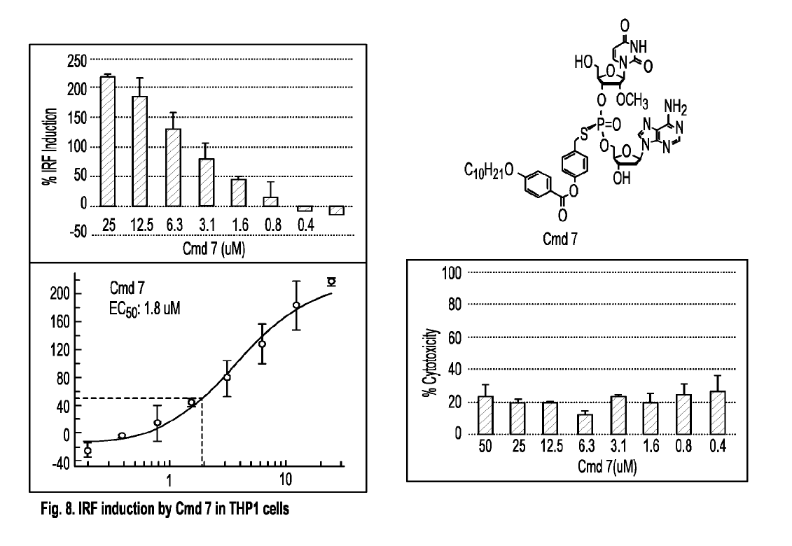

FIG. 8 shows IRF induction by Cmd 7 in THP1 cells.

### Example 20. Compounds 1, 7, and 8 Induces Dose-Dependent ISG54-Specific SEAP Production in THP1-Blue ISG Cells

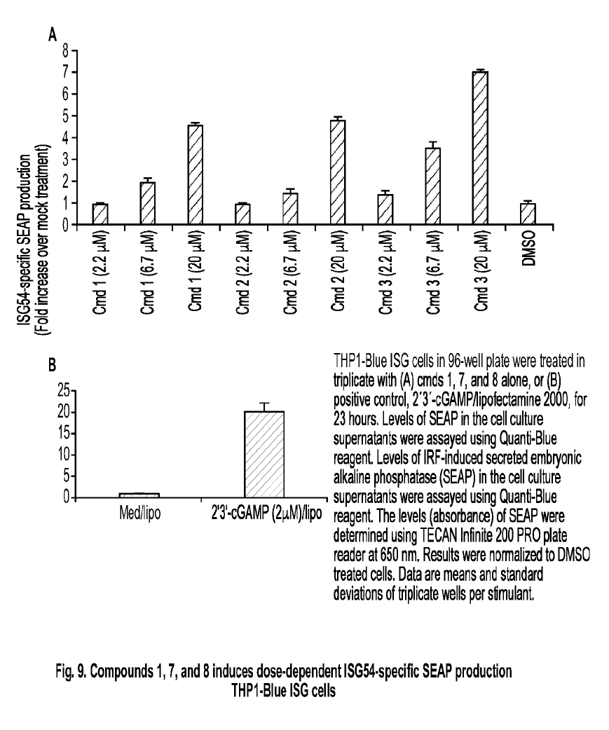

FIG. 9 shows THP1-Blue ISG cells in 96-well plate were treated in triplicate with (A) Cmds 1, 7, and 8 alone, or (B) positive control, 2′ 3′-cGAMP/lipofectamine 2000, for 23 hours. Levels of SEAP in the cell culture supernatants were assayed using Quanti-Blue reagent. Levels of IRF-induced secreted embryonic alkaline phosphatase (SEAP) in the cell culture supernatants were assayed using Quanti-Blue reagent. The levels (absorbance) of SEAP were determined using TECAN Infinite 200 PRO plate reader at 650 nm. Results were normalized to DMSO treated cells. Data are means and standard deviations of triplicate wells per stimulant.

### Example 21. IRF-, and NF-kB-Inducing Activity of Compounds 11 and 12

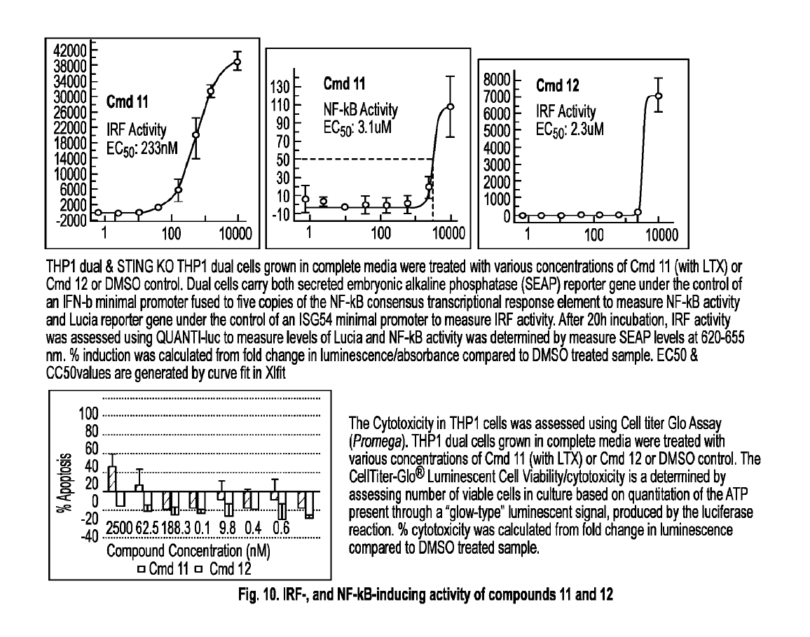

FIG. 10 shows THP1 dual & STING KO THP1 dual cells grown in complete media were treated with various concentrations of Cmd 11 (with LTX) or Cmd 12 or DMSO control.

Dual cells carry both secreted embryonic alkaline phosphatase (SEAP) reporter gene under the control of an IFN-b minimal promoter fused to five copies of the NF-kB consensus transcriptional response element to measure NF-kB activity and Lucia reporter gene under the control of an ISG54 minimal promoter to measure IRF activity. After 20 h incubation, IRF activity was assessed using QUANTI-luc to measure levels of Lucia and NF-kB activity was determined by measure SEAP levels at 620-655 nm. % induction was calculated from fold change in luminescence/absorbance compared to DMSO treated sample. EC50 & CC50 values are generated by curve fit in Xlfit. The Cytotoxicity in THP1 cells was assessed using Cell titer Glo Assay (Promega). THP1 dual cells grown in complete media were treated with various concentrations of Cmd 11 (with LTX) or Cmd 12 or DMSO control. The CellTiter-Glo® Luminescent Cell Viability/cytotoxicity is a determined by assessing number of viable cells in culture based on quantitation of the ATP present through a “glow-type” luminescent signal, produced by the luciferase reaction. % cytotoxicity was calculated from fold change in luminescence compared to DMSO treated sample.

### Example 22. IRF Induction by Compounds 11 & 12 is STING-Dependent

FIG. 11 shows THP1 dual & STING KO THP1 dual cells grown in complete media were treated with various concentrations of Cmd 11 (with LTX) or Cmd 12 or DMSO control. Dual cells carry both secreted embryonic alkaline phosphatase (SEAP) reporter gene under the control of an IFN-b minimal promoter fused to five copies of the NF-kB consensus transcriptional response element to measure NF-kB activity and Lucia reporter gene under the control of an ISG54 minimal promoter to measure IRF activity. After 20 h incubation, IRF activity was assessed using QUANTI-luc to measure levels of Lucia and NF-kB activity was determined by measure SEAP levels at 620-655 nm. % induction was calculated from fold change in luminescence/absorbance compared to DMSO treated sample.

### Example 24. IRF Induction by Cmd 14 in THP1 Cells

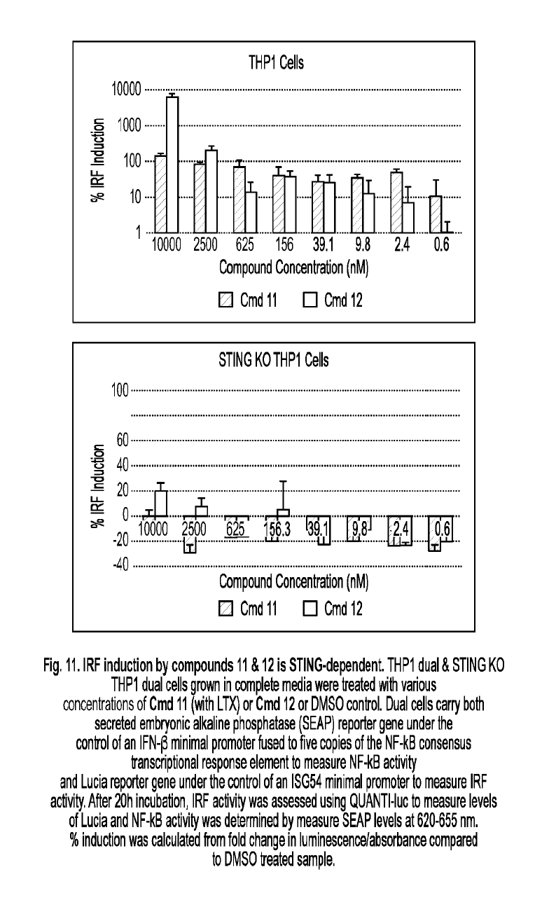

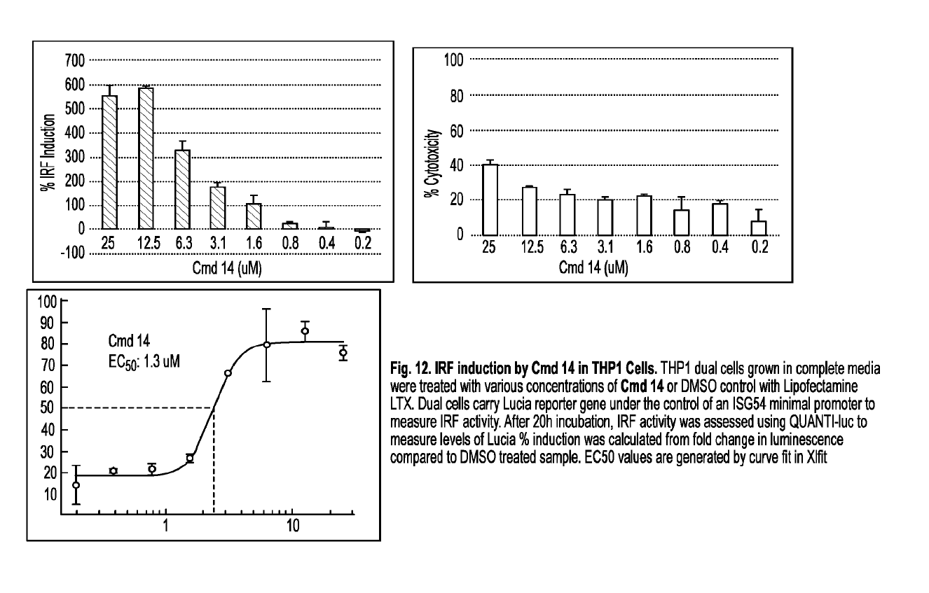

FIG. 12 shows THP1 dual cells grown in complete media were treated with various concentrations of Cmd 14 or DMSO control with Lipofectamine LTX. Dual cells carry Lucia reporter gene under the control of an ISG54 minimal promoter to measure IRF activity. After 20 h incubation, IRF activity was assessed using QUANTI-luc to measure levels of Lucia % induction was calculated from fold change in luminescence compared to DMSO treated sample. EC50 values are generated by curve fit in Xlfit.

### Example 25. IRF Induction by Cmd 15 in THP-1 Cells

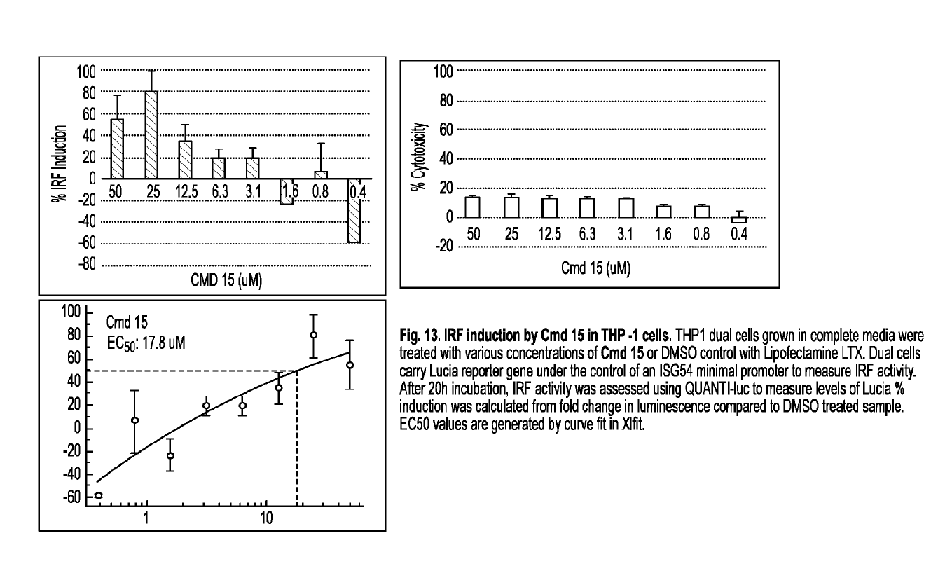

FIG. 13 shows THP1 dual cells grown in complete media were treated with various concentrations of Cmd 15 or DMSO control with Lipofectamine LTX. Dual cells carry Lucia reporter gene under the control of an ISG54 minimal promoter to measure IRF activity. After 20 h incubation, IRF activity was assessed using QUANTI-luc to measure levels of Lucia % induction was calculated from fold change in luminescence compared to DMSO treated sample. EC50 values are generated by curve fit in Xlfit.

### Example 26. IRF Induction by Cmd 16 in THP1 Cells

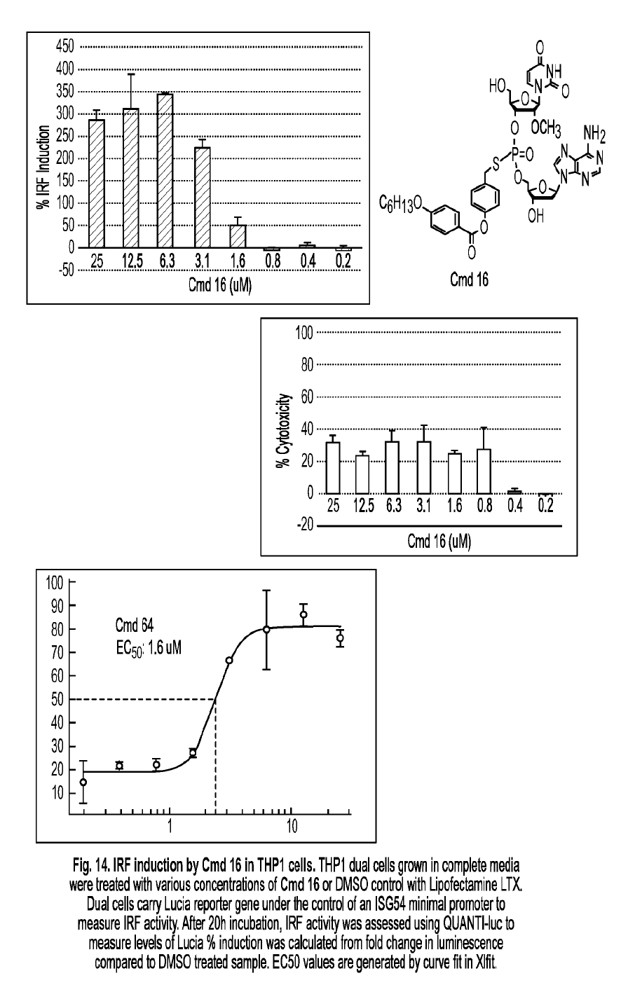

FIG. 14 shows THP1 dual cells grown in complete media were treated with various concentrations of Cmd 16 or DMSO control with Lipofectamine LTX. Dual cells carry Lucia reporter gene under the control of an ISG54 minimal promoter to measure IRF activity. After 20 h incubation, IRF activity was assessed using QUANTI-luc to measure levels of Lucia % induction was calculated from fold change in luminescence compared to DMSO treated sample. EC50 values are generated by curve fit in Xlfit Example 27. IRF Induction by Compounds 20-23.

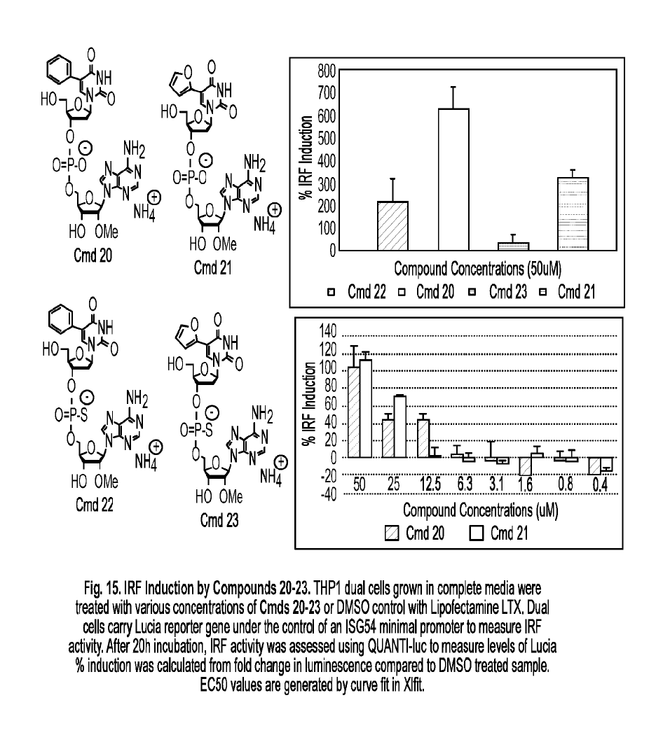

FIG. 15 shows THP1 dual cells grown in complete media were treated with various concentrations of Cmds 20-23 or DMSO control with Lipofectamine LTX. Dual cells carry Lucia reporter gene under the control of an ISG54 minimal promoter to measure IRF activity. After 20 h incubation, IRF activity was assessed using QUANTI-luc to measure levels of Lucia % induction was calculated from fold change in luminescence compared to DMSO treated sample. EC50 values are generated by curve fit in Xlfit.

### Example 28. Compound 16 Induces a STING-Dependent Type I IFN Response in THP1 Cells

FIG. 16 shows THP1-Dual and THP1-Dual-KO STING cells were treated in triplicate with indicated compounds or controls for 21 hrs. Levels of IRF-induced Lucia luciferase in the cell culture supernatants were assayed using Quanti-Blue reagent. Results were normalized to DMSO treated cells. Data are shown as fold induction over cells received compound carrier DMSO (mean±standard deviation of triplicate wells per stimulant).

### Example 29. Compound 1 Induces the Expression of IFN-β and IRF7 in THP1 Cells

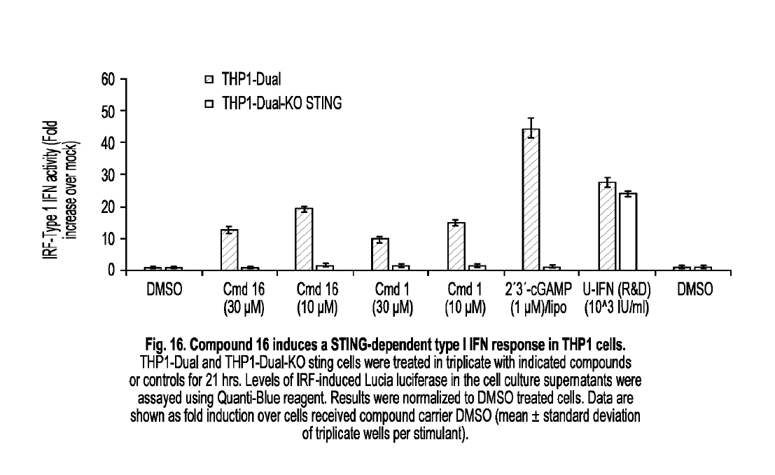

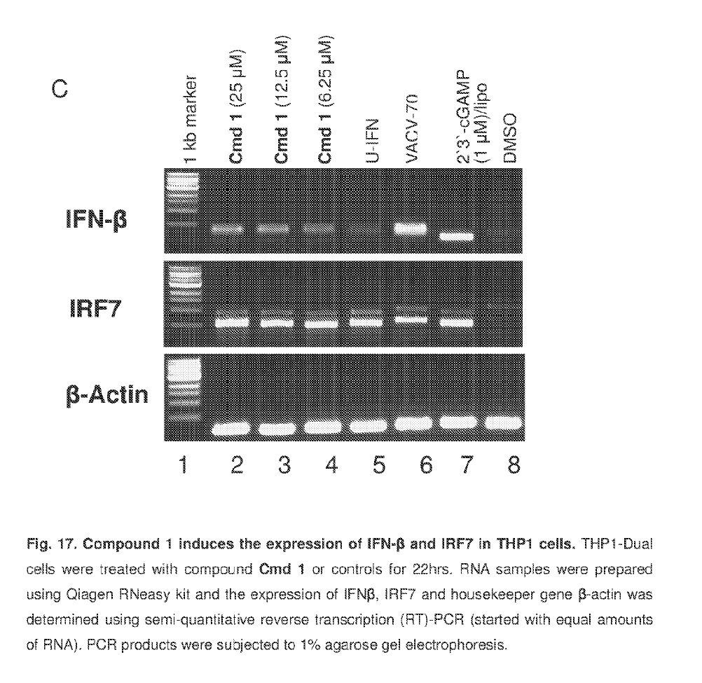

FIG. 17 shows THP1-Dual cells were treated with compound Cmd 1 or controls for 22 hrs. RNA samples were prepared using Qiagen RNeasy kit and the expression of IFNβ, IRF7 and housekeeper gene β-actin was determined using semi-quantitative reverse transcription (RT)-PCR (started with equal amounts of RNA). PCR products were subjected to 1% agarose gel electrophoresis.

### Example 30. 2′3′-cGAMP Induces IFN-β Gene Expression within 5 Hrs; it Takes >5 Hrs for Compound 1 to Activate IFN-β Gene Expression in THP1-WT

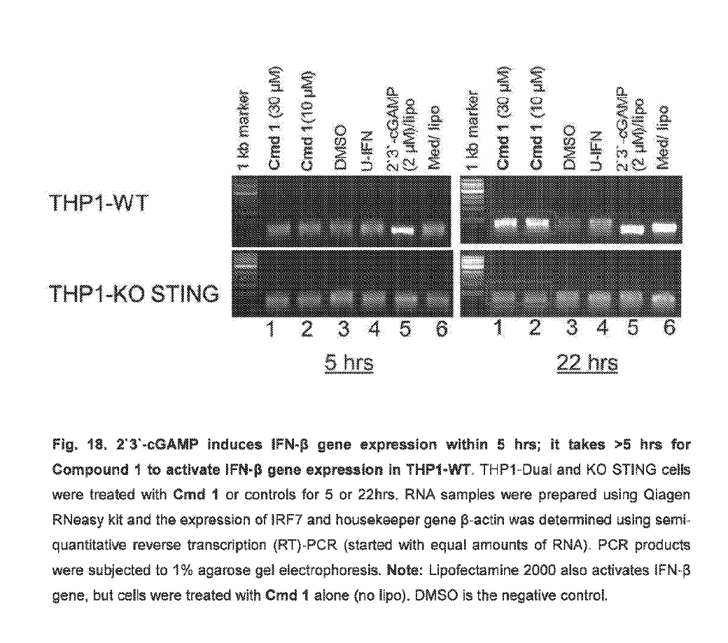

FIG. 18 shows THP1-Dual and KO STING cells were treated with Cmd 1 or controls for 5 or 22 hrs. RNA samples were prepared using Qiagen RNeasy kit and the expression of IRF7 and housekeeper gene 3-actin was determined using semi-quantitative reverse transcription (RT)-PCR (started with equal amounts of RNA). PCR products were subjected to 1% agarose gel electrophoresis. Lipofectamine 2000 also activates IFN-β gene, but cells were treated with Cmd 1 alone (no lipo). DMSO is the negative control.

### Example 31. Compound 7 Induces the Expression of IFN-β and IRF7 in THP1 Cells in STING-Dependent Manner

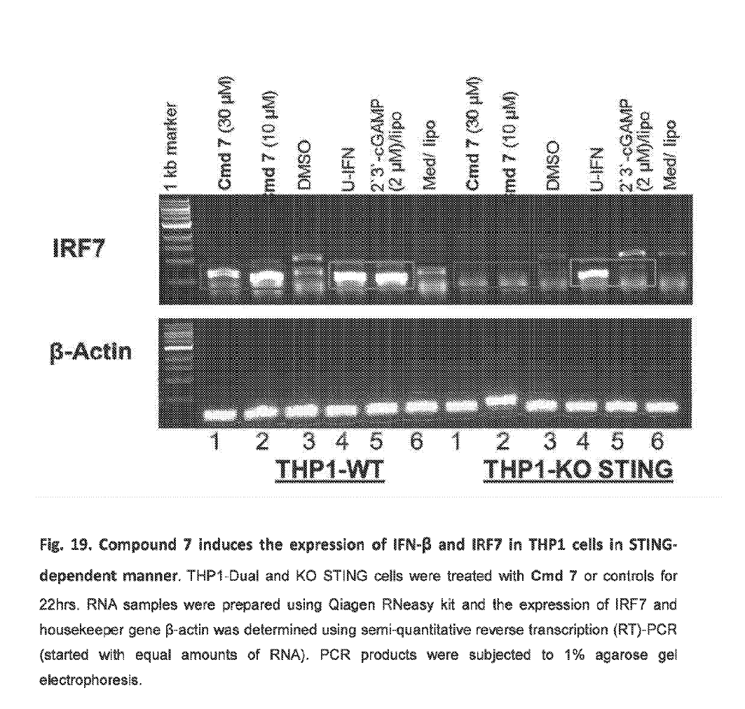

FIG. 19 shows THP1-Dual and KO STING cells were treated with Cmd 7 or controls for 22 hrs. RNA samples were prepared using Qiagen RNeasy kit and the expression of IRF7 and housekeeper gene β-actin was determined using semi-quantitative reverse transcription (RT)-PCR (started with equal amounts of RNA). PCR products were subjected to 1% agarose gel electrophoresis.

### Example 32. The cGAS Pathway Appears Important for Compounds 1 and 7 Induced Type I IFN Responses

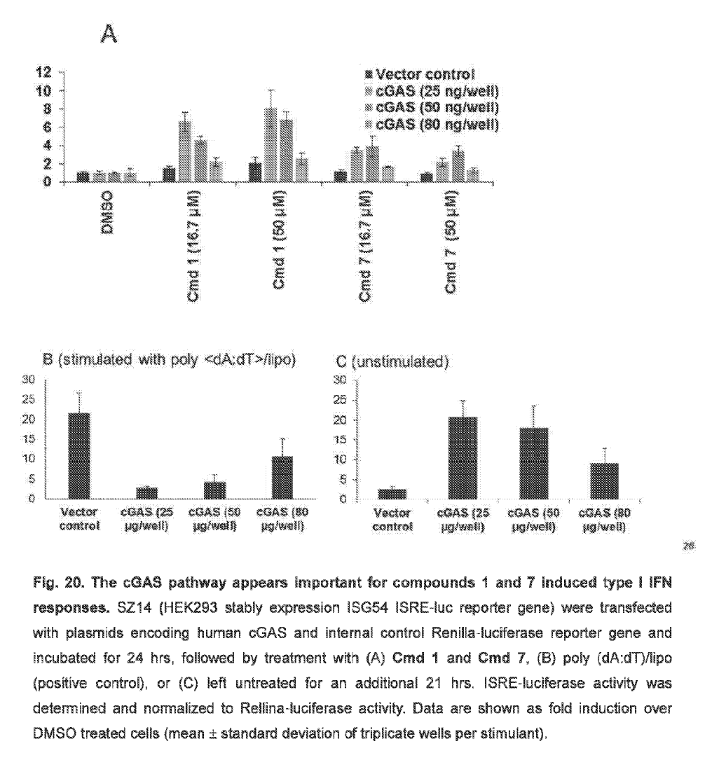

FIG. 20 shows SZ14 (HEK293 stably expression ISG54 ISRE-luc reporter gene) were transfected with plasmids encoding human cGAS and internal control Renilla-luciferase reporter gene and incubated for 24 hrs, followed by treatment with (A) Cmd 1 and Cmd 7, (B) poly (dA:dT)/lipo (positive control), or (C) left untreated for an additional 21 hrs. ISRE-luciferase activity was determined and normalized to Rellina-luciferase activity. Data are shown as fold induction over DMSO treated cells (mean±standard deviation of triplicate wells per stimulant).

### Example 33. K384 and K411 Residues in cGAS Appear Important in Mediating Compound 1 Activation of STING-Dependent Type I IFN Signaling

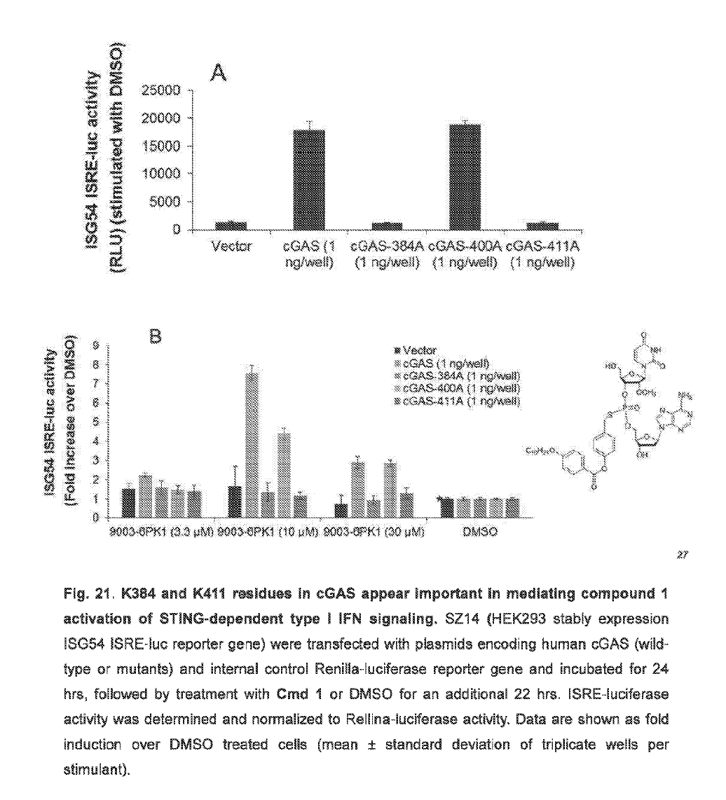

FIG. 21 shows SZ14 (HEK293 stably expression ISG54 ISRE-luc reporter gene) were transfected with plasmids encoding human cGAS (wild-type or mutants) and internal control Renilla-luciferase reporter gene and incubated for 24 hrs, followed by treatment with Cmd 1 or DMSO for an additional 22 hrs. ISRE-luciferase activity was determined and normalized to Rellina-luciferase activity. Data are shown as fold induction over DMSO treated cells (mean±standard deviation of triplicate wells per stimulant).

### Example 34. RIG-I, MDA5, LGP2, OAS1 and ISG54 Gene Expression in THP1 after Cmd 1, Poly IC & dsRNA Treatment

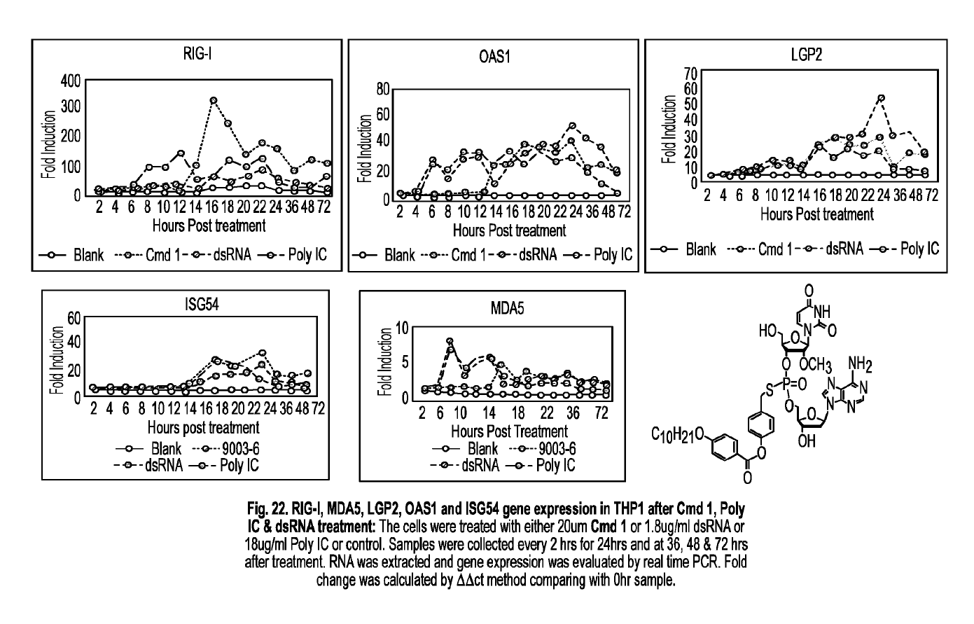

FIG. 22 shows the cells were treated with either 20 uM Cmd 1 or 1.8 ug/mL dsRNA or 18 ug/mL Poly IC or control. Samples were collected every 2 hrs for 24 hrs and at 36, 48 & 72 hrs after treatment. RNA was extracted and gene expression was evaluated by real time PCR. Fold change was calculated by ΔΔct method comparing with 0 hr sample.

### Example 35. Dose Dependent Induction of Various ISGs in THP1 Cells by Cmd 7. Gene Expression Analysis in THP1 after Cmd 7 Treatment

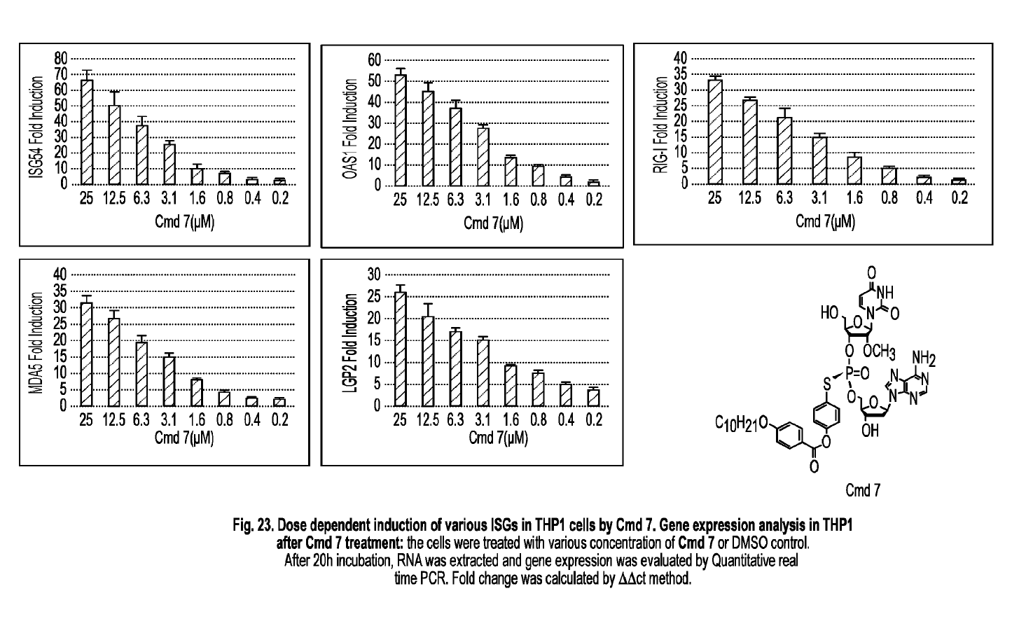

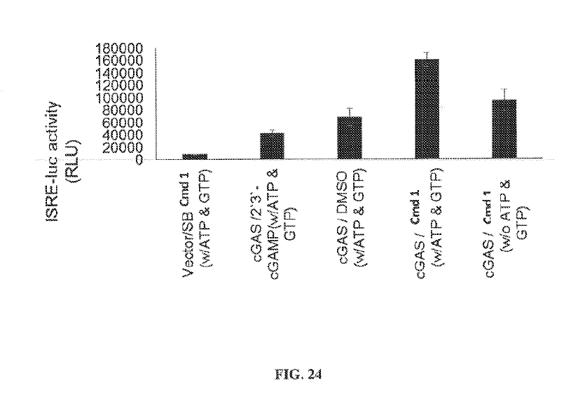

FIG. 23 shows the cells were treated with various concentration of Cmd 7 or DMSO control. After 20 h incubation, RNA was extracted and gene expression was evaluated by Quantitative real time PCR. Fold change was calculated by ΔΔct method.

### EQUIVALENTS

The disclosures of each and every patent, patent application, and publication cited herein are hereby incorporated by reference in their entirety. While this disclosure has been described with reference to specific aspects, it is apparent that other aspects and variations may be devised by others skilled in the art without departing from the true spirit and scope of the disclosure. The appended claims are intended to be construed to include all such aspects and equivalent variations. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

1. A compound of Formula (I): 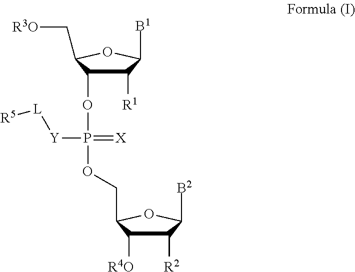
or a pharmaceutically acceptable salt, wherein:
each of B¹ and B² is independently a purinyl nucleobase or pyrimidinyl nucleobase, wherein at least one of B¹ or B² is a purinyl nucleobase;
X is O or S;
Y is O, S, or NR⁶;
L is absent, C₁-C₆ alkyl or C₁-C₆ heteroalkyl, wherein each C₁-C₆ alkyl and C₁-C₆ heteroalkyl is optionally substituted with R⁷;
each of R¹ and R² is independently hydrogen, halo, —CN, C₁-C₂₀ alkyl, or OR⁸, provided that at least one of R¹ and R² is halo, O—C₁-C₂O-alkenyl, or O—C₁-C₂O-alkynyl or R¹ is hydrogen;
each of R³ and R⁴ is independently hydrogen or C₁-C₂₀ alkyl.
R⁵ is hydrogen, C₁-C₂₀ alkyl, C₁-C₂₀ heteroalkyl, cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹;
R⁶ is hydrogen or C₁-C₂₀ alkyl;
R⁷ is halo, —CN, C₁-C₂₀ alkyl, OR⁸, oxo, cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;
R⁸ is hydrogen, C₁-C₂₀ alkynyl, C₁-C₂₀ alkenyl (e.g., C₁-C₆ alkenyl), cycloalkyl, heterocyclyl, aryl, or heteroaryl, wherein each C₁-C₂₀ alkyl, cycloalkyl, heterocyclyl, aryl, or heteroaryl is optionally substituted with 1-5 R¹⁰;
each R⁹ is independently C₁-C₂₀ alkyl, C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R¹⁰; and
each R¹⁰ is independently C₁-C₂₀ alkyl, halo, —CN, OH, O—C₁-C₂₀ alkyl, O—C₁-C₂₀ heteroalkyl, O-aryl, or O-heteroaryl.

2. The compound of claim 1, wherein each of B¹ or B² is independently modified or unmodified adenosinyl, modified or unmodified guanosinyl, modified or unmodified cytosinyl, modified or unmodified thyminyl, or modified or unmodified uracilyl.

3. The compound of claim 2, wherein each of R¹ and R² is independently hydrogen, fluorine, C₁-C₂₀ alkyl, C₁-C₂₀ alkenyl, or O—C₁-C₂₀ alkynyl.

4. The compound of claim 3, wherein each of R¹ and R² is independently fluorine.

5. The compound of claim 1, wherein the compound is a compound of Formula (II):

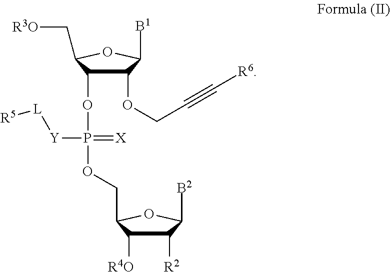

6. The compound of claim 5, wherein R⁶ is hydrogen, C₁-C₂₀ alkyl, cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁹.

7. (canceled)

8. The compound of claim 1, wherein the compound is: 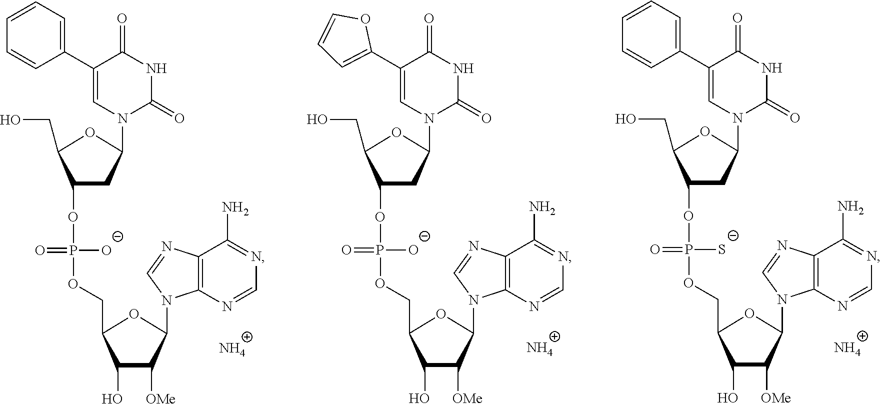 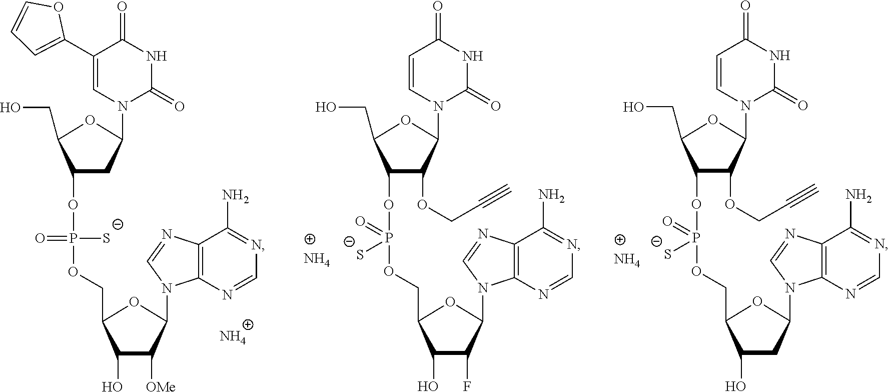 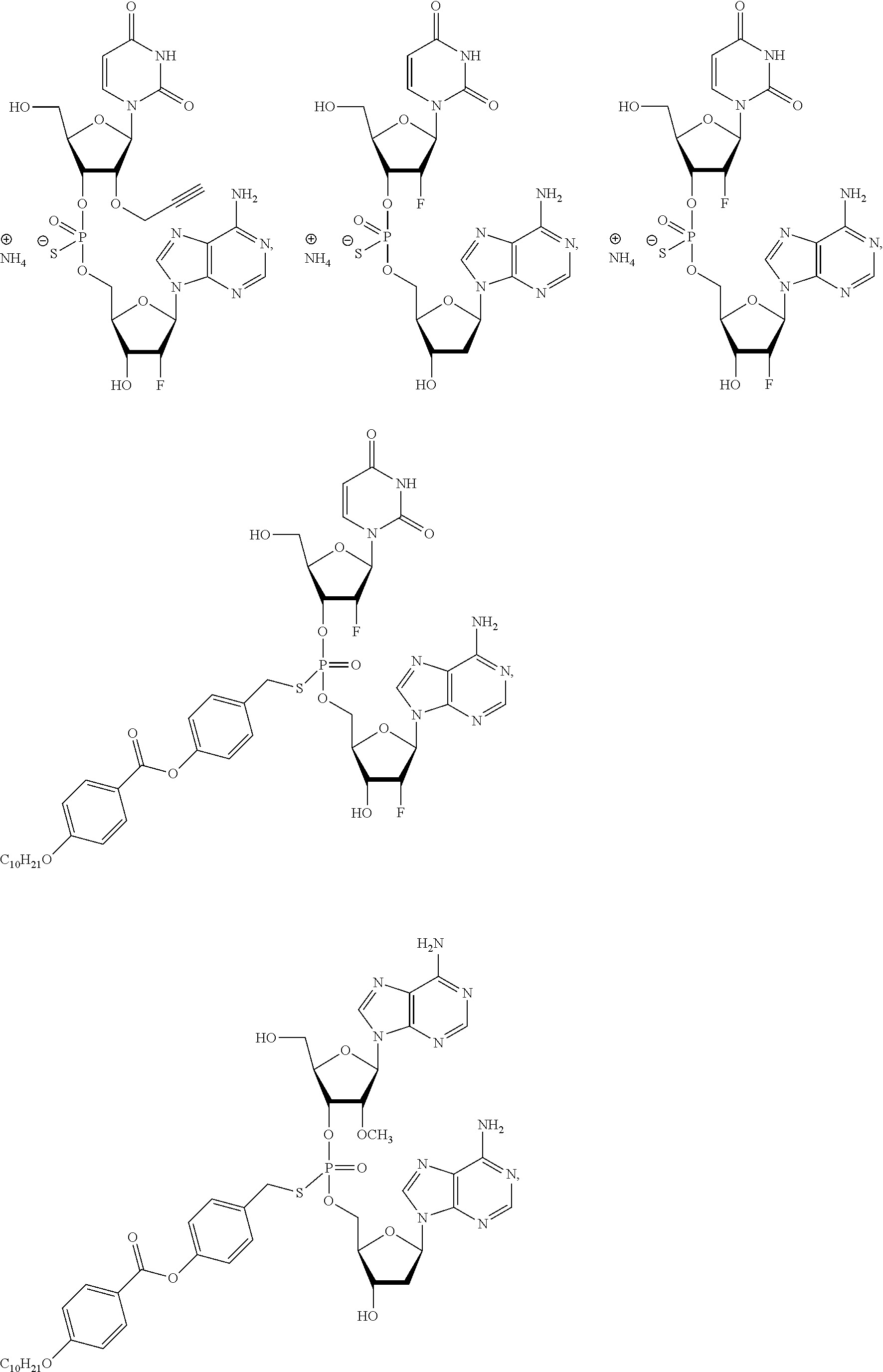 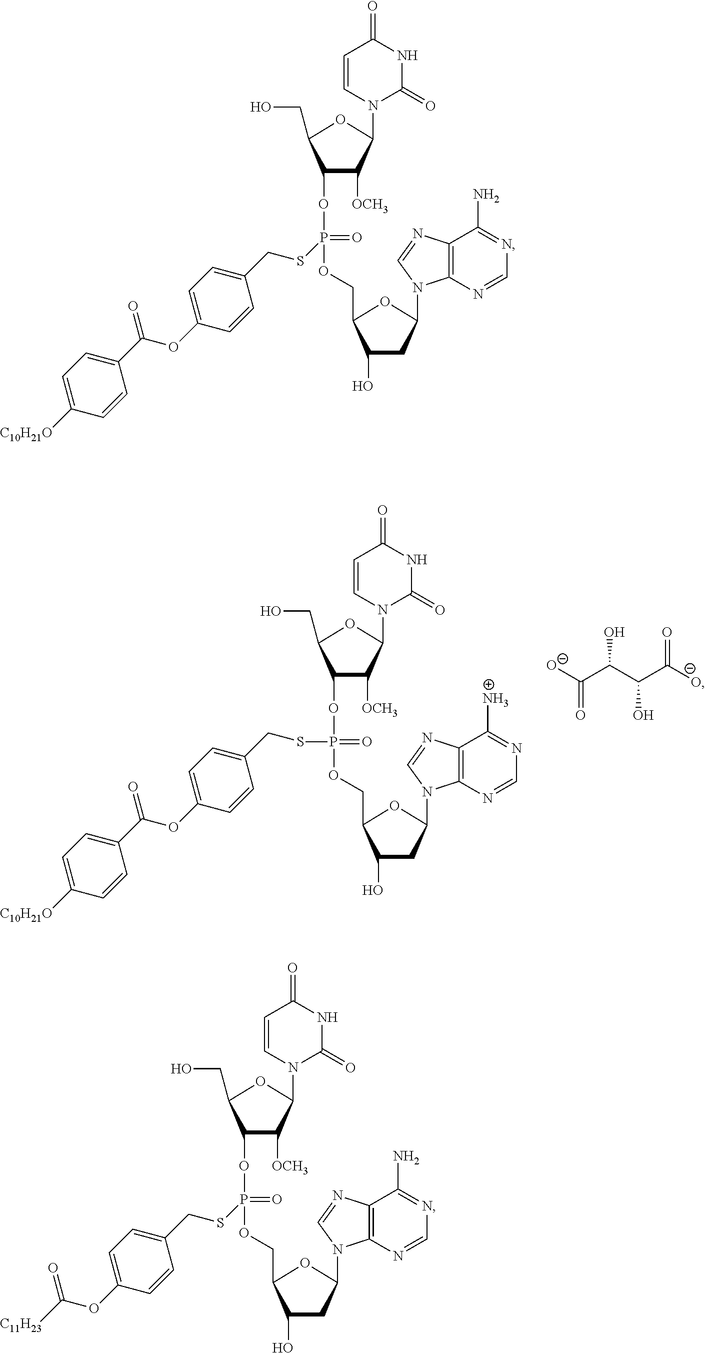 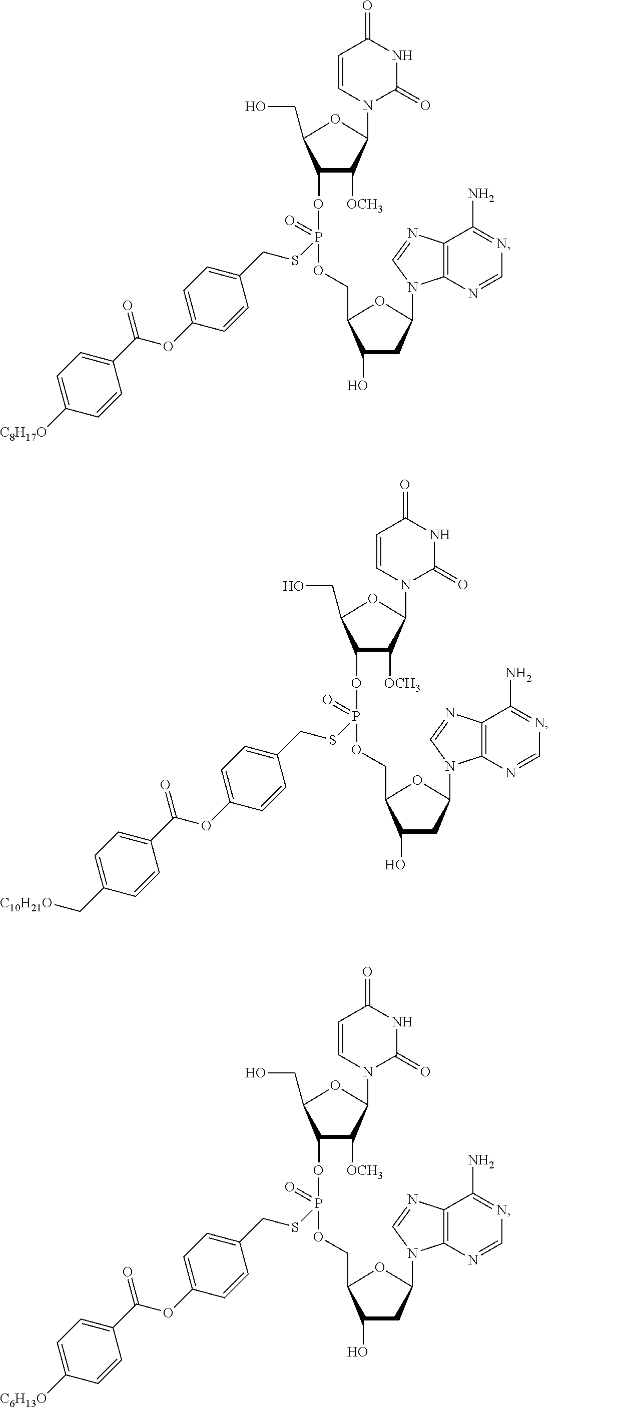 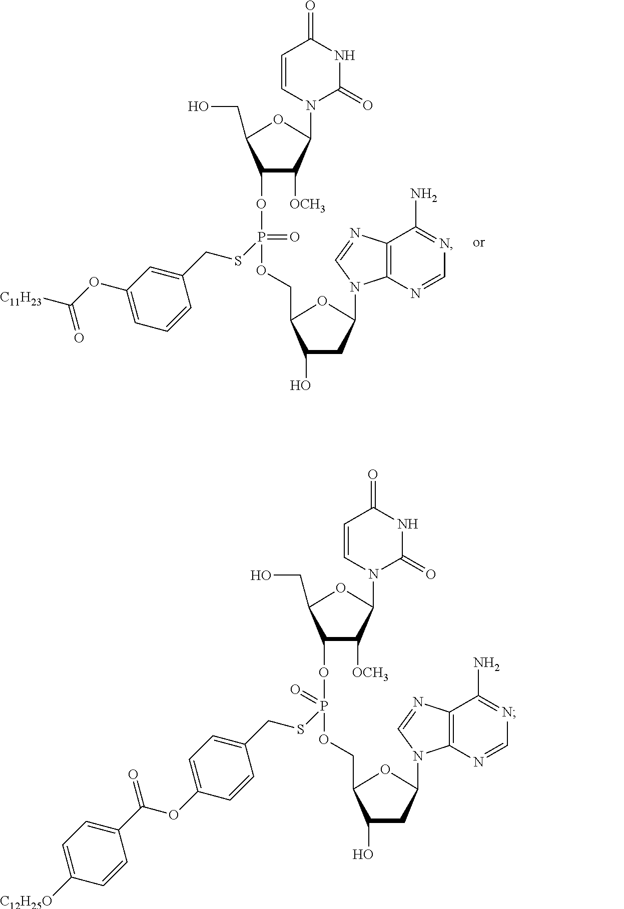
or a pharmaceutically acceptable salt thereof.

9. (canceled)

10. A composition comprising a compound of Formula (III-a) or (III-b): 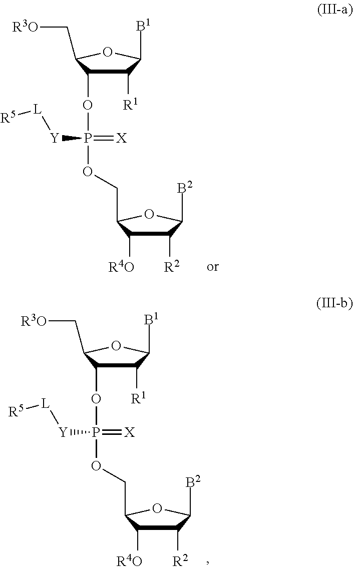
or pharmaceutically acceptable salts thereof, wherein the composition is a mixture of a compound of Formula (III-a) or (III-b).

11. The composition of claim 10, wherein the composition comprises an optically enriched mixture of a compound of Formula (III-a) or (III-b).

12. The composition of claim 10, wherein the composition comprises a compound of Formula (III-a) or (III-b) in an enantiomeric excess of 90%.

13. The compound of claim 1, wherein the compound is of Formula (IV): 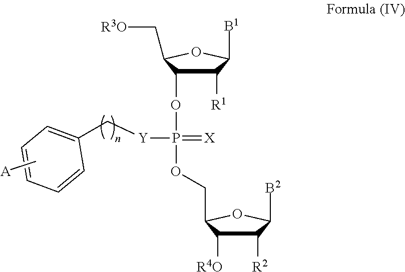
or a pharmaceutically acceptable salt thereof, wherein:
each of B¹ and B² is independently a purinyl nucleobase or pyrimidinyl nucleobase, wherein at least one of B¹ or B² is a purinyl nucleobase;
X is O or S;
Y is O, S, or NR⁵;
n is 1, 2, or 3;
each of R¹ and R² is independently hydrogen, —CN, C₁-C₂₀ alkyl, or OR⁶; each of R³ and R⁴ is independently hydrogen or C₁-C₂₀ alkyl
R⁵ is hydrogen or C₁-C₂₀ alkyl;
R⁶ is hydrogen, C₁-C₂₀ alkyl, C₁-C₂₀ heteroalkyl, cycloalkyl, heterocyclyl, aryl, or heteroaryl, wherein each C₁-C₂₀ alkyl, C₁-C₂₀ heteroalkyl, cycloalkyl, heterocyclyl, aryl, and heteroaryl is optionally substituted with 1-5 R⁷;
each R⁷ is independently C₁-C₂₀ alkyl, C(O)-aryl, C(O)-heteroaryl, OC(O)-aryl, or OC(O)-heteroaryl, wherein each C₁-C₂₀ alkyl, C(O)-aryl, C(O)-heteroaryl, OC(O)-aryl, or OC(O)-heteroaryl is optionally substituted by 1-5 R⁸;
each R⁸ is independently C₁-C₂₀ alkyl, halo, —CN, OH, O—C₁-C₂₀ alkyl, O—C₁-C₂₀ heteroalkyl, O-aryl, or O-heteroaryl; and
A is OC(O)—C₆-C₂₀ alkyl or OC(O)-aryl, wherein aryl is optionally substituted with C₆-C₂₀ alkyl, O—C₆-C₂₀ alkyl or C₁-C₆—O—C₆-C₂₀ alkyl.

14. The compound of claim 13, wherein each of R¹ and R² is independently hydrogen or O—C₁-C₂₀ alkyl.

15. The compound of claim 13, wherein A is OC(O)—C₆-C₂₀ alkyl or OC(O)-aryl, wherein aryl is substituted with C₆-C₂₀ alkyl, O—C₆-C₂₀ alkyl or C₁-C₆—O—C₆-C₂₀ alkyl.

16. The compound of claim 13, wherein each of R³ and R⁴ is independently hydrogen.

17. The compound of claim 15, wherein R¹ is O—C₁-C₂₀ alkyl and R² is hydrogen.

18. (canceled)

19. The composition of claim 10, wherein the composition comprises compounds of Formula (V-a) or (V-b): 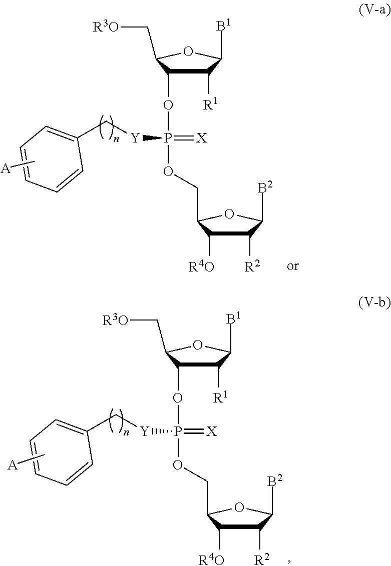
or pharmaceutically acceptable salts thereof, wherein the composition is a mixture of a compound of Formula (V-a) or (V-b).

20. The composition of claim 19, wherein the composition is an optically enriched mixture of a compound of Formula (V-a) or (V-b).

21. The composition of claim 19, wherein the composition comprises a compound of Formula (V-a) or (V-b) in an enantiomeric excess of 90%.

22. The composition of claim 19, wherein the composition comprises: 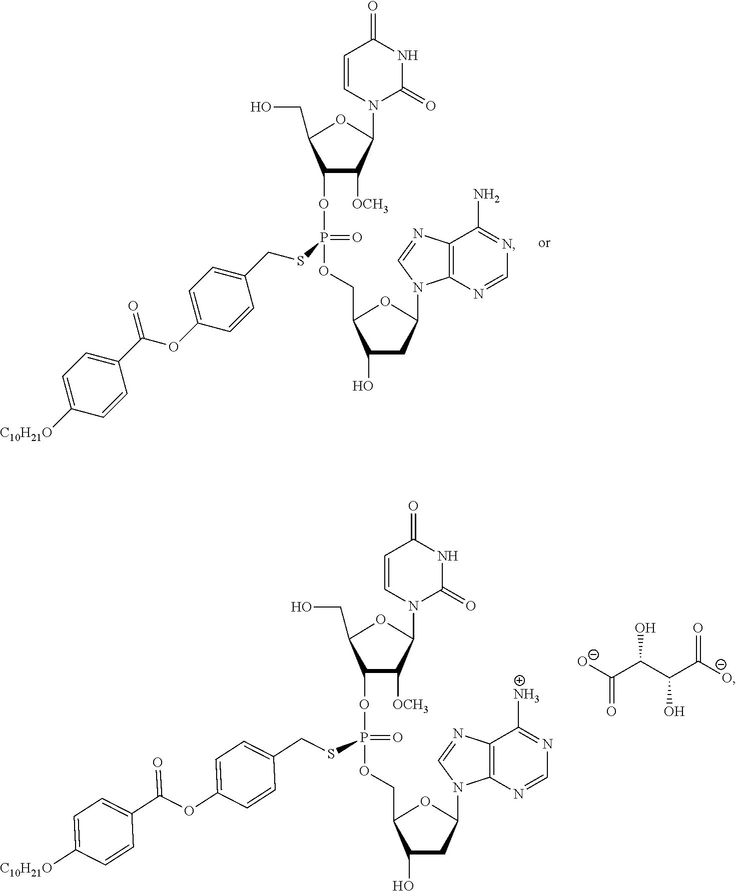
or a pharmaceutically acceptable salt thereof.

23. A method of treating cancer in a subject, the method comprising administering to the subject an effective amount of a compound of claim 1.

24. The method of claim 23, wherein the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

25. The method of claim 24, wherein the cancer is a cancer of the liver.

26. The method of claim 23, further comprising administration of an additional agent.

27. The method of claim 26, wherein the additional agent comprises methotrexate, 5-fluorouracil, doxorubicin, vincristine, bleomycin, vinblastine, dacarbazine, toposide, cisplatin, epirubicin, or sorafenib tosylate.

28. A method of inducing the expression of a pattern recognition receptors (PRRs) for immune-modulation in a subject, the method comprising administering to the subject an effective amount of a compound of claim 1.

29. A method of inducing the expression of a pattern recognition receptors for immunomodulation and inducing a therapeutic response in a subject having cancer, the method comprising administering to the subject an effective amount of a compound of claim 1.

30. A method of treating cancer in a subject, the method comprising administering to the subject an effective amount of a composition of claim 10.

31. The method of claim 30, wherein the cancer is a cancer of the breast, bone, brain, cervix, colon, gastrointestinal tract, eye, gall bladder, lymph nodes, blood, lung, liver, skin, mouth, prostate, ovary, penis, pancreas, uterus, testicles, stomach, thymus, thyroid, or other part of the body.

32. The method of claim 31, wherein the cancer is a cancer of the liver.

33. The method of claim 30, further comprising administration of an additional agent.

34. The method of claim 33, wherein the additional agent comprises methotrexate, 5-fluorouracil, doxorubicin, vincristine, bleomycin, vinblastine, dacarbazine, toposide, cisplatin, epirubicin, or sorafenib tosylate.

35. A method of inducing the expression of a pattern recognition receptors (PRRs) for immune-modulation in a subject, the method comprising administering to the subject an effective amount of a composition of claim 10.

36. A method of inducing the expression of a pattern recognition receptors for immunomodulation and inducing a therapeutic response in a subject having cancer, the method comprising administering to the subject an effective amount of a composition of claim 10.
